SEQUENCE LISTING (21 sequences)

SEQ ID NO: 1 - protein, 332 aa, Homo Sapiens
MVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIIITLLNSTDTDAQSFTVNIDNVIDSVICSSLLASICSLLSIAVDRYFTIFYALQYHNIMTVKRVGIIISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRY

SEQ ID NO: 2 - protein, 30 aa, Homo Sapiens
YEQLFVSPEVFVTLGVISLLENILVIVAIA

SEQ ID NO: 3 - protein, 30 aa, Homo Sapiens
PMYFFICSLAVADMLVSVSNGSETIIITLL

SEQ ID NO: 4 - protein, 31 aa, Homo Sapiens
NIDNVIDSVICSSLLASICSLLSIAVDRYFT

SEQ ID NO: 5 - protein, 24 aa, Homo Sapiens
VKRVGIIISCIWAACTVSGILFII

SEQ ID NO: 6 - protein, 29 aa, Homo Sapiens
DSSAVIICLITMFFTMLALMASLYVHMFL

SEQ ID NO: 7 - protein, 32 aa, Homo Sapiens
ANMKGAITLTILIGVFVVCWAPFFLHLIFYIS

SEQ ID NO: 8 - protein, 24 aa, Homo Sapiens
SHFNLYLILIMCNSIIDPLIYALR

SEQ ID NO: 9 - protein, 12 aa, Homo Sapiens
NSTDTDAQSFTV

SEQ ID NO: 10 - protein, 2 aa, Homo Sapiens
YS

SEQ ID NO: 11 - protein, 11 aa, Homo Sapiens
CPQNPYCVCFM

SEQ ID NO: 12 - protein, 15 aa, Homo Sapiens
YEQLFVSPEVFVTLG

SEQ ID NO: 13 - protein, 42 aa, Homo Sapiens
VSVSNGSETIIITLLNSTDTDAQSFTVNIDNVIDSVICSSLL

SEQ ID NO: 14 - protein, 32 aa, Homo Sapiens
CIWAACTVSGILFIIYSDSSAVIICLITMFFT

SEQ ID NO: 15 - protein, 41 aa, Homo Sapiens
VCWAPFFLHLIFYISCPQNPYCVCFMSHFNLYLILIMCNSI

SEQ ID NO: 16 - protein, 15 aa, Homo Sapiens
VSVSNGSETIIITLL

SEQ ID NO: 17 - protein, 15 aa, Homo Sapiens
NIDNVIDSVICSSLL

SEQ ID NO: 18 - protein, 15 aa, Homo Sapiens
CIWAACTVSGILFII

SEQ ID NO: 19 - protein, 15 aa, Homo Sapiens
DSSAVIICLITMFFT

SEQ ID NO: 20 - protein, 15 aa, Homo Sapiens
VCWAPFFLHLIFYIS

SEQ ID NO: 21 - protein, 15 aa, Homo Sapiens
SHFNLYLILIMCNSI

(19) United States
(12) Patent Application Publication
(10) Pub. No.: US 2005/0171000 A1
(43) Pub. Date: Aug. 4, 2005

(54) Three dimensional coordinates of melanocortin-4 receptors

(75) Inventors: Anny-Odile Colson, Loveland, OH (US); Glen Edward Mieling, West Chester, OH (US); Katherine King Mieling, West Chester, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/414,521

(22) Filed: Apr. 15, 2003

(51) Int. Cl. A61K 38/00; G06F 19/00; G01N 33/48; G01N 33/50

(52) U.S. Cl. 514/2; 702/19

(57) ABSTRACT

Three-dimensional coordinates of melanocortin-4 receptors are useful in computer aided drug design.

### FIELD OF THE INVENTION

The present invention relates to three-dimensional structures of G protein-coupled receptors, especially melanocortin-4 receptors, and structures derived therefrom.

### BACKGROUND OF THE INVENTION

I. GPCRs

Most eukaryotic organisms utilize guanine nucleotide-binding regulatory protein (hereinafter, referred to as “G protein”)-coupled receptors (G protein-coupled receptors; hereinafter, frequently referred to as “GPCRs”) to respond to a variety of stimuli such as Ca²⁺, amines, hormones, peptides, and even large proteins. Each GPCR is composed of an extracellular region, an intracellular region and a transmembrane bundle of seven α-helices. The binding of specific ligands to the extracellular or transmembrane domain evokes conformational changes that are transmitted to the intracellular region. The conformational switch from inactive to active state in the intracellular region produces a catalytically competent receptor that is responsible for binding and nucleotide exchange (GDP⇄GTP) on the α-subunit of several hundreds of G proteins of a specific subtype. The α-and/or A′-subunit of the G proteins activates the next target protein(s) to evoke further intracellular responses. Because all of several hundreds of members in the GPCR family appear to share a common motif in their transmembrane structure and a common topology, it is generally accepted that a similar switching mechanism functions in all of these receptors.

II. MC4-R

Melanocortin peptides or melanotropins, α-MSH (melanocortin stimulating hormone), β-MSH, γ-MSH and ACTH (adrenocorticotropic hormone), are involved in many physiological functions in vertebrates, mammals and in humans. They regulate skin pigmentation and steroid production, modulate immune responses and learning processes, influence energy balance, growth and regeneration of nerves, and several other functions as well.

Five human receptors are known which interact with melanotropins, hMC1-R to hMC5-R. The receptors are seven-helix transmembrane-spanning receptors and belong to the superfamily of GPCRs; their activation leads to elevation of cAMP. The melanocortin receptors 1, 3, 4 and 5 recognize α-MSH, β-MSH, γ-MSH, while melanocortin receptor 2 recognizes only ACTH (Hruby, V. J. et al. J. Med. Chem. 1995, 38(18), 3454).

Considerable attention has recently focused on melanocortin receptors 3 and 4 that are widely expressed in the central nervous system, and also on melanocortin receptor 5, found in the brain and in various peripheral tissues. The physiological role of hMC3-R and hMC5-R is not well defined, although hMC5-R has recently been implicated in control of lipid and pheromone production in exocrine glands (Caldwell, H. K. et al. Chemical Senses, 2002, 27(1), 91). Rapidly growing pharmacological and genetic evidence suggests that hMC4-R is involved in regulation of the energy balance and body weight in rodents. The role of MC4-R in regulation of food intake and body weight is supported by results obtained from agonist/antagonist administration in rats and from murine genetics (Fan, W. et al. Nature, 1997, 385, 165; Huszer, D. et al. Cell, 1997, 88, 131; Klebig, M. L. et al. Proc. Nat]. Acad. Sci. 1995, 92, 4728; Karbon, W. et al. Abstract from the Nineteenth Annual Winter Neuropeptide Conference, 1998; Seely, R. J. et al. Nature, 1997, 390, 349; Comuzzie, A. G. Nat. Gen. 1997, 15, 273; Chagnon, Y. C. et al. Mol. Med. 1997, 3, 663; Lee, F. et al. 1997, World Patent Publication WO 97/47316; Shutter, J. R. et al. Gen. & Dev. 1997, 11, 593). Intraventricular administration of the agonist MTII reduced food intake and conversely, the antagonist SHU9119 increased food intake and body weight. Mice genetically deficient in the melanocortin receptor 4 develop obesity. It could be anticipated therefore that compounds inhibiting MC4-R might be useful in the treatment of weight control. As such, agonists of MC4-R may be useful as therapeutic agents for the diagnosis, treatment or prevention of diseases and disorders mediated through the MC4 receptor, more particularly, disorders related to obesity of any cause in humans. Thus there is a need to provide for potent and selective agonists of the MC4 receptor.

III. Three-Dimensional Structural Information

The biological properties of a protein depend directly on the protein's three-dimensional (3D) conformation. In turn, the 3D conformation determines the activity of enzymes, the capacity and specificity of binding proteins, and the potential for small molecules to interact with the receptor. Models at the atomic level are needed to obtain insight into the mechanisms of MC4 receptor activation, and the source of interactions between specific ligands and MC4-R. Thus, there is a need for 3D structure models for the MC4 receptor.

### SUMMARY OF THE INVENTION

The present invention attempts to address this need by providing a 3D structure of human MC4 receptor that is useful in identifying potent and selective agonists or antagonist of the MC4 receptor for the treatment of MC4-R mediated disorders.

One aspect of the invention provides for a method of identifying a drug candidate useful for the treatment of a melanocortin receptor-4 (MC4-R) mediated disorder comprising the steps of:
(a) using a three-dimensional (3D) structure of MC4-R according to Table 1;
(b) employing said 3D structure to design or select in silico said drug candidate.

Another aspect of the invention provides a G-protein-coupled receptor selected from the group consisting of the following (a) or (b):
(a) a G protein-coupled receptor having three-dimensional structure I defined by the atomic coordinates as shown in Table 1;
(b) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.

These and other objects, features, and advantages will become apparent to those of ordinary skill in the art from a reading of the following detailed description and the appended claims.

### SEQUENCE LISTING DESCRIPTION

Each of the protein sequences in the sequence listing is shown in Table A.

TABLE A

| | | |
|---|---|---|
| Sequence | | Position on |
| Description | SEQ ID NO: | SEQ ID NO: 1 |

| | | |
|---|---|---|
| MC4R¹ | 1 | — |
| HI | 2 | 41-70 |
| HII | 3 | 78-107 |
| HIII | 4 | 120-150 |
| HIV | 5 | 163-186 |
| HV | 6 | 189-217 |
| HVI | 7 | 239-270 |
| HVII | 8 | 282-305 |
| ECI | 9 | 108-119 |
| ECII | 10 | 187-188 |
| ECIII | 11 | 271-281 |
| THI | 12 | 41-55 |
| THII-ECI-THIII | 13 | 93-134 |
| THIV-ECII-THV | 14 | 172-203 |
| THVI-ECIII- | 15 | 256-296 |
| THVII | | |
| THII | 16 | 93-107 |
| THIII | 17 | 120-134 |
| THIV | 18 | 172-186 |
| THV | 19 | 189-203 |
| THVI | 20 | 256-270 |
| THVII | 21 | 282-296 |

¹The corresponding human MC4R accession number is P32245.

### DETAILED DESCRIPTION OF THE INVENTION

I. 3D Coordinates of MC4-R

G protein-coupled receptors (GPCRs) function in a number of signaling pathways, converting a variety of external stimuli to activate multiple copies of G protein subtypes specific to the relevant G protein-coupled receptor. GPCRs, including MC4-R, share many structural features, including a bundle of seven transmembrane α-helices connected by six loops of varying lengths. In the present invention, in silico 3D coordinates of the transmembrane and extra-cellular regions of MC4-R are provided. These coordinates were obtained by homology from a model constructed for MC1-R utilizing experimental mutagenesis data, the 3D structure of bacterial rhodopsin and in-house SAR data. The extra-cellular loops were subsequently modeled employing molecular dynamics simulations (Colson, A. O.; Perlman, J. H.; Smolyar, A.; Gershengorn, M. C.; Osman, R. O. Biophys. J. 1998, 74, 1087). The helices in the model were later refined using the 3D coordinates of mammalian rhodopsin (Palczewski, K.; Kumasaka, T.; Hori, T.; Behnke, C. A.; Motoshima, H.; Fox, B. A.; Trong, I. L.; Teller, D. C.; Okada, T.; Stenkamp, R. E.; Yamamoto, M.; Miyano, M. Science, 2000, 289, 739). Further refinement, such as the removal of kinks from helices II, IV and V, and coordinates modification in view of SAR data generated in-house, was also performed.

As a result of the above analysis, the 3D structure of human MC4-R can be obtained as 3D coordinates. The atomic coordinates obtained by the invention are shown in Table 1 below.

TABLE 1

| | | | | | | | |
|---|---|---|---|---|---|---|---|
| ATOM | 1 | N | TYR | 41 | −42.17 | 22.835 | 20.182 |
| ATOM | 2 | CA | TYR | 41 | −43.253 | 21.888 | 20.437 |
| ATOM | 3 | C | TYR | 41 | −43.856 | 21.393 | 19.14 |
| ATOM | 4 | O | TYR | 41 | −43.754 | 20.208 | 18.867 |
| ATOM | 5 | CB | TYR | 41 | −44.313 | 22.54 | 21.358 |
| ATOM | 6 | CG | TYR | 41 | −43.737 | 22.689 | 22.774 |
| ATOM | 7 | CD1 | TYR | 41 | −43.202 | 23.907 | 23.205 |
| ATOM | 8 | CD2 | TYR | 41 | −43.739 | 21.595 | 23.648 |
| ATOM | 9 | CE1 | TYR | 41 | −42.691 | 24.038 | 24.501 |
| ATOM | 10 | CE2 | TYR | 41 | −43.23 | 21.726 | 24.943 |
| ATOM | 11 | CZ | TYR | 41 | −42.706 | 22.946 | 25.378 |
| ATOM | 12 | OH | TYR | 41 | −42.207 | 23.048 | 26.68 |
| ATOM | 13 | HB1 | TYR | 41 | −44.611 | 23.522 | 20.96 |
| ATOM | 14 | HB2 | TYR | 41 | −45.214 | 21.912 | 21.399 |
| ATOM | 15 | HD1 | TYR | 41 | −43.178 | 24.76 | 22.534 |
| ATOM | 16 | HD2 | TYR | 41 | −44.134 | 20.638 | 23.325 |
| ATOM | 17 | HE1 | TYR | 41 | −42.285 | 24.993 | 24.816 |
| ATOM | 18 | HE2 | TYR | 41 | −43.24 | 20.876 | 25.618 |
| ATOM | 19 | HH | TYR | 41 | −41.876 | 23.91 | 26.906 |
| ATOM | 20 | H | TYR | 41 | −42.203 | 23.429 | 19.373 |
| ATOM | 21 | H1 | TYR | 41 | −41.443 | 22.948 | 20.861 |
| ATOM | 22 | HA | TYR | 41 | −42.843 | 21.005 | 20.958 |
| ATOM | 23 | N | GLU | 42 | −44.472 | 22.257 | 18.305 |
| ATOM | 24 | CA | GLU | 42 | −44.935 | 21.79 | 16.998 |
| ATOM | 25 | C | GLU | 42 | −43.783 | 21.145 | 16.259 |
| ATOM | 26 | O | GLU | 42 | −43.969 | 20.094 | 15.666 |
| ATOM | 27 | CB | GLU | 42 | −45.602 | 22.894 | 16.124 |
| ATOM | 28 | CG | GLU | 42 | −44.637 | 23.977 | 15.562 |
| ATOM | 29 | CD | GLU | 42 | −43.955 | 24.75 | 16.659 |
| ATOM | 30 | OE1 | GLU | 42 | −44.365 | 25.911 | 16.926 |
| ATOM | 31 | OE2 | GLU | 42 | −43.007 | 24.192 | 17.276 |
| ATOM | 32 | HB2 | GLU | 42 | −46.086 | 22.403 | 15.262 |
| ATOM | 33 | HB1 | GLU | 42 | −46.389 | 23.387 | 16.718 |
| ATOM | 34 | HG2 | GLU | 42 | −45.216 | 24.683 | 14.945 |
| ATOM | 35 | HG1 | GLU | 42 | −43.877 | 23.523 | 14.908 |
| ATOM | 36 | H | GLU | 42 | −44.567 | 23.225 | 18.539 |
| ATOM | 37 | HA | GLU | 42 | −45.699 | 21.017 | 17.187 |
| ATOM | 38 | N | GLN | 43 | −42.572 | 21.748 | 16.3 |
| ATOM | 39 | CA | GLN | 43 | −41.429 | 21.147 | 15.618 |
| ATOM | 40 | C | GLN | 43 | −41.245 | 19.723 | 16.093 |
| ATOM | 41 | O | GLN | 43 | −40.87 | 18.877 | 15.297 |
| ATOM | 42 | CB | GLN | 43 | −40.102 | 21.913 | 15.874 |
| ATOM | 43 | CG | GLN | 43 | −40.085 | 23.327 | 15.231 |
| ATOM | 44 | CD | GLN | 43 | −38.717 | 23.949 | 15.392 |
| ATOM | 45 | OE1 | GLN | 43 | −38.551 | 24.823 | 16.227 |
| ATOM | 46 | NE2 | GLN | 43 | −37.703 | 23.519 | 14.612 |
| ATOM | 47 | H | GLN | 43 | −42.444 | 22.597 | 16.819 |
| ATOM | 48 | HB2 | GLN | 43 | −39.274 | 21.323 | 15.446 |
| ATOM | 49 | HB1 | GLN | 43 | −39.931 | 22.001 | 16.96 |
| ATOM | 50 | HG2 | GLN | 43 | −40.838 | 23.981 | 15.697 |
| ATOM | 51 | HG1 | GLN | 43 | −40.318 | 23.26 | 14.157 |
| ATOM | 52 | HE21 | GLN | 43 | −37.835 | 22.806 | 13.924 |
| ATOM | 53 | HE22 | GLN | 43 | −36.797 | 23.929 | 14.726 |
| ATOM | 54 | HA | GLN | 43 | −41.633 | 21.134 | 14.535 |
| ATOM | 55 | N | LEU | 44 | −41.502 | 19.435 | 17.388 |
| ATOM | 56 | CA | LEU | 44 | −41.408 | 18.058 | 17.854 |
| ATOM | 57 | C | LEU | 44 | −42.62 | 17.319 | 17.333 |
| ATOM | 58 | O | LEU | 44 | −42.44 | 16.318 | 16.661 |
| ATOM | 59 | CB | LEU | 44 | −41.344 | 17.92 | 19.404 |
| ATOM | 60 | CG | LEU | 44 | −39.97 | 18.31 | 20.029 |
| ATOM | 61 | CD1 | LEU | 44 | −39.547 | 19.769 | 19.703 |
| ATOM | 62 | CD2 | LEU | 44 | −40.023 | 18.112 | 21.571 |
| ATOM | 63 | HB2 | LEU | 44 | −42.144 | 18.513 | 19.872 |
| ATOM | 64 | HB1 | LEU | 44 | −41.533 | 16.862 | 19.653 |
| ATOM | 65 | HG | LEU | 44 | −39.195 | 17.633 | 19.628 |
| ATOM | 66 | HD13 | LEU | 44 | −39.362 | 19.895 | 18.626 |
| ATOM | 67 | HD12 | LEU | 44 | −38.614 | 20.023 | 20.229 |
| ATOM | 68 | HD11 | LEU | 44 | −40.327 | 20.478 | 20.021 |
| ATOM | 69 | HD23 | LEU | 44 | −40.771 | 18.786 | 22.019 |
| ATOM | 70 | HD22 | LEU | 44 | −39.043 | 18.329 | 22.024 |
| ATOM | 71 | HD21 | LEU | 44 | −40.293 | 17.074 | 21.821 |
| ATOM | 72 | H | LEU | 44 | −41.827 | 20.135 | 18.024 |
| ATOM | 73 | HA | LEU | 44 | −40.501 | 17.582 | 17.443 |
| ATOM | 74 | N | PHE | 45 | −43.86 | 17.772 | 17.63 |
| ATOM | 75 | CA | PHE | 45 | −45.039 | 16.97 | 17.296 |
| ATOM | 76 | C | PHE | 45 | −45.242 | 16.72 | 15.818 |
| ATOM | 77 | O | PHE | 45 | −45.949 | 15.772 | 15.509 |
| ATOM | 78 | CB | PHE | 45 | −46.344 | 17.575 | 17.882 |
| ATOM | 79 | CG | PHE | 45 | −46.298 | 17.607 | 19.419 |
| ATOM | 80 | CD1 | PHE | 45 | −46.255 | 18.819 | 20.119 |
| ATOM | 81 | CD2 | PHE | 45 | −46.307 | 16.405 | 20.137 |
| ATOM | 82 | CE1 | PHE | 45 | −46.195 | 18.824 | 21.516 |
| ATOM | 83 | CE2 | PHE | 45 | −46.198 | 16.408 | 21.531 |
| ATOM | 84 | CZ | PHE | 45 | −46.142 | 17.62 | 22.223 |
| ATOM | 85 | HB2 | PHE | 45 | −46.491 | 18.581 | 17.46 |
| ATOM | 86 | HB1 | PHE | 45 | −47.211 | 16.96 | 17.591 |
| ATOM | 87 | HD1 | PHE | 45 | −46.273 | 19.763 | 19.585 |
| ATOM | 88 | HD2 | PHE | 45 | −46.402 | 15.46 | 19.614 |
| ATOM | 89 | HE1 | PHE | 45 | −46.194 | 19.762 | 22.058 |
| ATOM | 90 | HE2 | PHE | 45 | −46.162 | 15.472 | 22.08 |
| ATOM | 91 | HZ | PHE | 45 | −46.06 | 17.625 | 23.305 |
| ATOM | 92 | H | PHE | 45 | −43.983 | 18.635 | 18.124 |
| ATOM | 93 | HA | PHE | 45 | −44.89 | 15.978 | 17.751 |
| ATOM | 94 | N | VAL | 46 | −44.667 | 17.501 | 14.876 |
| ATOM | 95 | CA | VAL | 46 | −44.821 | 17.135 | 13.465 |
| ATOM | 96 | C | VAL | 46 | −44.033 | 15.881 | 13.143 |
| ATOM | 97 | O | VAL | 46 | −44.515 | 15.085 | 12.353 |
| ATOM | 98 | CB | VAL | 46 | −44.469 | 18.245 | 12.427 |
| ATOM | 99 | CG1 | VAL | 46 | −45.406 | 19.476 | 12.578 |
| ATOM | 100 | CG2 | VAL | 46 | −42.974 | 18.672 | 12.461 |
| ATOM | 101 | HA | VAL | 46 | −45.885 | 16.895 | 13.294 |
| ATOM | 102 | HB | VAL | 46 | −44.655 | 17.825 | 11.421 |
| ATOM | 103 | HG23 | VAL | 46 | −42.711 | 19.115 | 13.429 |
| ATOM | 104 | HG22 | VAL | 46 | −42.778 | 19.423 | 11.68 |
| ATOM | 105 | HG21 | VAL | 46 | −42.311 | 17.814 | 12.275 |
| ATOM | 106 | HG13 | VAL | 46 | −46.459 | 19.175 | 12.47 |
| ATOM | 107 | HG12 | VAL | 46 | −45.184 | 20.225 | 11.802 |
| ATOM | 108 | HG11 | VAL | 46 | −45.282 | 19.95 | 13.56 |
| ATOM | 109 | H | VAL | 46 | −44.118 | 18.299 | 15.134 |
| ATOM | 110 | N | SER | 47 | −42.819 | 15.654 | 13.697 |
| ATOM | 111 | CA | SER | 47 | −42.042 | 14.502 | 13.235 |
| ATOM | 112 | C | SER | 47 | −42.762 | 13.186 | 13.482 |
| ATOM | 113 | O | SER | 47 | −42.826 | 12.411 | 12.54 |
| ATOM | 114 | CB | SER | 47 | −40.56 | 14.53 | 13.691 |
| ATOM | 115 | OG | SER | 47 | −39.977 | 15.765 | 13.236 |
| ATOM | 116 | HB2 | SER | 47 | −40.467 | 14.466 | 14.783 |
| ATOM | 117 | HB1 | SER | 47 | −40.029 | 13.674 | 13.242 |
| ATOM | 118 | HG | SER | 47 | −39.063 | 15.866 | 13.485 |
| ATOM | 119 | H | SER | 47 | −42.42 | 16.274 | 14.377 |
| ATOM | 120 | HA | SER | 47 | −41.97 | 14.618 | 12.139 |
| ATOM | 121 | N | PRO | 48 | −43.35 | 12.858 | 14.662 |
| ATOM | 122 | CA | PRO | 48 | −44.222 | 11.697 | 14.744 |
| ATOM | 123 | C | PRO | 48 | −45.25 | 11.674 | 13.641 |
| ATOM | 124 | O | PRO | 48 | −45.415 | 10.631 | 13.032 |
| ATOM | 125 | CB | PRO | 48 | −44.891 | 11.917 | 16.125 |
| ATOM | 126 | CG | PRO | 48 | −43.83 | 12.71 | 16.92 |
| ATOM | 127 | CD | PRO | 48 | −43.219 | 13.657 | 15.864 |
| ATOM | 128 | HD2 | PRO | 48 | −43.838 | 14.551 | 15.761 |
| ATOM | 129 | HB2 | PRO | 48 | −45.786 | 12.554 | 16.032 |
| ATOM | 130 | HB1 | PRO | 48 | −45.186 | 10.97 | 16.603 |
| ATOM | 131 | HG1 | PRO | 48 | −44.244 | 13.25 | 17.787 |
| ATOM | 132 | HG2 | PRO | 48 | −43.053 | 12.016 | 17.274 |
| ATOM | 133 | HD1 | PRO | 48 | −42.189 | 13.913 | 16.139 |
| ATOM | 134 | H5 | PRO | 48 | −43.616 | 10.78 | 14.751 |
| ATOM | 135 | N | GLU | 49 | −45.951 | 12.796 | 13.359 |
| ATOM | 136 | CA | GLU | 49 | −46.976 | 12.762 | 12.316 |
| ATOM | 137 | C | GLU | 49 | −46.335 | 12.373 | 11.005 |
| ATOM | 138 | O | GLU | 49 | −46.871 | 11.531 | 10.301 |
| ATOM | 139 | CB | GLU | 49 | −47.705 | 14.126 | 12.166 |
| ATOM | 140 | CG | GLU | 49 | −48.833 | 14.069 | 11.103 |
| ATOM | 141 | CD | GLU | 49 | −49.525 | 15.405 | 11.044 |
| ATOM | 142 | OE1 | GLU | 49 | −48.941 | 16.346 | 10.442 |
| ATOM | 143 | OE2 | GLU | 49 | −50.649 | 15.524 | 11.601 |
| ATOM | 144 | HB2 | GLU | 49 | −48.142 | 14.406 | 13.139 |
| ATOM | 145 | HB1 | GLU | 49 | −46.987 | 14.907 | 11.874 |
| ATOM | 146 | HG2 | GLU | 49 | −49.562 | 13.284 | 11.362 |
| ATOM | 147 | HG1 | GLU | 49 | −48.421 | 13.843 | 10.107 |
| ATOM | 148 | H | GLU | 49 | −45.77 | 13.66 | 13.836 |
| ATOM | 149 | HA | GLU | 49 | −47.723 | 12.001 | 12.595 |
| ATOM | 150 | N | VAL | 50 | −45.171 | 12.965 | 10.663 |
| ATOM | 151 | CA | VAL | 50 | −44.507 | 12.568 | 9.425 |
| ATOM | 152 | C | VAL | 50 | −44.222 | 11.086 | 9.502 |
| ATOM | 153 | O | VAL | 50 | −44.503 | 10.379 | 8.547 |
| ATOM | 154 | CB | VAL | 50 | −43.196 | 13.363 | 9.148 |
| ATOM | 155 | CG1 | VAL | 50 | −42.425 | 12.757 | 7.941 |
| ATOM | 156 | CG2 | VAL | 50 | −43.501 | 14.864 | 8.876 |
| ATOM | 157 | HB | VAL | 50 | −42.542 | 13.292 | 10.033 |
| ATOM | 158 | HG23 | VAL | 50 | −44.115 | 14.969 | 7.968 |
| ATOM | 159 | HG22 | VAL | 50 | −42.564 | 15.424 | 8.73 |
| ATOM | 160 | HG21 | VAL | 50 | −44.042 | 15.327 | 9.714 |
| ATOM | 161 | HG13 | VAL | 50 | −42.042 | 11.755 | 8.187 |
| ATOM | 162 | HG12 | VAL | 50 | −41.567 | 13.39 | 7.676 |
| ATOM | 163 | HG11 | VAL | 50 | −43.083 | 12.681 | 7.062 |
| ATOM | 164 | H | VAL | 50 | −44.738 | 13.638 | 11.267 |
| ATOM | 165 | HA | VAL | 50 | −45.201 | 12.743 | 8.586 |
| ATOM | 166 | N | PHE | 51 | −43.667 | 10.577 | 10.624 |
| ATOM | 167 | CA | PHE | 51 | −43.405 | 9.142 | 10.698 |
| ATOM | 168 | C | PHE | 51 | −44.673 | 8.324 | 10.713 |
| ATOM | 169 | O | PHE | 51 | −44.615 | 7.19 | 10.266 |
| ATOM | 170 | CB | PHE | 51 | −42.547 | 8.75 | 11.925 |
| ATOM | 171 | CG | PHE | 51 | −41.169 | 9.426 | 11.884 |
| ATOM | 172 | CD1 | PHE | 51 | −40.324 | 9.241 | 10.783 |
| ATOM | 173 | CD2 | PHE | 51 | −40.731 | 10.228 | 12.943 |
| ATOM | 174 | CE1 | PHE | 51 | −39.113 | 9.929 | 10.7 |
| ATOM | 175 | CE2 | PHE | 51 | −39.457 | 10.801 | 12.926 |
| ATOM | 176 | CZ | PHE | 51 | −38.645 | 10.653 | 11.8 |
| ATOM | 177 | HB2 | PHE | 51 | −43.108 | 9.027 | 12.829 |
| ATOM | 178 | HB1 | PHE | 51 | −42.396 | 7.659 | 11.936 |
| ATOM | 179 | HD1 | PHE | 51 | −40.591 | 8.558 | 9.985 |
| ATOM | 180 | HD2 | PHE | 51 | −41.378 | 10.409 | 13.79 |
| ATOM | 181 | HE1 | PHE | 51 | −38.539 | 9.888 | 9.782 |
| ATOM | 182 | HE2 | PHE | 51 | −39.099 | 11.359 | 13.786 |
| ATOM | 183 | HZ | PHE | 51 | −37.654 | 11.093 | 11.783 |
| ATOM | 184 | H | PHE | 51 | −43.449 | 11.159 | 11.411 |
| ATOM | 185 | HA | PHE | 51 | −42.871 | 8.853 | 9.782 |
| ATOM | 186 | N | VAL | 52 | −45.827 | 8.832 | 11.193 |
| ATOM | 187 | CA | VAL | 52 | −47.061 | 8.071 | 11.016 |
| ATOM | 188 | C | VAL | 52 | −47.274 | 7.98 | 9.523 |
| ATOM | 189 | O | VAL | 52 | −47.498 | 6.89 | 9.025 |
| ATOM | 190 | CB | VAL | 52 | −48.298 | 8.695 | 11.73 |
| ATOM | 191 | CG1 | VAL | 52 | −49.616 | 7.991 | 11.297 |
| ATOM | 192 | CG2 | VAL | 52 | −48.154 | 8.599 | 13.275 |
| ATOM | 193 | HB | VAL | 52 | −48.377 | 9.756 | 11.442 |
| ATOM | 194 | HG23 | VAL | 52 | −48.14 | 7.546 | 13.596 |
| ATOM | 195 | HG22 | VAL | 52 | −48.998 | 9.103 | 13.77 |
| ATOM | 196 | HG21 | VAL | 52 | −47.226 | 9.072 | 13.622 |
| ATOM | 197 | HG13 | VAL | 52 | −49.788 | 8.091 | 10.214 |
| ATOM | 198 | HG12 | VAL | 52 | −50.477 | 8.438 | 11.818 |
| ATOM | 199 | HG11 | VAL | 52 | −49.574 | 6.921 | 11.551 |
| ATOM | 200 | H | VAL | 52 | −45.867 | 9.75 | 11.59 |
| ATOM | 201 | HA | VAL | 52 | −46.92 | 7.051 | 11.412 |
| ATOM | 202 | N | THR | 53 | −47.202 | 9.101 | 8.773 |
| ATOM | 203 | CA | THR | 53 | −47.413 | 9.007 | 7.329 |
| ATOM | 204 | C | THR | 53 | −46.41 | 8.068 | 6.693 |
| ATOM | 205 | O | THR | 53 | −46.823 | 7.159 | 5.989 |
| ATOM | 206 | CB | THR | 53 | −47.343 | 10.405 | 6.655 |
| ATOM | 207 | OG1 | THR | 53 | −48.317 | 11.241 | 7.307 |
| ATOM | 208 | CG2 | THR | 53 | −47.635 | 10.318 | 5.133 |
| ATOM | 209 | HB | THR | 53 | −46.337 | 10.832 | 6.805 |
| ATOM | 210 | HG22 | THR | 53 | −47.6 | 11.323 | 4.684 |
| ATOM | 211 | HG21 | THR | 53 | −48.636 | 9.894 | 4.963 |
| ATOM | 212 | HG23 | THR | 53 | −46.891 | 9.687 | 4.623 |
| ATOM | 213 | HG1 | THR | 53 | −48.315 | 12.139 | 6.99 |
| ATOM | 214 | H | THR | 53 | −47.019 | 9.999 | 9.184 |
| ATOM | 215 | HA | THR | 53 | −48.429 | 8.612 | 7.165 |
| ATOM | 216 | N | LEU | 54 | −45.09 | 8.268 | 6.915 |
| ATOM | 217 | CA | LEU | 54 | −44.099 | 7.417 | 6.255 |
| ATOM | 218 | C | LEU | 54 | −44.276 | 5.991 | 6.722 |
| ATOM | 219 | O | LEU | 54 | −44.28 | 5.088 | 5.901 |
| ATOM | 220 | CB | LEU | 54 | −42.621 | 7.828 | 6.522 |
| ATOM | 221 | CG | LEU | 54 | −42.221 | 9.253 | 6.028 |
| ATOM | 222 | CD1 | LEU | 54 | −40.76 | 9.569 | 6.463 |
| ATOM | 223 | CD2 | LEU | 54 | −42.344 | 9.403 | 4.486 |
| ATOM | 224 | H | LEU | 54 | −44.783 | 8.983 | 7.545 |
| ATOM | 225 | HB2 | LEU | 54 | −41.958 | 7.093 | 6.034 |
| ATOM | 226 | HB1 | LEU | 54 | −42.443 | 7.763 | 7.606 |
| ATOM | 227 | HG | LEU | 54 | −42.878 | 10.003 | 6.494 |
| ATOM | 228 | HD13 | LEU | 54 | −40.654 | 9.497 | 7.556 |
| ATOM | 229 | HD12 | LEU | 54 | −40.473 | 10.588 | 6.161 |
| ATOM | 230 | HD11 | LEU | 54 | −40.062 | 8.859 | 5.995 |
| ATOM | 231 | HD23 | LEU | 54 | −41.768 | 8.614 | 3.981 |
| ATOM | 232 | HD22 | LEU | 54 | −41.951 | 10.38 | 4.162 |
| ATOM | 233 | HD21 | LEU | 54 | −43.393 | 9.347 | 4.159 |
| ATOM | 234 | HA | LEU | 54 | −44.276 | 7.443 | 5.171 |
| ATOM | 235 | N | GLY | 55 | −44.413 | 5.764 | 8.045 |
| ATOM | 236 | CA | GLY | 55 | −44.529 | 4.401 | 8.552 |
| ATOM | 237 | C | GLY | 55 | −45.79 | 3.727 | 8.067 |
| ATOM | 238 | O | GLY | 55 | −45.753 | 2.548 | 7.748 |
| ATOM | 239 | H | GLY | 55 | −44.433 | 6.52 | 8.7 |
| ATOM | 240 | HA1 | GLY | 55 | −43.638 | 3.836 | 8.241 |
| ATOM | 241 | HA2 | GLY | 55 | −44.562 | 4.392 | 9.653 |
| ATOM | 242 | N | VAL | 56 | −46.932 | 4.448 | 8.021 |
| ATOM | 243 | CA | VAL | 56 | −48.17 | 3.818 | 7.569 |
| ATOM | 244 | C | VAL | 56 | −48.056 | 3.546 | 6.09 |
| ATOM | 245 | O | VAL | 56 | −48.195 | 2.395 | 5.704 |
| ATOM | 246 | CB | VAL | 56 | −49.441 | 4.659 | 7.896 |
| ATOM | 247 | CG1 | VAL | 56 | −50.691 | 4.127 | 7.14 |
| ATOM | 248 | CG2 | VAL | 56 | −49.712 | 4.647 | 9.427 |
| ATOM | 249 | HB | VAL | 56 | −49.279 | 5.701 | 7.568 |
| ATOM | 250 | HG23 | VAL | 56 | −48.82 | 4.958 | 9.991 |
| ATOM | 251 | HG22 | VAL | 56 | −49.99 | 3.635 | 9.76 |
| ATOM | 252 | HG21 | VAL | 56 | −50.538 | 5.333 | 9.674 |
| ATOM | 253 | HG13 | VAL | 56 | −50.581 | 4.267 | 6.054 |
| ATOM | 254 | HG12 | VAL | 56 | −51.593 | 4.672 | 7.458 |
| ATOM | 255 | HG11 | VAL | 56 | −50.838 | 3.055 | 7.345 |
| ATOM | 256 | H | VAL | 56 | −46.94 | 5.419 | 8.27 |
| ATOM | 257 | HA | VAL | 56 | −48.285 | 2.848 | 8.081 |
| ATOM | 258 | N | ILE | 57 | −47.809 | 4.566 | 5.235 |
| ATOM | 259 | CA | ILE | 57 | −47.762 | 4.282 | 3.799 |
| ATOM | 260 | C | ILE | 57 | −46.679 | 3.25 | 3.558 |
| ATOM | 261 | O | ILE | 57 | −46.878 | 2.347 | 2.762 |
| ATOM | 262 | CB | ILE | 57 | −47.659 | 5.553 | 2.898 |
| ATOM | 263 | CG1 | ILE | 57 | −48.193 | 5.264 | 1.462 |
| ATOM | 264 | CG2 | ILE | 57 | −46.205 | 6.084 | 2.85 |
| ATOM | 265 | CD1 | ILE | 57 | −48.135 | 6.485 | 0.506 |
| ATOM | 266 | HB | ILE | 57 | −48.305 | 6.332 | 3.339 |
| ATOM | 267 | HG23 | ILE | 57 | −45.802 | 6.16 | 3.865 |
| ATOM | 268 | HG22 | ILE | 57 | −46.151 | 7.086 | 2.399 |
| ATOM | 269 | HG21 | ILE | 57 | −45.581 | 5.392 | 2.265 |
| ATOM | 270 | HG12 | ILE | 57 | −49.246 | 4.94 | 1.525 |
| ATOM | 271 | HG11 | ILE | 57 | −47.61 | 4.445 | 1.018 |
| ATOM | 272 | HD13 | ILE | 57 | −48.622 | 7.358 | 0.966 |
| ATOM | 273 | HD12 | ILE | 57 | −48.659 | 6.247 | −0.434 |
| ATOM | 274 | HD11 | ILE | 57 | −47.097 | 6.744 | 0.249 |
| ATOM | 275 | H | ILE | 57 | −47.648 | 5.5 | 5.565 |
| ATOM | 276 | HA | ILE | 57 | −48.727 | 3.811 | 3.561 |
| ATOM | 277 | N | SER | 58 | −45.528 | 3.327 | 4.266 |
| ATOM | 278 | CA | SER | 58 | −44.539 | 2.262 | 4.143 |
| ATOM | 279 | C | SER | 58 | −45.19 | 0.94 | 4.454 |
| ATOM | 280 | O | SER | 58 | −44.97 | −0.006 | 3.716 |
| ATOM | 281 | CB | SER | 58 | −43.371 | 2.377 | 5.157 |
| ATOM | 282 | OG | SER | 58 | −42.617 | 1.15 | 5.16 |
| ATOM | 283 | HB2 | SER | 58 | −42.738 | 3.238 | 4.897 |
| ATOM | 284 | HB1 | SER | 58 | −43.782 | 2.532 | 6.164 |
| ATOM | 285 | H | SER | 58 | −45.352 | 4.067 | 4.917 |
| ATOM | 286 | HG | SER | 58 | −41.933 | 1.11 | 5.82 |
| ATOM | 287 | HA | SER | 58 | −44.133 | 2.249 | 3.116 |
| ATOM | 288 | N | LEU | 59 | −45.967 | 0.824 | 5.552 |
| ATOM | 289 | CA | LEU | 59 | −46.504 | −0.486 | 5.894 |
| ATOM | 290 | C | LEU | 59 | −47.42 | −0.971 | 4.795 |
| ATOM | 291 | O | LEU | 59 | −47.186 | −2.067 | 4.308 |
| ATOM | 292 | CB | LEU | 59 | −47.237 | −0.499 | 7.264 |
| ATOM | 293 | CG | LEU | 59 | −47.848 | −1.883 | 7.645 |
| ATOM | 294 | CD1 | LEU | 59 | −46.777 | −3.01 | 7.727 |
| ATOM | 295 | CD2 | LEU | 59 | −48.587 | −1.765 | 9.007 |
| ATOM | 296 | HB2 | LEU | 59 | −46.526 | −0.188 | 8.045 |
| ATOM | 297 | HB1 | LEU | 59 | −48.047 | 0.246 | 7.229 |
| ATOM | 298 | HG | LEU | 59 | −48.594 | −2.171 | 6.884 |
| ATOM | 299 | HD13 | LEU | 59 | −46.351 | −3.238 | 6.74 |
| ATOM | 300 | HD12 | LEU | 59 | −47.229 | −3.94 | 8.107 |
| ATOM | 301 | HD11 | LEU | 59 | −45.962 | −2.717 | 8.406 |
| ATOM | 302 | HD23 | LEU | 59 | −47.878 | −1.499 | 9.807 |
| ATOM | 303 | HD22 | LEU | 59 | −49.07 | −2.718 | 9.272 |
| ATOM | 304 | HD21 | LEU | 59 | −49.367 | −0.989 | 8.959 |
| ATOM | 305 | H | LEU | 59 | −46.162 | 1.606 | 6.148 |
| ATOM | 306 | HA | LEU | 59 | −45.636 | −1.161 | 5.968 |
| ATOM | 307 | N | LEU | 60 | −48.465 | −0.206 | 4.385 |
| ATOM | 308 | CA | LEU | 60 | −49.372 | −0.745 | 3.368 |
| ATOM | 309 | C | LEU | 60 | −48.66 | −0.951 | 2.044 |
| ATOM | 310 | O | LEU | 60 | −49.099 | −1.81 | 1.296 |
| ATOM | 311 | CB | LEU | 60 | −50.85 | −0.238 | 3.235 |
| ATOM | 312 | CG | LEU | 60 | −51.163 | 1.212 | 2.743 |
| ATOM | 313 | CD1 | LEU | 60 | −51.258 | 2.235 | 3.908 |
| ATOM | 314 | CD2 | LEU | 60 | −50.202 | 1.695 | 1.629 |
| ATOM | 315 | HA | LEU | 60 | −49.565 | −1.767 | 3.731 |
| ATOM | 316 | HB2 | LEU | 60 | −51.333 | −0.918 | 2.509 |
| ATOM | 317 | HB1 | LEU | 60 | −51.364 | −0.384 | 4.2 |
| ATOM | 318 | HG | LEU | 60 | −52.176 | 1.202 | 2.297 |
| ATOM | 319 | HD13 | LEU | 60 | −52.069 | 1.964 | 4.602 |
| ATOM | 320 | HD12 | LEU | 60 | −51.46 | 3.247 | 3.523 |
| ATOM | 321 | HD11 | LEU | 60 | −50.325 | 2.26 | 4.475 |
| ATOM | 322 | HD23 | LEU | 60 | −49.205 | 1.839 | 2.054 |
| ATOM | 323 | HD22 | LEU | 60 | −50.54 | 2.656 | 1.212 |
| ATOM | 324 | HD21 | LEU | 60 | −50.149 | 0.962 | 0.809 |
| ATOM | 325 | H | LEU | 60 | −48.608 | 0.713 | 4.752 |
| ATOM | 326 | N | GLU | 61 | −47.568 | −0.221 | 1.709 |
| ATOM | 327 | CA | GLU | 61 | −46.854 | −0.514 | 0.461 |
| ATOM | 328 | C | GLU | 61 | −45.97 | −1.726 | 0.658 |
| ATOM | 329 | O | GLU | 61 | −46.066 | −2.676 | −0.103 |
| ATOM | 330 | CB | GLU | 61 | −45.962 | 0.663 | −0.025 |
| ATOM | 331 | CG | GLU | 61 | −46.797 | 1.867 | −0.529 |
| ATOM | 332 | CD | GLU | 61 | −45.884 | 3.007 | −0.902 |
| ATOM | 333 | OE1 | GLU | 61 | −45.245 | 3.584 | 0.02 |
| ATOM | 334 | OE2 | GLU | 61 | −45.795 | 3.334 | −2.116 |
| ATOM | 335 | HB2 | GLU | 61 | −45.287 | 0.99 | 0.782 |
| ATOM | 336 | HB1 | GLU | 61 | −45.345 | 0.318 | −0.868 |
| ATOM | 337 | HG2 | GLU | 61 | −47.488 | 2.194 | 0.257 |
| ATOM | 338 | HG1 | GLU | 61 | −47.399 | 1.571 | −1.403 |
| ATOM | 339 | H | GLU | 61 | −47.195 | 0.483 | 2.319 |
| ATOM | 340 | HA | GLU | 61 | −47.573 | −0.733 | −0.346 |
| ATOM | 341 | N | ASN | 62 | −45.084 | −1.714 | 1.677 |
| ATOM | 342 | CA | ASN | 62 | −44.163 | −2.831 | 1.856 |
| ATOM | 343 | C | ASN | 62 | −44.914 | −4.129 | 2.032 |
| ATOM | 344 | O | ASN | 62 | −44.55 | −5.105 | 1.393 |
| ATOM | 345 | CB | ASN | 62 | −43.175 | −2.629 | 3.032 |
| ATOM | 346 | CG | ASN | 62 | −42.154 | −1.591 | 2.636 |
| ATOM | 347 | OD1 | ASN | 62 | −41.3 | −1.919 | 1.831 |
| ATOM | 348 | ND2 | ASN | 62 | −42.193 | −0.343 | 3.136 |
| ATOM | 349 | H | ASN | 62 | −45.03 | −0.94 | 2.306 |
| ATOM | 350 | HB2 | ASN | 62 | −42.631 | −3.568 | 3.211 |
| ATOM | 351 | HB1 | ASN | 62 | −43.716 | −2.366 | 3.954 |
| ATOM | 352 | HD21 | ASN | 62 | −42.892 | −0.081 | 3.797 |
| ATOM | 353 | HD22 | ASN | 62 | −41.515 | 0.338 | 2.848 |
| ATOM | 354 | HA | ASN | 62 | −43.555 | −2.905 | 0.944 |
| ATOM | 355 | N | ILE | 63 | −45.964 | −4.176 | 2.886 |
| ATOM | 356 | CA | ILE | 63 | −46.688 | −5.438 | 3.043 |
| ATOM | 357 | C | ILE | 63 | −47.291 | −5.839 | 1.71 |
| ATOM | 358 | O | ILE | 63 | −47.398 | −7.022 | 1.434 |
| ATOM | 359 | CB | ILE | 63 | −47.779 | −5.424 | 4.159 |
| ATOM | 360 | CG1 | ILE | 63 | −48.151 | −6.847 | 4.683 |
| ATOM | 361 | CG2 | ILE | 63 | −49.07 | −4.729 | 3.646 |
| ATOM | 362 | CD1 | ILE | 63 | −47.034 | −7.551 | 5.505 |
| ATOM | 363 | HB | ILE | 63 | −47.385 | −4.846 | 5.012 |
| ATOM | 364 | HG23 | ILE | 63 | −48.805 | −3.806 | 3.124 |
| ATOM | 365 | HG22 | ILE | 63 | −49.742 | −4.482 | 4.483 |
| ATOM | 366 | HG21 | ILE | 63 | −49.617 | −5.373 | 2.94 |
| ATOM | 367 | HG12 | ILE | 63 | −48.445 | −7.492 | 3.841 |
| ATOM | 368 | HG11 | ILE | 63 | −49.023 | −6.75 | 5.352 |
| ATOM | 369 | HD13 | ILE | 63 | −46.171 | −7.836 | 4.886 |
| ATOM | 370 | HD12 | ILE | 63 | −47.435 | −8.474 | 5.952 |
| ATOM | 371 | HD11 | ILE | 63 | −46.689 | −6.896 | 6.319 |
| ATOM | 372 | H | ILE | 63 | −46.262 | −3.363 | 3.395 |
| ATOM | 373 | HA | ILE | 63 | −45.926 | −6.162 | 3.349 |
| ATOM | 374 | N | LEU | 64 | −47.706 | −4.878 | 0.853 |
| ATOM | 375 | CA | LEU | 64 | −48.315 | −5.255 | −0.42 |
| ATOM | 376 | C | LEU | 64 | −47.241 | −5.836 | −1.305 |
| ATOM | 377 | O | LEU | 64 | −47.408 | −6.956 | −1.764 |
| ATOM | 378 | CB | LEU | 64 | −49.04 | −4.035 | −1.064 |
| ATOM | 379 | CG | LEU | 64 | −49.834 | −4.317 | −2.379 |
| ATOM | 380 | CD1 | LEU | 64 | −50.78 | −3.116 | −2.673 |
| ATOM | 381 | CD2 | LEU | 64 | −48.913 | −4.541 | −3.614 |
| ATOM | 382 | H | LEU | 64 | −47.585 | −3.903 | 1.051 |
| ATOM | 383 | HB2 | LEU | 64 | −48.327 | −3.218 | −1.247 |
| ATOM | 384 | HB1 | LEU | 64 | −49.758 | −3.689 | −0.304 |
| ATOM | 385 | HG | LEU | 64 | −50.462 | −5.213 | −2.236 |
| ATOM | 386 | HD13 | LEU | 64 | −51.483 | −2.955 | −1.84 |
| ATOM | 387 | HD12 | LEU | 64 | −51.374 | −3.299 | −3.582 |
| ATOM | 388 | HD11 | LEU | 64 | −50.193 | −2.196 | −2.817 |
| ATOM | 389 | HD23 | LEU | 64 | −48.186 | −3.72 | −3.709 |
| ATOM | 390 | HD22 | LEU | 64 | −49.512 | −4.58 | −4.537 |
| ATOM | 391 | HD21 | LEU | 64 | −48.367 | −5.489 | −3.552 |
| ATOM | 392 | HA | LEU | 64 | −49.08 | −6.028 | −0.234 |
| ATOM | 393 | N | VAL | 65 | −46.134 | −5.109 | −1.577 |
| ATOM | 394 | CA | VAL | 65 | −45.175 | −5.626 | −2.554 |
| ATOM | 395 | C | VAL | 65 | −44.603 | −6.934 | −2.058 |
| ATOM | 396 | O | VAL | 65 | −44.466 | −7.841 | −2.862 |
| ATOM | 397 | CB | VAL | 65 | −44.046 | −4.652 | −3.018 |
| ATOM | 398 | CG1 | VAL | 65 | −44.63 | −3.301 | −3.526 |
| ATOM | 399 | CG2 | VAL | 65 | −42.972 | −4.415 | −1.925 |
| ATOM | 400 | HB | VAL | 65 | −43.522 | −5.127 | −3.867 |
| ATOM | 401 | HG23 | VAL | 65 | −43.46 | −4.022 | −1.03 |
| ATOM | 402 | HG22 | VAL | 65 | −42.219 | −3.687 | −2.266 |
| ATOM | 403 | HG21 | VAL | 65 | −42.449 | −5.348 | −1.664 |
| ATOM | 404 | HG13 | VAL | 65 | −45.368 | −3.48 | −4.323 |
| ATOM | 405 | HG12 | VAL | 65 | −43.832 | −2.664 | −3.935 |
| ATOM | 406 | HG11 | VAL | 65 | −45.119 | −2.739 | −2.719 |
| ATOM | 407 | H | VAL | 65 | −45.974 | −4.229 | −1.124 |
| ATOM | 408 | HA | VAL | 65 | −45.756 | −5.85 | −3.465 |
| ATOM | 409 | N | ILE | 66 | −44.262 | −7.086 | −0.757 |
| ATOM | 410 | CA | ILE | 66 | −43.693 | −8.365 | −0.322 |
| ATOM | 411 | C | ILE | 66 | −44.707 | −9.473 | −0.507 |
| ATOM | 412 | O | ILE | 66 | −44.314 | −10.57 | −0.873 |
| ATOM | 413 | CB | ILE | 66 | −43.147 | −8.35 | 1.139 |
| ATOM | 414 | CG1 | ILE | 66 | −42.135 | −9.509 | 1.386 |
| ATOM | 415 | CG2 | ILE | 66 | −44.296 | −8.394 | 2.178 |
| ATOM | 416 | CD1 | ILE | 66 | −41.508 | −9.48 | 2.806 |
| ATOM | 417 | HB | ILE | 66 | −42.604 | −7.401 | 1.262 |
| ATOM | 418 | HG23 | ILE | 66 | −45.07 | −7.688 | 1.876 |
| ATOM | 419 | HG22 | ILE | 66 | −43.95 | −8.096 | 3.177 |
| ATOM | 420 | HG21 | ILE | 66 | −44.742 | −9.398 | 2.239 |
| ATOM | 421 | HG12 | ILE | 66 | −41.318 | −9.425 | 0.653 |
| ATOM | 422 | HG11 | ILE | 66 | −42.63 | −10.481 | 1.231 |
| ATOM | 423 | HD13 | ILE | 66 | −41.065 | −8.493 | 3.011 |
| ATOM | 424 | HD12 | ILE | 66 | −40.719 | −10.243 | 2.883 |
| ATOM | 425 | HD11 | ILE | 66 | −42.256 | −9.702 | 3.582 |
| ATOM | 426 | H | ILE | 66 | −44.396 | −6.345 | −0.092 |
| ATOM | 427 | HA | ILE | 66 | −42.836 | −8.559 | −0.986 |
| ATOM | 428 | N | VAL | 67 | −46.017 | −9.228 | −0.266 |
| ATOM | 429 | CA | VAL | 67 | −47.001 | −10.296 | −0.441 |
| ATOM | 430 | C | VAL | 67 | −47.161 | −10.562 | −1.92 |
| ATOM | 431 | O | VAL | 67 | −47.109 | −11.715 | −2.319 |
| ATOM | 432 | CB | VAL | 67 | −48.367 | −9.965 | 0.234 |
| ATOM | 433 | CG1 | VAL | 67 | −49.482 | −10.95 | −0.219 |
| ATOM | 434 | CG2 | VAL | 67 | −48.227 | −10.017 | 1.782 |
| ATOM | 435 | HB | VAL | 67 | −48.678 | −8.95 | −0.068 |
| ATOM | 436 | HG23 | VAL | 67 | −48.048 | −11.051 | 2.118 |
| ATOM | 437 | HG22 | VAL | 67 | −49.147 | −9.651 | 2.262 |
| ATOM | 438 | HG21 | VAL | 67 | −47.386 | −9.401 | 2.129 |
| ATOM | 439 | HG13 | VAL | 67 | −49.685 | −10.852 | −1.297 |
| ATOM | 440 | HG12 | VAL | 67 | −50.419 | −10.74 | 0.32 |
| ATOM | 441 | HG11 | VAL | 67 | −49.183 | −11.989 | −0.011 |
| ATOM | 442 | H | VAL | 67 | −46.344 | −8.313 | −0.016 |
| ATOM | 443 | HA | VAL | 67 | −46.622 | −11.215 | 0.033 |
| ATOM | 444 | N | ALA | 68 | −47.355 | −9.519 | −2.756 |
| ATOM | 445 | CA | ALA | 68 | −47.473 | −9.756 | −4.195 |
| ATOM | 446 | C | ALA | 68 | −46.252 | −10.494 | −4.698 |
| ATOM | 447 | O | ALA | 68 | −46.393 | −11.403 | −5.501 |
| ATOM | 448 | CB | ALA | 68 | −47.611 | −8.425 | −4.978 |
| ATOM | 449 | HB3 | ALA | 68 | −46.727 | −7.791 | −4.814 |
| ATOM | 450 | HB2 | ALA | 68 | −48.507 | −7.881 | −4.642 |
| ATOM | 451 | HB1 | ALA | 68 | −47.705 | −8.626 | −6.057 |
| ATOM | 452 | H | ALA | 68 | −47.403 | −8.581 | −2.407 |
| ATOM | 453 | HA | ALA | 68 | −48.369 | −10.372 | −4.378 |
| ATOM | 454 | N | ILE | 69 | −45.042 | −10.12 | −4.229 |
| ATOM | 455 | CA | ILE | 69 | −43.842 | −10.86 | −4.621 |
| ATOM | 456 | C | ILE | 69 | −43.965 | −12.281 | −4.12 |
| ATOM | 457 | O | ILE | 69 | −43.711 | −13.201 | −4.881 |
| ATOM | 458 | CB | ILE | 69 | −42.542 | −10.179 | −4.088 |
| ATOM | 459 | CG1 | ILE | 69 | −42.269 | −8.87 | −4.896 |
| ATOM | 460 | CG2 | ILE | 69 | −41.342 | −11.171 | −4.125 |
| ATOM | 461 | CD1 | ILE | 69 | −41.234 | −7.92 | −4.231 |
| ATOM | 462 | HB | ILE | 69 | −42.696 | −9.921 | −3.029 |
| ATOM | 463 | HG23 | ILE | 69 | −41.507 | −12.015 | −3.438 |
| ATOM | 464 | HG22 | ILE | 69 | −40.405 | −10.691 | −3.812 |
| ATOM | 465 | HG21 | ILE | 69 | −41.219 | −11.578 | −5.14 |
| ATOM | 466 | HG12 | ILE | 69 | −43.204 | −8.297 | −5 |
| ATOM | 467 | HG11 | ILE | 69 | −41.942 | −9.125 | −5.916 |
| ATOM | 468 | HD13 | ILE | 69 | −41.573 | −7.632 | −3.224 |
| ATOM | 469 | HD12 | ILE | 69 | −41.129 | −7.005 | −4.835 |
| ATOM | 470 | HD11 | ILE | 69 | −40.241 | −8.383 | −4.147 |
| ATOM | 471 | H | ILE | 69 | −44.962 | −9.366 | −3.574 |
| ATOM | 472 | HA | ILE | 69 | −43.786 | −10.898 | −5.722 |
| ATOM | 473 | N | ALA | 70 | −44.34 | −12.494 | −2.84 |
| ATOM | 474 | CA | ALA | 70 | −44.462 | −13.862 | −2.343 |
| ATOM | 475 | C | ALA | 70 | −45.554 | −14.568 | −3.106 |
| ATOM | 476 | O | ALA | 70 | −45.419 | −15.731 | −3.454 |
| ATOM | 477 | CB | ALA | 70 | −44.796 | −13.899 | −0.83 |
| ATOM | 478 | HB3 | ALA | 70 | −45.775 | −13.434 | −0.645 |
| ATOM | 479 | HB2 | ALA | 70 | −44.028 | −13.354 | −0.258 |
| ATOM | 480 | HB1 | ALA | 70 | −44.831 | −14.94 | −0.473 |
| ATOM | 481 | H | ALA | 70 | −44.549 | −11.729 | −2.226 |
| ATOM | 482 | HA | ALA | 70 | −43.505 | −14.387 | −2.503 |
| ATOM | 483 | H3 | ALA | 70 | −46.477 | −14.041 | −3.346 |
| ATOM | 484 | N | PRO | 78 | −33.837 | −12.212 | −9.397 |
| ATOM | 485 | CA | PRO | 78 | −33.141 | −11.409 | −8.403 |
| ATOM | 486 | C | PRO | 78 | −33.859 | −10.111 | −8.114 |
| ATOM | 487 | O | PRO | 78 | −33.965 | −9.749 | −6.953 |
| ATOM | 488 | CB | PRO | 78 | −31.767 | −11.25 | −9.101 |
| ATOM | 489 | CG | PRO | 78 | −31.646 | −12.521 | −9.975 |
| ATOM | 490 | CD | PRO | 78 | −33.077 | −12.717 | −10.526 |
| ATOM | 491 | HB2 | PRO | 78 | −30.936 | −11.173 | −8.383 |
| ATOM | 492 | HB1 | PRO | 78 | −31.766 | −10.363 | −9.755 |
| ATOM | 493 | HG1 | PRO | 78 | −30.886 | −12.429 | −10.768 |
| ATOM | 494 | HG2 | PRO | 78 | −31.388 | −13.377 | −9.328 |
| ATOM | 495 | HD1 | PRO | 78 | −33.256 | −12.082 | −11.409 |
| ATOM | 496 | HD2 | PRO | 78 | −33.302 | −13.769 | −10.766 |
| ATOM | 497 | HA | PRO | 78 | −33.022 | −11.995 | −7.481 |
| ATOM | 498 | H4 | PRO | 78 | −34.819 | −12.399 | −9.314 |
| ATOM | 499 | N | MET | 79 | −34.364 | −9.422 | −9.166 |
| ATOM | 500 | CA | MET | 79 | −35.055 | −8.143 | −8.98 |
| ATOM | 501 | C | MET | 79 | −35.919 | −8.151 | −7.737 |
| ATOM | 502 | O | MET | 79 | −35.828 | −7.249 | −6.92 |
| ATOM | 503 | CB | MET | 79 | −35.946 | −7.79 | −10.206 |
| ATOM | 504 | CG | MET | 79 | −36.547 | −6.361 | −10.116 |
| ATOM | 505 | SD | MET | 79 | −35.217 | −5.12 | −10.308 |
| ATOM | 506 | CE | MET | 79 | −35.374 | −4.149 | −8.776 |
| ATOM | 507 | HB2 | MET | 79 | −35.348 | −7.849 | −11.13 |
| ATOM | 508 | HB1 | MET | 79 | −36.76 | −8.528 | −10.288 |
| ATOM | 509 | HG2 | MET | 79 | −37.091 | −6.211 | −9.172 |
| ATOM | 510 | HG1 | MET | 79 | −37.258 | −6.214 | −10.943 |
| ATOM | 511 | HE3 | MET | 79 | −35.292 | −4.806 | −7.897 |
| ATOM | 512 | HE2 | MET | 79 | −36.347 | −3.636 | −8.766 |
| ATOM | 513 | HE1 | MET | 79 | −34.571 | −3.399 | −8.738 |
| ATOM | 514 | H | MET | 79 | −34.233 | −9.764 | −10.098 |
| ATOM | 515 | HA | MET | 79 | −34.293 | −7.358 | −8.907 |
| ATOM | 516 | LPD2 | MET | 79 | −35.319 | −4.733 | −10.856 |
| ATOM | 517 | LPD1 | MET | 79 | −34.608 | −5.417 | −10.35 |
| ATOM | 518 | N | TYR | 80 | −36.778 | −9.182 | −7.591 |
| ATOM | 519 | CA | TYR | 80 | −37.722 | −9.207 | −6.475 |
| ATOM | 520 | C | TYR | 80 | −37.081 | −9.696 | −5.196 |
| ATOM | 521 | O | TYR | 80 | −37.416 | −9.191 | −4.135 |
| ATOM | 522 | CB | TYR | 80 | −38.919 | −10.122 | −6.84 |
| ATOM | 523 | CG | TYR | 80 | −39.509 | −9.875 | −8.241 |
| ATOM | 524 | CD1 | TYR | 80 | −39.392 | −8.649 | −8.908 |
| ATOM | 525 | CD2 | TYR | 80 | −40.182 | −10.924 | −8.878 |
| ATOM | 526 | CE1 | TYR | 80 | −39.818 | −8.517 | −10.233 |
| ATOM | 527 | CE2 | TYR | 80 | −40.652 | −10.779 | −10.187 |
| ATOM | 528 | CZ | TYR | 80 | −40.458 | −9.581 | −10.879 |
| ATOM | 529 | OH | TYR | 80 | −40.907 | −9.473 | −12.198 |
| ATOM | 530 | HB1 | TYR | 80 | −38.579 | −11.168 | −6.78 |
| ATOM | 531 | HB2 | TYR | 80 | −39.712 | −9.975 | −6.101 |
| ATOM | 532 | HD1 | TYR | 80 | −38.972 | −7.782 | −8.413 |
| ATOM | 533 | HD2 | TYR | 80 | −40.346 | −11.863 | −8.359 |
| ATOM | 534 | HE1 | TYR | 80 | −39.649 | −7.582 | −10.756 |
| ATOM | 535 | HE2 | TYR | 80 | −41.17 | −11.599 | −10.672 |
| ATOM | 536 | HH | TYR | 80 | −40.8 | −8.61 | −12.582 |
| ATOM | 537 | H | TYR | 80 | −36.779 | −9.942 | −8.244 |
| ATOM | 538 | HA | TYR | 80 | −38.11 | −8.196 | −6.279 |
| ATOM | 539 | N | PHE | 81 | −36.159 | −10.681 | −5.26 |
| ATOM | 540 | CA | PHE | 81 | −35.525 | −11.163 | −4.034 |
| ATOM | 541 | C | PHE | 81 | −34.827 | −10.021 | −3.335 |
| ATOM | 542 | O | PHE | 81 | −34.84 | −9.988 | −2.114 |
| ATOM | 543 | CB | PHE | 81 | −34.494 | −12.291 | −4.296 |
| ATOM | 544 | CG | PHE | 81 | −35.217 | −13.585 | −4.701 |
| ATOM | 545 | CD1 | PHE | 81 | −35.677 | −14.46 | −3.709 |
| ATOM | 546 | CD2 | PHE | 81 | −35.425 | −13.911 | −6.046 |
| ATOM | 547 | CE1 | PHE | 81 | −36.322 | −15.65 | −4.056 |
| ATOM | 548 | CE2 | PHE | 81 | −36.074 | −15.098 | −6.396 |
| ATOM | 549 | CZ | PHE | 81 | −36.521 | −15.972 | −5.401 |
| ATOM | 550 | HB2 | PHE | 81 | −33.776 | −11.976 | −5.069 |
| ATOM | 551 | HB1 | PHE | 81 | −33.92 | −12.483 | −3.375 |
| ATOM | 552 | HD1 | PHE | 81 | −35.535 | −14.218 | −2.66 |
| ATOM | 553 | HD2 | PHE | 81 | −35.086 | −13.247 | −6.831 |
| ATOM | 554 | HE1 | PHE | 81 | −36.67 | −16.325 | −3.28 |
| ATOM | 555 | HE2 | PHE | 81 | −36.23 | −15.343 | −7.443 |
| ATOM | 556 | HZ | PHE | 81 | −37.02 | −16.897 | −5.672 |
| ATOM | 557 | H | PHE | 81 | −35.88 | −11.065 | −6.139 |
| ATOM | 558 | HA | PHE | 81 | −36.307 | −11.551 | −3.36 |
| ATOM | 559 | N | PHE | 82 | −34.212 | −9.066 | −4.066 |
| ATOM | 560 | CA | PHE | 82 | −33.611 | −7.936 | −3.363 |
| ATOM | 561 | C | PHE | 82 | −34.71 | −7.205 | −2.618 |
| ATOM | 562 | O | PHE | 82 | −34.586 | −6.972 | −1.425 |
| ATOM | 563 | CB | PHE | 82 | −32.907 | −6.914 | −4.297 |
| ATOM | 564 | CG | PHE | 82 | −32.079 | −7.58 | −5.406 |
| ATOM | 565 | CD1 | PHE | 82 | −31.01 | −8.427 | −5.096 |
| ATOM | 566 | CD2 | PHE | 82 | −32.386 | −7.332 | −6.748 |
| ATOM | 567 | CE1 | PHE | 82 | −30.215 | −8.958 | −6.117 |
| ATOM | 568 | CE2 | PHE | 82 | −31.606 | −7.878 | −7.772 |
| ATOM | 569 | CZ | PHE | 82 | −30.488 | −8.648 | −7.452 |
| ATOM | 570 | HB2 | PHE | 82 | −33.654 | −6.246 | −4.754 |
| ATOM | 571 | HB1 | PHE | 82 | −32.239 | −6.28 | −3.699 |
| ATOM | 572 | HD1 | PHE | 82 | −30.791 | −8.667 | −4.061 |
| ATOM | 573 | HD2 | PHE | 82 | −33.225 | −6.697 | −6.996 |
| ATOM | 574 | HE1 | PHE | 82 | −29.381 | −9.61 | −5.884 |
| ATOM | 575 | HE2 | PHE | 82 | −31.855 | −7.7 | −8.813 |
| ATOM | 576 | HZ | PHE | 82 | −29.832 | −9.002 | −8.24 |
| ATOM | 577 | H | PHE | 82 | −34.188 | −9.103 | −5.07 |
| ATOM | 578 | HA | PHE | 82 | −32.872 | −8.328 | −2.643 |
| ATOM | 579 | N | ILE | 83 | −35.799 | −6.84 | −3.334 |
| ATOM | 580 | CA | ILE | 83 | −36.863 | −6.042 | −2.72 |
| ATOM | 581 | C | ILE | 83 | −37.411 | −6.766 | −1.519 |
| ATOM | 582 | O | ILE | 83 | −37.676 | −6.114 | −0.521 |
| ATOM | 583 | CB | ILE | 83 | −37.99 | −5.707 | −3.74 |
| ATOM | 584 | CG1 | ILE | 83 | −37.441 | −4.727 | −4.824 |
| ATOM | 585 | CG2 | ILE | 83 | −39.241 | −5.109 | −3.028 |
| ATOM | 586 | CD1 | ILE | 83 | −38.196 | −4.868 | −6.168 |
| ATOM | 587 | HB | ILE | 83 | −38.295 | −6.651 | −4.218 |
| ATOM | 588 | HG23 | ILE | 83 | −39.703 | −5.846 | −2.353 |
| ATOM | 589 | HG22 | ILE | 83 | −40.01 | −4.801 | −3.751 |
| ATOM | 590 | HG21 | ILE | 83 | −38.956 | −4.227 | −2.433 |
| ATOM | 591 | HG12 | ILE | 83 | −36.375 | −4.905 | −5.039 |
| ATOM | 592 | HG11 | ILE | 83 | −37.526 | −3.691 | −4.454 |
| ATOM | 593 | HD13 | ILE | 83 | −37.952 | −5.818 | −6.661 |
| ATOM | 594 | HD12 | ILE | 83 | −37.913 | −4.05 | −6.844 |
| ATOM | 595 | HD11 | ILE | 83 | −39.278 | −4.847 | −6 |
| ATOM | 596 | H | ILE | 83 | −35.895 | −7.125 | −4.291 |
| ATOM | 597 | HA | ILE | 83 | −36.442 | −5.088 | −2.37 |
| ATOM | 598 | N | CYS | 84 | −37.585 | −8.103 | −1.579 |
| ATOM | 599 | CA | CYS | 84 | −38.047 | −8.82 | −0.393 |
| ATOM | 600 | C | CYS | 84 | −37.251 | −8.385 | 0.818 |
| ATOM | 601 | O | CYS | 84 | −37.838 | −8.124 | 1.856 |
| ATOM | 602 | CB | CYS | 84 | −37.897 | −10.354 | −0.577 |
| ATOM | 603 | SG | CYS | 84 | −38.424 | −11.251 | 0.92 |
| ATOM | 604 | HG | CYS | 84 | −39.408 | −11.045 | 0.873 |
| ATOM | 605 | HB2 | CYS | 84 | −38.489 | −10.699 | −1.441 |
| ATOM | 606 | HB1 | CYS | 84 | −36.843 | −10.613 | −0.75 |
| ATOM | 607 | H | CYS | 84 | −37.398 | −8.611 | −2.425 |
| ATOM | 608 | HA | CYS | 84 | −39.11 | −8.575 | −0.236 |
| ATOM | 609 | LPG2 | CYS | 84 | −38.137 | −11.002 | 1.482 |
| ATOM | 610 | LPG1 | CYS | 84 | −38.299 | −11.917 | 0.877 |
| ATOM | 611 | N | SER | 85 | −35.907 | −8.29 | 0.709 |
| ATOM | 612 | CA | SER | 85 | −35.119 | −7.908 | 1.88 |
| ATOM | 613 | C | SER | 85 | −35.411 | −6.49 | 2.306 |
| ATOM | 614 | O | SER | 85 | −35.574 | −6.249 | 3.493 |
| ATOM | 615 | CB | SER | 85 | −33.593 | −8.008 | 1.637 |
| ATOM | 616 | OG | SER | 85 | −32.933 | −7.864 | 2.909 |
| ATOM | 617 | HB2 | SER | 85 | −33.37 | −8.989 | 1.193 |
| ATOM | 618 | HB1 | SER | 85 | −33.265 | −7.226 | 0.932 |
| ATOM | 619 | HG | SER | 85 | −31.985 | −7.94 | 2.867 |
| ATOM | 620 | H | SER | 85 | −35.439 | −8.464 | −0.162 |
| ATOM | 621 | HA | SER | 85 | −35.37 | −8.597 | 2.703 |
| ATOM | 622 | N | LEU | 86 | −35.472 | −5.523 | 1.361 |
| ATOM | 623 | CA | LEU | 86 | −35.716 | −4.145 | 1.78 |
| ATOM | 624 | C | LEU | 86 | −37.088 | −4.109 | 2.401 |
| ATOM | 625 | O | LEU | 86 | −37.228 | −3.573 | 3.486 |
| ATOM | 626 | CB | LEU | 86 | −35.616 | −3.063 | 0.663 |
| ATOM | 627 | CG | LEU | 86 | −34.21 | −2.886 | −0.011 |
| ATOM | 628 | CD1 | LEU | 86 | −33.049 | −3.479 | 0.832 |
| ATOM | 629 | CD2 | LEU | 86 | −34.154 | −3.446 | −1.46 |
| ATOM | 630 | H | LEU | 86 | −35.384 | −5.733 | 0.385 |
| ATOM | 631 | HB2 | LEU | 86 | −35.889 | −2.108 | 1.146 |
| ATOM | 632 | HB1 | LEU | 86 | −36.384 | −3.265 | −0.1 |
| ATOM | 633 | HG | LEU | 86 | −34.015 | −1.802 | −0.122 |
| ATOM | 634 | HD13 | LEU | 86 | −32.942 | −2.924 | 1.776 |
| ATOM | 635 | HD12 | LEU | 86 | −32.108 | −3.405 | 0.281 |
| ATOM | 636 | HD11 | LEU | 86 | −33.193 | −4.547 | 1.042 |
| ATOM | 637 | HD23 | LEU | 86 | −34.248 | −4.536 | −1.448 |
| ATOM | 638 | HD22 | LEU | 86 | −33.195 | −3.192 | −1.939 |
| ATOM | 639 | HD21 | LEU | 86 | −34.96 | −3.018 | −2.077 |
| ATOM | 640 | HA | LEU | 86 | −35.007 | −3.883 | 2.583 |
| ATOM | 641 | N | ALA | 87 | −38.117 | −4.686 | 1.747 |
| ATOM | 642 | CA | ALA | 87 | −39.446 | −4.651 | 2.343 |
| ATOM | 643 | C | ALA | 87 | −39.414 | −5.168 | 3.762 |
| ATOM | 644 | O | ALA | 87 | −40.043 | −4.573 | 4.624 |
| ATOM | 645 | CB | ALA | 87 | −40.469 | −5.454 | 1.502 |
| ATOM | 646 | H | ALA | 87 | −37.986 | −5.126 | 0.857 |
| ATOM | 647 | HB3 | ALA | 87 | −40.16 | −6.509 | 1.44 |
| ATOM | 648 | HB2 | ALA | 87 | −40.535 | −5.042 | 0.483 |
| ATOM | 649 | HB1 | ALA | 87 | −41.461 | −5.394 | 1.972 |
| ATOM | 650 | HA | ALA | 87 | −39.75 | −3.597 | 2.379 |
| ATOM | 651 | N | VAL | 88 | −38.673 | −6.263 | 4.042 |
| ATOM | 652 | CA | VAL | 88 | −38.544 | −6.694 | 5.433 |
| ATOM | 653 | C | VAL | 88 | −37.944 | −5.557 | 6.232 |
| ATOM | 654 | O | VAL | 88 | −38.558 | −5.119 | 7.193 |
| ATOM | 655 | CB | VAL | 88 | −37.709 | −8.003 | 5.593 |
| ATOM | 656 | CG1 | VAL | 88 | −37.317 | −8.261 | 7.075 |
| ATOM | 657 | CG2 | VAL | 88 | −38.488 | −9.228 | 5.04 |
| ATOM | 658 | H | VAL | 88 | −38.17 | −6.746 | 3.319 |
| ATOM | 659 | HB | VAL | 88 | −36.775 | −7.902 | 5.018 |
| ATOM | 660 | HG13 | VAL | 88 | −36.638 | −7.478 | 7.444 |
| ATOM | 661 | HG12 | VAL | 88 | −36.794 | −9.225 | 7.174 |
| ATOM | 662 | HG11 | VAL | 88 | −38.216 | −8.281 | 7.709 |
| ATOM | 663 | HG23 | VAL | 88 | −39.413 | −9.392 | 5.613 |
| ATOM | 664 | HG22 | VAL | 88 | −37.872 | −10.139 | 5.103 |
| ATOM | 665 | HG21 | VAL | 88 | −38.754 | −9.075 | 3.987 |
| ATOM | 666 | HA | VAL | 88 | −39.55 | −6.889 | 5.836 |
| ATOM | 667 | N | ALA | 89 | −36.742 | −5.064 | 5.86 |
| ATOM | 668 | CA | ALA | 89 | −36.092 | −4.045 | 6.686 |
| ATOM | 669 | C | ALA | 89 | −37.002 | −2.856 | 6.903 |
| ATOM | 670 | O | ALA | 89 | −37.153 | −2.394 | 8.023 |
| ATOM | 671 | CB | ALA | 89 | −34.775 | −3.527 | 6.051 |
| ATOM | 672 | H | ALA | 89 | −36.28 | −5.396 | 5.033 |
| ATOM | 673 | HB3 | ALA | 89 | −34.972 | −3.001 | 5.105 |
| ATOM | 674 | HB2 | ALA | 89 | −34.076 | −4.352 | 5.85 |
| ATOM | 675 | HB1 | ALA | 89 | −34.292 | −2.831 | 6.751 |
| ATOM | 676 | HA | ALA | 89 | −35.853 | −4.5 | 7.663 |
| ATOM | 677 | N | ASP | 90 | −37.617 | −2.338 | 5.821 |
| ATOM | 678 | CA | ASP | 90 | −38.45 | −1.149 | 5.947 |
| ATOM | 679 | C | ASP | 90 | −39.619 | −1.393 | 6.873 |
| ATOM | 680 | O | ASP | 90 | −39.984 | −0.489 | 7.611 |
| ATOM | 681 | CB | ASP | 90 | −38.935 | −0.665 | 4.559 |
| ATOM | 682 | CG | ASP | 90 | −37.751 | −0.248 | 3.721 |
| ATOM | 683 | OD1 | ASP | 90 | −36.814 | 0.397 | 4.273 |
| ATOM | 684 | OD2 | ASP | 90 | −37.747 | −0.552 | 2.5 |
| ATOM | 685 | H | ASP | 90 | −37.494 | −2.745 | 4.916 |
| ATOM | 686 | HB2 | ASP | 90 | −39.606 | 0.196 | 4.683 |
| ATOM | 687 | HB1 | ASP | 90 | −39.496 | −1.472 | 4.061 |
| ATOM | 688 | HA | ASP | 90 | −37.826 | −0.357 | 6.386 |
| ATOM | 689 | N | MET | 91 | −40.232 | −2.597 | 6.888 |
| ATOM | 690 | CA | MET | 91 | −41.268 | −2.836 | 7.893 |
| ATOM | 691 | C | MET | 91 | −40.644 | −2.752 | 9.27 |
| ATOM | 692 | O | MET | 91 | −41.261 | −2.169 | 10.15 |
| ATOM | 693 | CB | MET | 91 | −42.014 | −4.186 | 7.714 |
| ATOM | 694 | CG | MET | 91 | −42.911 | −4.158 | 6.449 |
| ATOM | 695 | SD | MET | 91 | −43.91 | −5.673 | 6.273 |
| ATOM | 696 | CE | MET | 91 | −42.665 | −6.903 | 5.773 |
| ATOM | 697 | HB2 | MET | 91 | −41.275 | −5 | 7.663 |
| ATOM | 698 | HB1 | MET | 91 | −42.661 | −4.36 | 8.59 |
| ATOM | 699 | HG2 | MET | 91 | −42.299 | −4.022 | 5.55 |
| ATOM | 700 | HG1 | MET | 91 | −43.626 | −3.322 | 6.51 |
| ATOM | 701 | HE3 | MET | 91 | −42.133 | −6.561 | 4.873 |
| ATOM | 702 | HE2 | MET | 91 | −43.176 | −7.852 | 5.556 |
| ATOM | 703 | HE1 | MET | 91 | −41.949 | −7.071 | 6.589 |
| ATOM | 704 | H | MET | 91 | −39.948 | −3.341 | 6.275 |
| ATOM | 705 | HA | MET | 91 | −42.017 | −2.03 | 7.819 |
| ATOM | 706 | LPD2 | MET | 91 | −44.388 | −5.599 | 5.796 |
| ATOM | 707 | LPD1 | MET | 91 | −44.203 | −5.844 | 6.861 |
| ATOM | 708 | N | LEU | 92 | −39.426 | −3.302 | 9.493 |
| ATOM | 709 | CA | LEU | 92 | −38.814 | −3.158 | 10.815 |
| ATOM | 710 | C | LEU | 92 | −38.649 | −1.686 | 11.123 |
| ATOM | 711 | O | LEU | 92 | −38.999 | −1.285 | 12.223 |
| ATOM | 712 | CB | LEU | 92 | −37.432 | −3.848 | 11.023 |
| ATOM | 713 | CG | LEU | 92 | −37.461 | −5.38 | 11.325 |
| ATOM | 714 | CD1 | LEU | 92 | −38.152 | −5.731 | 12.675 |
| ATOM | 715 | CD2 | LEU | 92 | −38.086 | −6.199 | 10.168 |
| ATOM | 716 | HB2 | LEU | 92 | −36.799 | −3.658 | 10.143 |
| ATOM | 717 | HB1 | LEU | 92 | −36.93 | −3.374 | 11.884 |
| ATOM | 718 | HG | LEU | 92 | −36.407 | −5.7 | 11.419 |
| ATOM | 719 | HD13 | LEU | 92 | −37.756 | −5.103 | 13.488 |
| ATOM | 720 | HD12 | LEU | 92 | −37.957 | −6.784 | 12.931 |
| ATOM | 721 | HD11 | LEU | 92 | −39.242 | −5.6 | 12.626 |
| ATOM | 722 | HD23 | LEU | 92 | −39.143 | −5.935 | 10.018 |
| ATOM | 723 | HD22 | LEU | 92 | −38.024 | −7.278 | 10.38 |
| ATOM | 724 | HD21 | LEU | 92 | −37.525 | −5.999 | 9.245 |
| ATOM | 725 | H | LEU | 92 | −38.919 | −3.766 | 8.762 |
| ATOM | 726 | HA | LEU | 92 | −39.514 | −3.564 | 11.556 |
| ATOM | 727 | N | VAL | 93 | −38.114 | −0.85 | 10.202 |
| ATOM | 728 | CA | VAL | 93 | −37.909 | 0.553 | 10.57 |
| ATOM | 729 | C | VAL | 93 | −39.249 | 1.2 | 10.828 |
| ATOM | 730 | O | VAL | 93 | −39.346 | 2.002 | 11.743 |
| ATOM | 731 | CB | VAL | 93 | −37.081 | 1.467 | 9.612 |
| ATOM | 732 | CG1 | VAL | 93 | −35.832 | 0.749 | 9.032 |
| ATOM | 733 | CG2 | VAL | 93 | −37.899 | 2.141 | 8.476 |
| ATOM | 734 | HB | VAL | 93 | −36.734 | 2.31 | 10.235 |
| ATOM | 735 | HG23 | VAL | 93 | −38.381 | 1.392 | 7.842 |
| ATOM | 736 | HG22 | VAL | 93 | −37.23 | 2.748 | 7.846 |
| ATOM | 737 | HG21 | VAL | 93 | −38.676 | 2.81 | 8.879 |
| ATOM | 738 | HG13 | VAL | 93 | −35.241 | 0.288 | 9.838 |
| ATOM | 739 | HG12 | VAL | 93 | −35.201 | 1.473 | 8.495 |
| ATOM | 740 | HG11 | VAL | 93 | −36.115 | −0.026 | 8.309 |
| ATOM | 741 | H | VAL | 93 | −37.875 | −1.18 | 9.287 |
| ATOM | 742 | HA | VAL | 93 | −37.351 | 0.53 | 11.524 |
| ATOM | 743 | N | SER | 94 | −40.291 | 0.884 | 10.028 |
| ATOM | 744 | CA | SER | 94 | −41.583 | 1.531 | 10.24 |
| ATOM | 745 | C | SER | 94 | −42.072 | 1.28 | 11.646 |
| ATOM | 746 | O | SER | 94 | −42.377 | 2.231 | 12.351 |
| ATOM | 747 | CB | SER | 94 | −42.663 | 1.027 | 9.248 |
| ATOM | 748 | OG | SER | 94 | −43.925 | 1.635 | 9.571 |
| ATOM | 749 | HB2 | SER | 94 | −42.356 | 1.295 | 8.225 |
| ATOM | 750 | HB1 | SER | 94 | −42.755 | −0.069 | 9.31 |
| ATOM | 751 | HG | SER | 94 | −44.624 | 1.382 | 8.973 |
| ATOM | 752 | H | SER | 94 | −40.193 | 0.225 | 9.277 |
| ATOM | 753 | HA | SER | 94 | −41.466 | 2.618 | 10.092 |
| ATOM | 754 | N | VAL | 95 | −42.16 | 0.003 | 12.08 |
| ATOM | 755 | CA | VAL | 95 | −42.695 | −0.26 | 13.415 |
| ATOM | 756 | C | VAL | 95 | −41.69 | 0.156 | 14.465 |
| ATOM | 757 | O | VAL | 95 | −42.096 | 0.674 | 15.492 |
| ATOM | 758 | CB | VAL | 95 | −43.164 | −1.73 | 13.648 |
| ATOM | 759 | CG1 | VAL | 95 | −44.211 | −2.156 | 12.58 |
| ATOM | 760 | CG2 | VAL | 95 | −41.993 | −2.75 | 13.703 |
| ATOM | 761 | HB | VAL | 95 | −43.661 | −1.764 | 14.635 |
| ATOM | 762 | HG23 | VAL | 95 | −41.441 | −2.757 | 12.756 |
| ATOM | 763 | HG22 | VAL | 95 | −42.381 | −3.764 | 13.881 |
| ATOM | 764 | HG21 | VAL | 95 | −41.292 | −2.511 | 14.518 |
| ATOM | 765 | HG13 | VAL | 95 | −45.047 | −1.44 | 12.552 |
| ATOM | 766 | HG12 | VAL | 95 | −44.619 | −3.15 | 12.821 |
| ATOM | 767 | HG11 | VAL | 95 | −43.762 | −2.205 | 11.576 |
| ATOM | 768 | H | VAL | 95 | −41.837 | −0.755 | 11.509 |
| ATOM | 769 | HA | VAL | 95 | −43.589 | 0.373 | 13.551 |
| ATOM | 770 | N | SER | 96 | −40.373 | −0.058 | 14.247 |
| ATOM | 771 | CA | SER | 96 | −39.407 | 0.282 | 15.29 |
| ATOM | 772 | C | SER | 96 | −39.38 | 1.784 | 15.473 |
| ATOM | 773 | O | SER | 96 | −39.432 | 2.25 | 16.601 |
| ATOM | 774 | CB | SER | 96 | −38.001 | −0.294 | 14.976 |
| ATOM | 775 | OG | SER | 96 | −37.168 | −0.212 | 16.146 |
| ATOM | 776 | HB2 | SER | 96 | −38.11 | −1.357 | 14.698 |
| ATOM | 777 | HB1 | SER | 96 | −37.55 | 0.251 | 14.13 |
| ATOM | 778 | H | SER | 96 | −40.043 | −0.446 | 13.383 |
| ATOM | 779 | HG | SER | 96 | −36.31 | −0.604 | 16.014 |
| ATOM | 780 | HA | SER | 96 | −39.736 | −0.185 | 16.233 |
| ATOM | 781 | N | ASN | 97 | −39.32 | 2.575 | 14.376 |
| ATOM | 782 | CA | ASN | 97 | −39.408 | 4.024 | 14.535 |
| ATOM | 783 | C | ASN | 97 | −40.755 | 4.367 | 15.121 |
| ATOM | 784 | O | ASN | 97 | −40.816 | 5.168 | 16.041 |
| ATOM | 785 | CB | ASN | 97 | −39.226 | 4.842 | 13.229 |
| ATOM | 786 | CG | ASN | 97 | −39.317 | 6.316 | 13.555 |
| ATOM | 787 | OD1 | ASN | 97 | −40.287 | 6.943 | 13.165 |
| ATOM | 788 | ND2 | ASN | 97 | −38.346 | 6.913 | 14.279 |
| ATOM | 789 | HB2 | ASN | 97 | −38.256 | 4.627 | 12.758 |
| ATOM | 790 | HB1 | ASN | 97 | −40.013 | 4.567 | 12.51 |
| ATOM | 791 | H | ASN | 97 | −39.268 | 2.191 | 13.454 |
| ATOM | 792 | HD21 | ASN | 97 | −37.532 | 6.427 | 14.606 |
| ATOM | 793 | HD22 | ASN | 97 | −38.441 | 7.882 | 14.51 |
| ATOM | 794 | HA | ASN | 97 | −38.627 | 4.321 | 15.244 |
| ATOM | 795 | N | GLY | 98 | −41.85 | 3.763 | 14.606 |
| ATOM | 796 | CA | GLY | 98 | −43.159 | 4.014 | 15.199 |
| ATOM | 797 | C | GLY | 98 | −43.072 | 3.881 | 16.7 |
| ATOM | 798 | O | GLY | 98 | −43.556 | 4.746 | 17.414 |
| ATOM | 799 | H | GLY | 98 | −41.79 | 3.131 | 13.827 |
| ATOM | 800 | HA1 | GLY | 98 | −43.506 | 5.017 | 14.91 |
| ATOM | 801 | HA2 | GLY | 98 | −43.899 | 3.28 | 14.843 |
| ATOM | 802 | N | SER | 99 | −42.445 | 2.787 | 17.187 |
| ATOM | 803 | CA | SER | 99 | −42.331 | 2.585 | 18.627 |
| ATOM | 804 | C | SER | 99 | −41.489 | 3.659 | 19.275 |
| ATOM | 805 | O | SER | 99 | −41.962 | 4.246 | 20.236 |
| ATOM | 806 | CB | SER | 99 | −41.737 | 1.194 | 18.968 |
| ATOM | 807 | OG | SER | 99 | −42.585 | 0.178 | 18.402 |
| ATOM | 808 | HB2 | SER | 99 | −40.718 | 1.111 | 18.555 |
| ATOM | 809 | HB1 | SER | 99 | −41.684 | 1.074 | 20.064 |
| ATOM | 810 | HG | SER | 99 | −42.266 | −0.705 | 18.56 |
| ATOM | 811 | H | SER | 99 | −42.06 | 2.092 | 16.574 |
| ATOM | 812 | HA | SER | 99 | −43.347 | 2.612 | 19.056 |
| ATOM | 813 | N | GLU | 100 | −40.247 | 3.945 | 18.809 |
| ATOM | 814 | CA | GLU | 100 | −39.453 | 4.944 | 19.528 |
| ATOM | 815 | C | GLU | 100 | −40.239 | 6.225 | 19.573 |
| ATOM | 816 | O | GLU | 100 | −40.312 | 6.852 | 20.617 |
| ATOM | 817 | CB | GLU | 100 | −38.023 | 5.267 | 18.981 |
| ATOM | 818 | CG | GLU | 100 | −37.982 | 6.264 | 17.783 |
| ATOM | 819 | CD | GLU | 100 | −36.606 | 6.588 | 17.259 |
| ATOM | 820 | OE1 | GLU | 100 | −36.338 | 6.395 | 16.039 |
| ATOM | 821 | OE2 | GLU | 100 | −35.763 | 7.076 | 18.056 |
| ATOM | 822 | HB2 | GLU | 100 | −37.435 | 5.735 | 19.789 |
| ATOM | 823 | HB1 | GLU | 100 | −37.535 | 4.315 | 18.729 |
| ATOM | 824 | HG2 | GLU | 100 | −38.392 | 7.242 | 18.076 |
| ATOM | 825 | HG1 | GLU | 100 | −38.586 | 5.87 | 16.961 |
| ATOM | 826 | H | GLU | 100 | −39.863 | 3.491 | 18 |
| ATOM | 827 | HA | GLU | 100 | −39.331 | 4.565 | 20.558 |
| ATOM | 828 | N | THR | 101 | −40.82 | 6.652 | 18.432 |
| ATOM | 829 | CA | THR | 101 | −41.318 | 8.016 | 18.383 |
| ATOM | 830 | C | THR | 101 | −42.566 | 8.121 | 19.218 |
| ATOM | 831 | O | THR | 101 | −42.621 | 9.022 | 20.036 |
| ATOM | 832 | CB | THR | 101 | −41.447 | 8.577 | 16.94 |
| ATOM | 833 | OG1 | THR | 101 | −41.47 | 10.013 | 17.031 |
| ATOM | 834 | CG2 | THR | 101 | −42.709 | 8.073 | 16.196 |
| ATOM | 835 | H | THR | 101 | −40.875 | 6.081 | 17.609 |
| ATOM | 836 | HB | THR | 101 | −40.544 | 8.269 | 16.381 |
| ATOM | 837 | HG1 | THR | 101 | −41.45 | 10.443 | 16.184 |
| ATOM | 838 | HG22 | THR | 101 | −43.621 | 8.457 | 16.676 |
| ATOM | 839 | HG21 | THR | 101 | −42.739 | 6.977 | 16.183 |
| ATOM | 840 | HG23 | THR | 101 | −42.689 | 8.422 | 15.155 |
| ATOM | 841 | HA | THR | 101 | −40.55 | 8.658 | 18.851 |
| ATOM | 842 | N | ILE | 102 | −43.584 | 7.242 | 19.076 |
| ATOM | 843 | CA | ILE | 102 | −44.815 | 7.47 | 19.839 |
| ATOM | 844 | C | ILE | 102 | −44.564 | 7.353 | 21.33 |
| ATOM | 845 | O | ILE | 102 | −45.221 | 8.054 | 22.083 |
| ATOM | 846 | CB | ILE | 102 | −46.018 | 6.623 | 19.325 |
| ATOM | 847 | CG1 | ILE | 102 | −47.404 | 7.156 | 19.811 |
| ATOM | 848 | CG2 | ILE | 102 | −45.879 | 5.13 | 19.731 |
| ATOM | 849 | CD1 | ILE | 102 | −47.766 | 8.587 | 19.322 |
| ATOM | 850 | HB | ILE | 102 | −46.004 | 6.68 | 18.222 |
| ATOM | 851 | HG23 | ILE | 102 | −44.861 | 4.75 | 19.57 |
| ATOM | 852 | HG22 | ILE | 102 | −46.576 | 4.507 | 19.149 |
| ATOM | 853 | HG21 | ILE | 102 | −46.127 | 5.013 | 20.792 |
| ATOM | 854 | HG12 | ILE | 102 | −47.455 | 7.134 | 20.912 |
| ATOM | 855 | HG11 | ILE | 102 | −48.181 | 6.473 | 19.425 |
| ATOM | 856 | HD13 | ILE | 102 | −47.19 | 9.363 | 19.848 |
| ATOM | 857 | HD12 | ILE | 102 | −48.832 | 8.786 | 19.519 |
| ATOM | 858 | HD11 | ILE | 102 | −47.591 | 8.682 | 18.239 |
| ATOM | 859 | H | ILE | 102 | −43.512 | 6.452 | 18.463 |
| ATOM | 860 | HA | ILE | 102 | −45.07 | 8.52 | 19.648 |
| ATOM | 861 | N | ILE | 103 | −43.613 | 6.506 | 21.792 |
| ATOM | 862 | CA | ILE | 103 | −43.261 | 6.509 | 23.218 |
| ATOM | 863 | C | ILE | 103 | −42.531 | 7.801 | 23.532 |
| ATOM | 864 | O | ILE | 103 | −42.927 | 8.497 | 24.454 |
| ATOM | 865 | CB | ILE | 103 | −42.406 | 5.264 | 23.637 |
| ATOM | 866 | CG1 | ILE | 103 | −43.278 | 4.005 | 23.938 |
| ATOM | 867 | CG2 | ILE | 103 | −41.533 | 5.546 | 24.896 |
| ATOM | 868 | CD1 | ILE | 103 | −44.298 | 3.652 | 22.828 |
| ATOM | 869 | HB | ILE | 103 | −41.701 | 5.025 | 22.822 |
| ATOM | 870 | HG23 | ILE | 103 | −40.79 | 6.331 | 24.702 |
| ATOM | 871 | HG22 | ILE | 103 | −40.981 | 4.642 | 25.197 |
| ATOM | 872 | HG21 | ILE | 103 | −42.169 | 5.86 | 25.737 |
| ATOM | 873 | HG12 | ILE | 103 | −43.838 | 4.159 | 24.876 |
| ATOM | 874 | HG11 | ILE | 103 | −42.614 | 3.135 | 24.081 |
| ATOM | 875 | HD13 | ILE | 103 | −45.065 | 4.435 | 22.773 |
| ATOM | 876 | HD12 | ILE | 103 | −44.801 | 2.701 | 23.064 |
| ATOM | 877 | HD11 | ILE | 103 | −43.803 | 3.55 | 21.852 |
| ATOM | 878 | H | ILE | 103 | −43.088 | 5.934 | 21.157 |
| ATOM | 879 | HA | ILE | 103 | −44.177 | 6.507 | 23.832 |
| ATOM | 880 | N | ILE | 104 | −41.447 | 8.137 | 22.798 |
| ATOM | 881 | CA | ILE | 104 | −40.656 | 9.323 | 23.146 |
| ATOM | 882 | C | ILE | 104 | −41.452 | 10.603 | 22.968 |
| ATOM | 883 | O | ILE | 104 | −41.164 | 11.581 | 23.641 |
| ATOM | 884 | CB | ILE | 104 | −39.288 | 9.337 | 22.395 |
| ATOM | 885 | CG1 | ILE | 104 | −38.375 | 8.124 | 22.785 |
| ATOM | 886 | CG2 | ILE | 104 | −38.541 | 10.69 | 22.577 |
| ATOM | 887 | CD1 | ILE | 104 | −37.96 | 8.047 | 24.281 |
| ATOM | 888 | HB | ILE | 104 | −39.508 | 9.256 | 21.316 |
| ATOM | 889 | HG23 | ILE | 104 | −39.083 | 11.507 | 22.075 |
| ATOM | 890 | HG22 | ILE | 104 | −37.534 | 10.644 | 22.134 |
| ATOM | 891 | HG21 | ILE | 104 | −38.445 | 10.95 | 23.639 |
| ATOM | 892 | HG12 | ILE | 104 | −37.465 | 8.172 | 22.165 |
| ATOM | 893 | HG11 | ILE | 104 | −38.864 | 7.167 | 22.552 |
| ATOM | 894 | HD13 | ILE | 104 | −37.589 | 9.009 | 24.644 |
| ATOM | 895 | HD12 | ILE | 104 | −37.154 | 7.308 | 24.413 |
| ATOM | 896 | HD11 | ILE | 104 | −38.797 | 7.741 | 24.923 |
| ATOM | 897 | H | ILE | 104 | −41.182 | 7.582 | 22.006 |
| ATOM | 898 | HA | ILE | 104 | −40.425 | 9.282 | 24.217 |
| ATOM | 899 | N | THR | 105 | −42.473 | 10.633 | 22.085 |
| ATOM | 900 | CA | THR | 105 | −43.289 | 11.836 | 21.913 |
| ATOM | 901 | C | THR | 105 | −43.886 | 12.35 | 23.203 |
| ATOM | 902 | O | THR | 105 | −44.182 | 13.534 | 23.26 |
| ATOM | 903 | CB | THR | 105 | −44.466 | 11.536 | 20.938 |
| ATOM | 904 | OG1 | THR | 105 | −43.891 | 11.196 | 19.667 |
| ATOM | 905 | CG2 | THR | 105 | −45.428 | 12.738 | 20.738 |
| ATOM | 906 | HB | THR | 105 | −45.038 | 10.68 | 21.34 |
| ATOM | 907 | HG22 | THR | 105 | −46.145 | 12.525 | 19.931 |
| ATOM | 908 | HG21 | THR | 105 | −44.847 | 13.632 | 20.465 |
| ATOM | 909 | HG23 | THR | 105 | −46.007 | 12.944 | 21.65 |
| ATOM | 910 | HG1 | THR | 105 | −44.533 | 10.882 | 19.041 |
| ATOM | 911 | H | THR | 105 | −42.68 | 9.835 | 21.518 |
| ATOM | 912 | HA | THR | 105 | −42.658 | 12.625 | 21.474 |
| ATOM | 913 | N | LEU | 106 | −44.103 | 11.528 | 24.253 |
| ATOM | 914 | CA | LEU | 106 | −44.909 | 11.99 | 25.386 |
| ATOM | 915 | C | LEU | 106 | −44.136 | 12.891 | 26.33 |
| ATOM | 916 | O | LEU | 106 | −44.145 | 12.641 | 27.525 |
| ATOM | 917 | CB | LEU | 106 | −45.563 | 10.781 | 26.121 |
| ATOM | 918 | CG | LEU | 106 | −46.321 | 9.778 | 25.193 |
| ATOM | 919 | CD1 | LEU | 106 | −47.005 | 8.679 | 26.054 |
| ATOM | 920 | CD2 | LEU | 106 | −47.37 | 10.478 | 24.286 |
| ATOM | 921 | HB2 | LEU | 106 | −44.768 | 10.227 | 26.647 |
| ATOM | 922 | HB1 | LEU | 106 | −46.263 | 11.169 | 26.879 |
| ATOM | 923 | HG | LEU | 106 | −45.607 | 9.257 | 24.533 |
| ATOM | 924 | HD13 | LEU | 106 | −46.261 | 8.163 | 26.681 |
| ATOM | 925 | HD12 | LEU | 106 | −47.491 | 7.928 | 25.412 |
| ATOM | 926 | HD11 | LEU | 106 | −47.769 | 9.124 | 26.71 |
| ATOM | 927 | HD23 | LEU | 106 | −48.065 | 11.076 | 24.896 |
| ATOM | 928 | HD22 | LEU | 106 | −47.951 | 9.73 | 23.724 |
| ATOM | 929 | HD21 | LEU | 106 | −46.882 | 11.135 | 23.551 |
| ATOM | 930 | H | LEU | 106 | −43.728 | 10.599 | 24.274 |
| ATOM | 931 | HA | LEU | 106 | −45.734 | 12.623 | 25.021 |
| ATOM | 932 | N | LEU | 107 | −43.47 | 13.96 | 25.831 |
| ATOM | 933 | CA | LEU | 107 | −42.716 | 14.85 | 26.712 |
| ATOM | 934 | C | LEU | 107 | −41.766 | 14.029 | 27.558 |
| ATOM | 935 | O | LEU | 107 | −41.629 | 14.28 | 28.745 |
| ATOM | 936 | CB | LEU | 107 | −43.643 | 15.748 | 27.582 |
| ATOM | 937 | CG | LEU | 107 | −44.645 | 16.666 | 26.812 |
| ATOM | 938 | CD1 | LEU | 107 | −43.938 | 17.587 | 25.779 |
| ATOM | 939 | CD2 | LEU | 107 | −45.821 | 15.896 | 26.147 |
| ATOM | 940 | HB2 | LEU | 107 | −44.21 | 15.109 | 28.277 |
| ATOM | 941 | HB1 | LEU | 107 | −42.994 | 16.399 | 28.194 |
| ATOM | 942 | HG | LEU | 107 | −45.101 | 17.322 | 27.577 |
| ATOM | 943 | HD13 | LEU | 107 | −43.093 | 18.114 | 26.247 |
| ATOM | 944 | HD12 | LEU | 107 | −44.644 | 18.34 | 25.396 |
| ATOM | 945 | HD11 | LEU | 107 | −43.564 | 17.004 | 24.924 |
| ATOM | 946 | HD23 | LEU | 107 | −46.604 | 16.606 | 25.836 |
| ATOM | 947 | HD22 | LEU | 107 | −45.502 | 15.349 | 25.251 |
| ATOM | 948 | HD21 | LEU | 107 | −46.269 | 15.181 | 26.855 |
| ATOM | 949 | H | LEU | 107 | −43.476 | 14.168 | 24.851 |
| ATOM | 950 | HA | LEU | 107 | −42.056 | 15.503 | 26.117 |
| ATOM | 951 | N | ASN | 108 | −41.094 | 13.021 | 26.958 |
| ATOM | 952 | CA | ASN | 108 | −40.25 | 12.131 | 27.753 |
| ATOM | 953 | C | ASN | 108 | −38.991 | 12.866 | 28.163 |
| ATOM | 954 | O | ASN | 108 | −37.937 | 12.615 | 27.602 |
| ATOM | 955 | CB | ASN | 108 | −39.947 | 10.812 | 26.99 |
| ATOM | 956 | CG | ASN | 108 | −41.23 | 10.117 | 26.598 |
| ATOM | 957 | OD1 | ASN | 108 | −42.183 | 10.794 | 26.253 |
| ATOM | 958 | ND2 | ASN | 108 | −41.309 | 8.771 | 26.614 |
| ATOM | 959 | H2 | ASN | 108 | −41.191 | 12.841 | 25.975 |
| ATOM | 960 | HA | ASN | 108 | −40.794 | 11.835 | 28.667 |
| ATOM | 961 | HB2 | ASN | 108 | −39.378 | 11.029 | 26.074 |
| ATOM | 962 | HB1 | ASN | 108 | −39.341 | 10.153 | 27.628 |
| ATOM | 963 | HD21 | ASN | 108 | −40.543 | 8.191 | 26.888 |
| ATOM | 964 | HD22 | ASN | 108 | −42.169 | 8.349 | 26.32 |
| ATOM | 965 | N | SER | 109 | −39.072 | 13.784 | 29.153 |
| ATOM | 966 | CA | SER | 109 | −37.874 | 14.492 | 29.608 |
| ATOM | 967 | C | SER | 109 | −36.989 | 13.528 | 30.371 |
| ATOM | 968 | O | SER | 109 | −36.956 | 13.567 | 31.591 |
| ATOM | 969 | CB | SER | 109 | −38.251 | 15.723 | 30.475 |
| ATOM | 970 | OG | SER | 109 | −37.054 | 16.445 | 30.818 |
| ATOM | 971 | H | SER | 109 | −39.951 | 13.974 | 29.599 |
| ATOM | 972 | HA | SER | 109 | −37.314 | 14.88 | 28.74 |
| ATOM | 973 | HB2 | SER | 109 | −38.927 | 16.375 | 29.895 |
| ATOM | 974 | HB1 | SER | 109 | −38.778 | 15.39 | 31.385 |
| ATOM | 975 | HG | SER | 109 | −37.223 | 17.211 | 31.358 |
| ATOM | 976 | N | THR | 110 | −36.259 | 12.64 | 29.661 |
| ATOM | 977 | CA | THR | 110 | −35.367 | 11.694 | 30.333 |
| ATOM | 978 | C | THR | 110 | −34.048 | 12.371 | 30.639 |
| ATOM | 979 | O | THR | 110 | −33.576 | 12.278 | 31.761 |
| ATOM | 980 | CB | THR | 110 | −35.109 | 10.476 | 29.406 |
| ATOM | 981 | OG1 | THR | 110 | −34.517 | 10.981 | 28.199 |
| ATOM | 982 | CG2 | THR | 110 | −36.416 | 9.699 | 29.097 |
| ATOM | 983 | H | THR | 110 | −36.294 | 12.636 | 28.659 |
| ATOM | 984 | HA | THR | 110 | −35.82 | 11.33 | 31.27 |
| ATOM | 985 | HB | THR | 110 | −34.413 | 9.785 | 29.903 |
| ATOM | 986 | HG22 | THR | 110 | −36.209 | 8.844 | 28.436 |
| ATOM | 987 | HG21 | THR | 110 | −37.136 | 10.352 | 28.591 |
| ATOM | 988 | HG23 | THR | 110 | −36.871 | 9.322 | 30.026 |
| ATOM | 989 | HG1 | THR | 110 | −34.276 | 10.291 | 27.593 |
| ATOM | 990 | N | ASP | 111 | −33.437 | 13.054 | 29.643 |
| ATOM | 991 | CA | ASP | 111 | −32.171 | 13.758 | 29.862 |
| ATOM | 992 | C | ASP | 111 | −32.188 | 15.035 | 29.045 |
| ATOM | 993 | O | ASP | 111 | −33.214 | 15.32 | 28.448 |
| ATOM | 994 | CB | ASP | 111 | −31.042 | 12.786 | 29.42 |
| ATOM | 995 | CG | ASP | 111 | −29.659 | 13.358 | 29.572 |
| ATOM | 996 | OD1 | ASP | 111 | −29.488 | 14.272 | 30.424 |
| ATOM | 997 | OD2 | ASP | 111 | −28.736 | 12.932 | 28.832 |
| ATOM | 998 | H | ASP | 111 | −33.813 | 13.069 | 28.712 |
| ATOM | 999 | HA | ASP | 111 | −32.038 | 14.045 | 30.918 |
| ATOM | 1000 | HB2 | ASP | 111 | −31.096 | 11.881 | 30.038 |
| ATOM | 1001 | HB1 | ASP | 111 | −31.194 | 12.492 | 28.37 |
| ATOM | 1002 | N | THR | 112 | −31.091 | 15.825 | 28.978 |
| ATOM | 1003 | CA | THR | 112 | −31.031 | 16.909 | 27.997 |
| ATOM | 1004 | C | THR | 112 | −31.545 | 16.431 | 26.654 |
| ATOM | 1005 | O | THR | 112 | −32.247 | 17.19 | 26.003 |
| ATOM | 1006 | CB | THR | 112 | −29.606 | 17.516 | 27.854 |
| ATOM | 1007 | OG1 | THR | 112 | −29.694 | 18.682 | 27.014 |
| ATOM | 1008 | CG2 | THR | 112 | −28.576 | 16.514 | 27.263 |
| ATOM | 1009 | H | THR | 112 | −30.311 | 15.679 | 29.59 |
| ATOM | 1010 | HA | THR | 112 | −31.7 | 17.71 | 28.355 |
| ATOM | 1011 | HB | THR | 112 | −29.269 | 17.825 | 28.861 |
| ATOM | 1012 | HG22 | THR | 112 | −27.57 | 16.961 | 27.277 |
| ATOM | 1013 | HG21 | THR | 112 | −28.827 | 16.275 | 26.219 |
| ATOM | 1014 | HG23 | THR | 112 | −28.544 | 15.583 | 27.848 |
| ATOM | 1015 | HG1 | THR | 112 | −28.861 | 19.132 | 26.91 |
| ATOM | 1016 | N | ASP | 113 | −31.239 | 15.177 | 26.234 |
| ATOM | 1017 | CA | ASP | 113 | −31.899 | 14.601 | 25.061 |
| ATOM | 1018 | C | ASP | 113 | −33.052 | 13.766 | 25.574 |
| ATOM | 1019 | O | ASP | 113 | −32.995 | 13.302 | 26.702 |
| ATOM | 1020 | CB | ASP | 113 | −30.934 | 13.744 | 24.2 |
| ATOM | 1021 | CG | ASP | 113 | −31.494 | 13.432 | 22.834 |
| ATOM | 1022 | OD1 | ASP | 113 | −32.735 | 13.554 | 22.636 |
| ATOM | 1023 | OD2 | ASP | 113 | −30.687 | 13.068 | 21.936 |
| ATOM | 1024 | H | ASP | 113 | −30.658 | 14.567 | 26.777 |
| ATOM | 1025 | HA | ASP | 113 | −32.287 | 15.4 | 24.407 |
| ATOM | 1026 | HB2 | ASP | 113 | −30.003 | 14.314 | 24.059 |
| ATOM | 1027 | HB1 | ASP | 113 | −30.69 | 12.806 | 24.722 |
| ATOM | 1028 | N | ALA | 114 | −34.124 | 13.55 | 24.783 |
| ATOM | 1029 | CA | ALA | 114 | −35.301 | 12.874 | 25.324 |
| ATOM | 1030 | C | ALA | 114 | −35.183 | 11.366 | 25.309 |
| ATOM | 1031 | O | ALA | 114 | −36.186 | 10.731 | 25.59 |
| ATOM | 1032 | CB | ALA | 114 | −36.563 | 13.317 | 24.536 |
| ATOM | 1033 | H | ALA | 114 | −34.104 | 13.829 | 23.817 |
| ATOM | 1034 | HA | ALA | 114 | −35.456 | 13.18 | 26.371 |
| ATOM | 1035 | HB3 | ALA | 114 | −36.497 | 12.967 | 23.494 |
| ATOM | 1036 | HB2 | ALA | 114 | −36.636 | 14.415 | 24.537 |
| ATOM | 1037 | HB1 | ALA | 114 | −37.48 | 12.908 | 24.988 |
| ATOM | 1038 | N | GLN | 115 | −34.021 | 10.747 | 24.991 |
| ATOM | 1039 | CA | GLN | 115 | −34.004 | 9.3 | 24.768 |
| ATOM | 1040 | C | GLN | 115 | −32.69 | 8.593 | 25.057 |
| ATOM | 1041 | O | GLN | 115 | −32.482 | 7.527 | 24.497 |
| ATOM | 1042 | CB | GLN | 115 | −34.457 | 9.105 | 23.291 |
| ATOM | 1043 | CG | GLN | 115 | −33.773 | 10.07 | 22.272 |
| ATOM | 1044 | CD | GLN | 115 | −32.287 | 10.296 | 22.442 |
| ATOM | 1045 | OE1 | GLN | 115 | −31.856 | 10.582 | 23.548 |
| ATOM | 1046 | NE2 | GLN | 115 | −31.461 | 10.222 | 21.379 |
| ATOM | 1047 | H | GLN | 115 | −33.188 | 11.26 | 24.773 |
| ATOM | 1048 | HA | GLN | 115 | −34.719 | 8.785 | 25.43 |
| ATOM | 1049 | HB2 | GLN | 115 | −35.535 | 9.317 | 23.238 |
| ATOM | 1050 | HB1 | GLN | 115 | −34.329 | 8.055 | 22.989 |
| ATOM | 1051 | HG2 | GLN | 115 | −34.007 | 9.716 | 21.255 |
| ATOM | 1052 | HG1 | GLN | 115 | −34.218 | 11.073 | 22.373 |
| ATOM | 1053 | HE21 | GLN | 115 | −30.494 | 10.442 | 21.502 |
| ATOM | 1054 | HE22 | GLN | 115 | −31.79 | 9.98 | 20.464 |
| ATOM | 1055 | N | SER | 116 | −31.789 | 9.125 | 25.918 |
| ATOM | 1056 | CA | SER | 116 | −30.476 | 8.497 | 26.086 |
| ATOM | 1057 | C | SER | 116 | −29.873 | 8.784 | 27.447 |
| ATOM | 1058 | O | SER | 116 | −30.367 | 9.661 | 28.138 |
| ATOM | 1059 | CB | SER | 116 | −29.517 | 9 | 24.977 |
| ATOM | 1060 | OG | SER | 116 | −29.894 | 8.429 | 23.712 |
| ATOM | 1061 | H | SER | 116 | −31.954 | 9.984 | 26.405 |
| ATOM | 1062 | HA | SER | 116 | −30.559 | 7.406 | 25.983 |
| ATOM | 1063 | HB2 | SER | 116 | −29.564 | 10.101 | 24.939 |
| ATOM | 1064 | HB1 | SER | 116 | −28.481 | 8.695 | 25.199 |
| ATOM | 1065 | HG | SER | 116 | −29.34 | 8.725 | 22.997 |
| ATOM | 1066 | N | PHE | 117 | −28.804 | 8.045 | 27.836 |
| ATOM | 1067 | CA | PHE | 117 | −28.105 | 8.303 | 29.097 |
| ATOM | 1068 | C | PHE | 117 | −28.971 | 7.991 | 30.302 |
| ATOM | 1069 | O | PHE | 117 | −28.699 | 7.011 | 30.979 |
| ATOM | 1070 | CB | PHE | 117 | −27.476 | 9.723 | 29.11 |
| ATOM | 1071 | CG | PHE | 117 | −26.754 | 9.951 | 27.773 |
| ATOM | 1072 | CD1 | PHE | 117 | −27.324 | 10.755 | 26.778 |
| ATOM | 1073 | CD2 | PHE | 117 | −25.517 | 9.342 | 27.535 |
| ATOM | 1074 | CE1 | PHE | 117 | −26.678 | 10.926 | 25.55 |
| ATOM | 1075 | CE2 | PHE | 117 | −24.874 | 9.503 | 26.304 |
| ATOM | 1076 | CZ | PHE | 117 | −25.457 | 10.291 | 25.308 |
| ATOM | 1077 | H | PHE | 117 | −28.472 | 7.287 | 27.267 |
| ATOM | 1078 | HA | PHE | 117 | −27.247 | 7.608 | 29.156 |
| ATOM | 1079 | HB2 | PHE | 117 | −28.241 | 10.497 | 29.252 |
| ATOM | 1080 | HB1 | PHE | 117 | −26.763 | 9.819 | 29.944 |
| ATOM | 1081 | HD1 | PHE | 117 | −28.274 | 11.249 | 26.949 |
| ATOM | 1082 | HD2 | PHE | 117 | −25.05 | 8.737 | 28.306 |
| ATOM | 1083 | HE1 | PHE | 117 | −27.127 | 11.55 | 24.784 |
| ATOM | 1084 | HE2 | PHE | 117 | −23.921 | 9.016 | 26.121 |
| ATOM | 1085 | HZ | PHE | 117 | −24.962 | 10.411 | 24.348 |
| ATOM | 1086 | N | THR | 118 | −30.025 | 8.782 | 30.601 |
| ATOM | 1087 | CA | THR | 118 | −30.92 | 8.422 | 31.704 |
| ATOM | 1088 | C | THR | 118 | −31.613 | 7.136 | 31.308 |
| ATOM | 1089 | O | THR | 118 | −31.634 | 6.203 | 32.095 |
| ATOM | 1090 | CB | THR | 118 | −31.938 | 9.556 | 32.003 |
| ATOM | 1091 | OG1 | THR | 118 | −31.216 | 10.704 | 32.484 |
| ATOM | 1092 | CG2 | THR | 118 | −33.005 | 9.12 | 33.042 |
| ATOM | 1093 | H | THR | 118 | −30.248 | 9.57 | 30.027 |
| ATOM | 1094 | HA | THR | 118 | −30.327 | 8.254 | 32.618 |
| ATOM | 1095 | HB | THR | 118 | −32.435 | 9.823 | 31.06 |
| ATOM | 1096 | HG22 | THR | 118 | −33.7 | 9.95 | 33.245 |
| ATOM | 1097 | HG21 | THR | 118 | −32.515 | 8.834 | 33.984 |
| ATOM | 1098 | HG23 | THR | 118 | −33.593 | 8.266 | 32.672 |
| ATOM | 1099 | HG1 | THR | 118 | −31.778 | 11.465 | 32.613 |
| ATOM | 1100 | N | VAL | 119 | −32.161 | 7.073 | 30.073 |
| ATOM | 1101 | CA | VAL | 119 | −32.589 | 5.796 | 29.495 |
| ATOM | 1102 | C | VAL | 119 | −31.559 | 5.473 | 28.44 |
| ATOM | 1103 | O | VAL | 119 | −30.738 | 6.333 | 28.18 |
| ATOM | 1104 | CB | VAL | 119 | −34.025 | 5.858 | 28.893 |
| ATOM | 1105 | CG1 | VAL | 119 | −35.031 | 6.326 | 29.979 |
| ATOM | 1106 | CG2 | VAL | 119 | −34.115 | 6.759 | 27.629 |
| ATOM | 1107 | H | VAL | 119 | −32.138 | 7.868 | 29.466 |
| ATOM | 1108 | HA | VAL | 119 | −32.563 | 4.984 | 30.239 |
| ATOM | 1109 | HB | VAL | 119 | −34.314 | 4.838 | 28.581 |
| ATOM | 1110 | HG23 | VAL | 119 | −33.871 | 7.803 | 27.865 |
| ATOM | 1111 | HG22 | VAL | 119 | −35.134 | 6.728 | 27.214 |
| ATOM | 1112 | HG21 | VAL | 119 | −33.425 | 6.41 | 26.847 |
| ATOM | 1113 | HG13 | VAL | 119 | −34.999 | 5.649 | 30.847 |
| ATOM | 1114 | HG12 | VAL | 119 | −36.057 | 6.331 | 29.58 |
| ATOM | 1115 | HG11 | VAL | 119 | −34.783 | 7.34 | 30.323 |
| ATOM | 1116 | N | ASN | 120 | −31.541 | 4.283 | 27.804 |
| ATOM | 1117 | CA | ASN | 120 | −30.526 | 4.03 | 26.778 |
| ATOM | 1118 | C | ASN | 120 | −31.129 | 3.176 | 25.69 |
| ATOM | 1119 | O | ASN | 120 | −30.686 | 2.055 | 25.496 |
| ATOM | 1120 | CB | ASN | 120 | −29.251 | 3.429 | 27.42 |
| ATOM | 1121 | CG | ASN | 120 | −28.626 | 4.459 | 28.334 |
| ATOM | 1122 | OD1 | ASN | 120 | −27.782 | 5.212 | 27.874 |
| ATOM | 1123 | ND2 | ASN | 120 | −29.023 | 4.542 | 29.621 |
| ATOM | 1124 | H | ASN | 120 | −32.184 | 3.553 | 28.039 |
| ATOM | 1125 | HA | ASN | 120 | −30.222 | 4.954 | 26.259 |
| ATOM | 1126 | HB2 | ASN | 120 | −29.508 | 2.514 | 27.976 |
| ATOM | 1127 | HB1 | ASN | 120 | −28.527 | 3.16 | 26.634 |
| ATOM | 1128 | HD21 | ASN | 120 | −29.717 | 3.932 | 30.001 |
| ATOM | 1129 | HD22 | ASN | 120 | −28.625 | 5.249 | 30.211 |
| ATOM | 1130 | N | ILE | 121 | −32.149 | 3.72 | 24.983 |
| ATOM | 1131 | CA | ILE | 121 | −32.79 | 2.991 | 23.886 |
| ATOM | 1132 | C | ILE | 121 | −32.498 | 3.576 | 22.52 |
| ATOM | 1133 | O | ILE | 121 | −32.731 | 2.862 | 21.556 |
| ATOM | 1134 | CB | ILE | 121 | −34.33 | 2.879 | 24.117 |
| ATOM | 1135 | CG1 | ILE | 121 | −35.085 | 4.22 | 23.851 |
| ATOM | 1136 | CG2 | ILE | 121 | −34.601 | 2.329 | 25.546 |
| ATOM | 1137 | CD1 | ILE | 121 | −36.63 | 4.07 | 23.924 |
| ATOM | 1138 | H | ILE | 121 | −32.487 | 4.634 | 25.209 |
| ATOM | 1139 | HA | ILE | 121 | −32.401 | 1.959 | 23.841 |
| ATOM | 1140 | HB | ILE | 121 | −34.714 | 2.141 | 23.39 |
| ATOM | 1141 | HG23 | ILE | 121 | −33.984 | 1.437 | 25.737 |
| ATOM | 1142 | HG22 | ILE | 121 | −35.654 | 2.035 | 25.668 |
| ATOM | 1143 | HG21 | ILE | 121 | −34.358 | 3.088 | 26.305 |
| ATOM | 1144 | HG12 | ILE | 121 | −34.846 | 4.579 | 22.836 |
| ATOM | 1145 | HG11 | ILE | 121 | −34.771 | 4.997 | 24.565 |
| ATOM | 1146 | HD13 | ILE | 121 | −36.968 | 3.23 | 23.298 |
| ATOM | 1147 | HD12 | ILE | 121 | −37.118 | 4.985 | 23.556 |
| ATOM | 1148 | HD11 | ILE | 121 | −36.966 | 3.902 | 24.958 |
| ATOM | 1149 | N | ASP | 122 | −32.014 | 4.834 | 22.361 |
| ATOM | 1150 | CA | ASP | 122 | −31.797 | 5.358 | 21.011 |
| ATOM | 1151 | C | ASP | 122 | −30.553 | 4.711 | 20.441 |
| ATOM | 1152 | O | ASP | 122 | −29.515 | 5.346 | 20.342 |
| ATOM | 1153 | CB | ASP | 122 | −31.709 | 6.905 | 20.983 |
| ATOM | 1154 | CG | ASP | 122 | −31.841 | 7.421 | 19.575 |
| ATOM | 1155 | OD1 | ASP | 122 | −32.206 | 8.62 | 19.412 |
| ATOM | 1156 | OD2 | ASP | 122 | −31.601 | 6.646 | 18.61 |
| ATOM | 1157 | HB2 | ASP | 122 | −32.542 | 7.317 | 21.568 |
| ATOM | 1158 | HB1 | ASP | 122 | −30.768 | 7.26 | 21.426 |
| ATOM | 1159 | H8 | ASP | 122 | −31.776 | 5.426 | 23.132 |
| ATOM | 1160 | HA | ASP | 122 | −32.67 | 5.099 | 20.389 |
| ATOM | 1161 | N | ASN | 123 | −30.646 | 3.415 | 20.068 |
| ATOM | 1162 | CA | ASN | 123 | −29.472 | 2.689 | 19.592 |
| ATOM | 1163 | C | ASN | 123 | −29.912 | 1.601 | 18.634 |
| ATOM | 1164 | O | ASN | 123 | −29.549 | 1.657 | 17.47 |
| ATOM | 1165 | CB | ASN | 123 | −28.694 | 2.111 | 20.806 |
| ATOM | 1166 | CG | ASN | 123 | −28.313 | 3.192 | 21.79 |
| ATOM | 1167 | OD1 | ASN | 123 | −28.912 | 3.27 | 22.851 |
| ATOM | 1168 | ND2 | ASN | 123 | −27.323 | 4.052 | 21.475 |
| ATOM | 1169 | H | ASN | 123 | −31.513 | 2.926 | 20.172 |
| ATOM | 1170 | HB2 | ASN | 123 | −27.787 | 1.594 | 20.456 |
| ATOM | 1171 | HB1 | ASN | 123 | −29.308 | 1.377 | 21.346 |
| ATOM | 1172 | HD21 | ASN | 123 | −26.834 | 3.987 | 20.608 |
| ATOM | 1173 | HD22 | ASN | 123 | −27.085 | 4.78 | 22.119 |
| ATOM | 1174 | HA | ASN | 123 | −28.805 | 3.368 | 19.031 |
| ATOM | 1175 | N | VAL | 124 | −30.71 | 0.598 | 19.074 |
| ATOM | 1176 | CA | VAL | 124 | −31.166 | −0.419 | 18.129 |
| ATOM | 1177 | C | VAL | 124 | −32.048 | 0.246 | 17.098 |
| ATOM | 1178 | O | VAL | 124 | −31.919 | −0.077 | 15.929 |
| ATOM | 1179 | CB | VAL | 124 | −31.897 | −1.644 | 18.766 |
| ATOM | 1180 | CG1 | VAL | 124 | −30.979 | −2.373 | 19.787 |
| ATOM | 1181 | CG2 | VAL | 124 | −33.265 | −1.292 | 19.417 |
| ATOM | 1182 | HB | VAL | 124 | −32.108 | −2.356 | 17.947 |
| ATOM | 1183 | HG23 | VAL | 124 | −33.152 | −0.546 | 20.217 |
| ATOM | 1184 | HG22 | VAL | 124 | −33.715 | −2.195 | 19.857 |
| ATOM | 1185 | HG21 | VAL | 124 | −33.973 | −0.904 | 18.668 |
| ATOM | 1186 | HG13 | VAL | 124 | −30.019 | −2.642 | 19.32 |
| ATOM | 1187 | HG12 | VAL | 124 | −31.46 | −3.3 | 20.138 |
| ATOM | 1188 | HG11 | VAL | 124 | −30.776 | −1.742 | 20.664 |
| ATOM | 1189 | HA | VAL | 124 | −30.277 | −0.811 | 17.605 |
| ATOM | 1190 | H | VAL | 124 | −31.036 | 0.559 | 20.017 |
| ATOM | 1191 | N | ILE | 125 | −32.947 | 1.178 | 17.494 |
| ATOM | 1192 | CA | ILE | 125 | −33.829 | 1.792 | 16.502 |
| ATOM | 1193 | C | ILE | 125 | −32.977 | 2.402 | 15.415 |
| ATOM | 1194 | O | ILE | 125 | −33.132 | 2.068 | 14.25 |
| ATOM | 1195 | CB | ILE | 125 | −34.867 | 2.785 | 17.112 |
| ATOM | 1196 | CG1 | ILE | 125 | −36.024 | 3.134 | 16.122 |
| ATOM | 1197 | CG2 | ILE | 125 | −34.239 | 4.066 | 17.733 |
| ATOM | 1198 | CD1 | ILE | 125 | −35.597 | 3.651 | 14.722 |
| ATOM | 1199 | HB | ILE | 125 | −35.339 | 2.248 | 17.956 |
| ATOM | 1200 | HG23 | ILE | 125 | −33.421 | 3.812 | 18.42 |
| ATOM | 1201 | HG22 | ILE | 125 | −34.992 | 4.608 | 18.322 |
| ATOM | 1202 | HG21 | ILE | 125 | −33.858 | 4.755 | 16.966 |
| ATOM | 1203 | HG12 | ILE | 125 | −36.644 | 3.903 | 16.597 |
| ATOM | 1204 | HG11 | ILE | 125 | −36.668 | 2.255 | 15.973 |
| ATOM | 1205 | HD13 | ILE | 125 | −34.74 | 4.335 | 14.773 |
| ATOM | 1206 | HD12 | ILE | 125 | −36.423 | 4.194 | 14.249 |
| ATOM | 1207 | HD11 | ILE | 125 | −35.363 | 2.808 | 14.058 |
| ATOM | 1208 | H | ILE | 125 | −33.031 | 1.443 | 18.454 |
| ATOM | 1209 | HA | ILE | 125 | −34.424 | 0.978 | 16.067 |
| ATOM | 1210 | N | ASP | 126 | −32.035 | 3.303 | 15.753 |
| ATOM | 1211 | CA | ASP | 126 | −31.298 | 3.958 | 14.684 |
| ATOM | 1212 | C | ASP | 126 | −30.511 | 2.916 | 13.95 |
| ATOM | 1213 | O | ASP | 126 | −30.508 | 2.864 | 12.731 |
| ATOM | 1214 | CB | ASP | 126 | −30.295 | 4.999 | 15.222 |
| ATOM | 1215 | CG | ASP | 126 | −29.736 | 5.716 | 14.023 |
| ATOM | 1216 | OD1 | ASP | 126 | −30.342 | 6.755 | 13.64 |
| ATOM | 1217 | OD2 | ASP | 126 | −28.714 | 5.231 | 13.461 |
| ATOM | 1218 | HB2 | ASP | 126 | −30.845 | 5.677 | 15.888 |
| ATOM | 1219 | HB1 | ASP | 126 | −29.481 | 4.535 | 15.802 |
| ATOM | 1220 | H | ASP | 126 | −31.802 | 3.509 | 16.703 |
| ATOM | 1221 | HA | ASP | 126 | −32.011 | 4.451 | 14.002 |
| ATOM | 1222 | N | SER | 127 | −29.801 | 2.048 | 14.69 |
| ATOM | 1223 | CA | SER | 127 | −28.88 | 1.171 | 13.998 |
| ATOM | 1224 | C | SER | 127 | −29.609 | 0.084 | 13.22 |
| ATOM | 1225 | O | SER | 127 | −28.979 | −0.475 | 12.334 |
| ATOM | 1226 | CB | SER | 127 | −27.688 | 0.874 | 14.946 |
| ATOM | 1227 | OG | SER | 127 | −27.286 | 2.083 | 15.646 |
| ATOM | 1228 | HB2 | SER | 127 | −28.016 | 0.147 | 15.705 |
| ATOM | 1229 | HB1 | SER | 127 | −26.843 | 0.434 | 14.393 |
| ATOM | 1230 | HG | SER | 127 | −27.078 | 2.868 | 15.131 |
| ATOM | 1231 | H | SER | 127 | −29.849 | 2.014 | 15.694 |
| ATOM | 1232 | HA | SER | 127 | −28.414 | 1.775 | 13.208 |
| ATOM | 1233 | N | VAL | 128 | −30.921 | −0.212 | 13.441 |
| ATOM | 1234 | CA | VAL | 128 | −31.667 | −0.99 | 12.441 |
| ATOM | 1235 | C | VAL | 128 | −31.94 | −0.112 | 11.235 |
| ATOM | 1236 | O | VAL | 128 | −31.943 | −0.627 | 10.128 |
| ATOM | 1237 | CB | VAL | 128 | −32.996 | −1.707 | 12.84 |
| ATOM | 1238 | CG1 | VAL | 128 | −32.764 | −2.763 | 13.957 |
| ATOM | 1239 | CG2 | VAL | 128 | −34.15 | −0.744 | 13.23 |
| ATOM | 1240 | HB | VAL | 128 | −33.352 | −2.258 | 11.948 |
| ATOM | 1241 | HG23 | VAL | 128 | −33.908 | −0.267 | 14.182 |
| ATOM | 1242 | HG22 | VAL | 128 | −35.093 | −1.299 | 13.359 |
| ATOM | 1243 | HG21 | VAL | 128 | −34.316 | 0.033 | 12.469 |
| ATOM | 1244 | HG13 | VAL | 128 | −32.024 | −3.511 | 13.635 |
| ATOM | 1245 | HG12 | VAL | 128 | −33.704 | −3.287 | 14.192 |
| ATOM | 1246 | HG11 | VAL | 128 | −32.396 | −2.285 | 14.875 |
| ATOM | 1247 | H | VAL | 128 | −31.431 | 0.19 | 14.204 |
| ATOM | 1248 | HA | VAL | 128 | −31.007 | −1.81 | 12.116 |
| ATOM | 1249 | N | ILE | 129 | −32.165 | 1.214 | 11.372 |
| ATOM | 1250 | CA | ILE | 129 | −32.257 | 2.043 | 10.168 |
| ATOM | 1251 | C | ILE | 129 | −30.974 | 1.853 | 9.39 |
| ATOM | 1252 | O | ILE | 129 | −31.03 | 1.665 | 8.184 |
| ATOM | 1253 | CB | ILE | 129 | −32.607 | 3.536 | 10.461 |
| ATOM | 1254 | CG1 | ILE | 129 | −34.092 | 3.641 | 10.914 |
| ATOM | 1255 | CG2 | ILE | 129 | −32.332 | 4.434 | 9.225 |
| ATOM | 1256 | CD1 | ILE | 129 | −34.483 | 5.018 | 11.505 |
| ATOM | 1257 | HB | ILE | 129 | −31.967 | 3.921 | 11.266 |
| ATOM | 1258 | HG23 | ILE | 129 | −31.277 | 4.363 | 8.927 |
| ATOM | 1259 | HG22 | ILE | 129 | −32.529 | 5.489 | 9.459 |
| ATOM | 1260 | HG21 | ILE | 129 | −32.967 | 4.128 | 8.38 |
| ATOM | 1261 | HG12 | ILE | 129 | −34.303 | 2.871 | 11.674 |
| ATOM | 1262 | HG11 | ILE | 129 | −34.73 | 3.452 | 10.04 |
| ATOM | 1263 | HD13 | ILE | 129 | −33.82 | 5.281 | 12.343 |
| ATOM | 1264 | HD12 | ILE | 129 | −35.519 | 4.978 | 11.876 |
| ATOM | 1265 | HD11 | ILE | 129 | −34.423 | 5.807 | 10.743 |
| ATOM | 1266 | H | ILE | 129 | −32.196 | 1.652 | 12.273 |
| ATOM | 1267 | HA | ILE | 129 | −33.054 | 1.639 | 9.528 |
| ATOM | 1268 | N | CYS | 130 | −29.795 | 1.864 | 10.046 |
| ATOM | 1269 | CA | CYS | 130 | −28.576 | 1.565 | 9.297 |
| ATOM | 1270 | C | CYS | 130 | −28.633 | 0.158 | 8.741 |
| ATOM | 1271 | O | CYS | 130 | −28.178 | −0.035 | 7.627 |
| ATOM | 1272 | CB | CYS | 130 | −27.272 | 1.78 | 10.097 |
| ATOM | 1273 | SG | CYS | 130 | −27.09 | 3.566 | 10.455 |
| ATOM | 1274 | HG | CYS | 130 | −27.902 | 3.7 | 11.038 |
| ATOM | 1275 | HB2 | CYS | 130 | −27.293 | 1.159 | 11.003 |
| ATOM | 1276 | HB1 | CYS | 130 | −26.405 | 1.48 | 9.488 |
| ATOM | 1277 | H | CYS | 130 | −29.755 | 2.033 | 11.034 |
| ATOM | 1278 | HA | CYS | 130 | −28.528 | 2.268 | 8.453 |
| ATOM | 1279 | LPG2 | CYS | 130 | −27.118 | 3.942 | 9.89 |
| ATOM | 1280 | LPG1 | CYS | 130 | −26.514 | 3.7 | 10.788 |
| ATOM | 1281 | N | SER | 131 | −29.19 | −0.856 | 9.441 |
| ATOM | 1282 | CA | SER | 131 | −29.383 | −2.149 | 8.777 |
| ATOM | 1283 | C | SER | 131 | −30.135 | −1.931 | 7.482 |
| ATOM | 1284 | O | SER | 131 | −29.727 | −2.467 | 6.464 |
| ATOM | 1285 | CB | SER | 131 | −30.15 | −3.205 | 9.617 |
| ATOM | 1286 | OG | SER | 131 | −30.301 | −4.42 | 8.861 |
| ATOM | 1287 | HB2 | SER | 131 | −29.594 | −3.396 | 10.55 |
| ATOM | 1288 | HB1 | SER | 131 | −31.157 | −2.846 | 9.867 |
| ATOM | 1289 | HG | SER | 131 | −30.758 | −5.107 | 9.337 |
| ATOM | 1290 | H | SER | 131 | −29.497 | −0.727 | 10.385 |
| ATOM | 1291 | HA | SER | 131 | −28.389 | −2.564 | 8.543 |
| ATOM | 1292 | N | SER | 132 | −31.229 | −1.136 | 7.475 |
| ATOM | 1293 | CA | SER | 132 | −31.916 | −0.885 | 6.207 |
| ATOM | 1294 | C | SER | 132 | −30.966 | −0.215 | 5.239 |
| ATOM | 1295 | O | SER | 132 | −30.976 | −0.567 | 4.071 |
| ATOM | 1296 | CB | SER | 132 | −33.216 | −0.051 | 6.345 |
| ATOM | 1297 | OG | SER | 132 | −33.964 | −0.18 | 5.121 |
| ATOM | 1298 | HB2 | SER | 132 | −33.808 | −0.451 | 7.184 |
| ATOM | 1299 | HB1 | SER | 132 | −32.981 | 1.005 | 6.554 |
| ATOM | 1300 | HG | SER | 132 | −34.821 | 0.241 | 5.133 |
| ATOM | 1301 | H | SER | 132 | −31.558 | −0.692 | 8.312 |
| ATOM | 1302 | HA | SER | 132 | −32.203 | −1.862 | 5.788 |
| ATOM | 1303 | N | LEU | 133 | −30.122 | 0.738 | 5.692 |
| ATOM | 1304 | CA | LEU | 133 | −29.123 | 1.323 | 4.791 |
| ATOM | 1305 | C | LEU | 133 | −28.306 | 0.205 | 4.188 |
| ATOM | 1306 | O | LEU | 133 | −28.234 | 0.106 | 2.974 |
| ATOM | 1307 | CB | LEU | 133 | −28.172 | 2.306 | 5.534 |
| ATOM | 1308 | CG | LEU | 133 | −27.33 | 3.27 | 4.649 |
| ATOM | 1309 | CD1 | LEU | 133 | −26.53 | 4.219 | 5.581 |
| ATOM | 1310 | CD2 | LEU | 133 | −26.34 | 2.529 | 3.711 |
| ATOM | 1311 | HB2 | LEU | 133 | −28.806 | 2.924 | 6.179 |
| ATOM | 1312 | HB1 | LEU | 133 | −27.472 | 1.767 | 6.185 |
| ATOM | 1313 | HG | LEU | 133 | −28.014 | 3.883 | 4.038 |
| ATOM | 1314 | HD13 | LEU | 133 | −27.199 | 4.693 | 6.313 |
| ATOM | 1315 | HD12 | LEU | 133 | −26.046 | 5.013 | 4.997 |
| ATOM | 1316 | HD11 | LEU | 133 | −25.756 | 3.657 | 6.127 |
| ATOM | 1317 | HD23 | LEU | 133 | −25.823 | 1.717 | 4.242 |
| ATOM | 1318 | HD22 | LEU | 133 | −25.572 | 3.21 | 3.316 |
| ATOM | 1319 | HD21 | LEU | 133 | −26.886 | 2.112 | 2.859 |
| ATOM | 1320 | H | LEU | 133 | −30.162 | 1.031 | 6.65 |
| ATOM | 1321 | HA | LEU | 133 | −29.656 | 1.877 | 4 |
| ATOM | 1322 | N | LEU | 134 | −27.679 | −0.654 | 5.023 |
| ATOM | 1323 | CA | LEU | 134 | −26.814 | −1.694 | 4.472 |
| ATOM | 1324 | C | LEU | 134 | −27.617 | −2.545 | 3.525 |
| ATOM | 1325 | O | LEU | 134 | −27.159 | −2.78 | 2.419 |
| ATOM | 1326 | CB | LEU | 134 | −26.118 | −2.59 | 5.535 |
| ATOM | 1327 | CG | LEU | 134 | −24.763 | −2.031 | 6.067 |
| ATOM | 1328 | CD1 | LEU | 134 | −23.654 | −2.008 | 4.978 |
| ATOM | 1329 | CD2 | LEU | 134 | −24.921 | −0.638 | 6.731 |
| ATOM | 1330 | HB2 | LEU | 134 | −26.814 | −2.735 | 6.374 |
| ATOM | 1331 | HB1 | LEU | 134 | −25.914 | −3.584 | 5.101 |
| ATOM | 1332 | HG | LEU | 134 | −24.414 | −2.736 | 6.841 |
| ATOM | 1333 | HD13 | LEU | 134 | −23.587 | −2.991 | 4.497 |
| ATOM | 1334 | HD12 | LEU | 134 | −22.681 | −1.806 | 5.435 |
| ATOM | 1335 | HD11 | LEU | 134 | −23.811 | −1.238 | 4.21 |
| ATOM | 1336 | HD23 | LEU | 134 | −25.276 | 0.104 | 6.004 |
| ATOM | 1337 | HD22 | LEU | 134 | −23.954 | −0.29 | 7.123 |
| ATOM | 1338 | HD21 | LEU | 134 | −25.637 | −0.692 | 7.564 |
| ATOM | 1339 | H | LEU | 134 | −27.811 | −0.599 | 6.014 |
| ATOM | 1340 | HA | LEU | 134 | −26.046 | −1.202 | 3.87 |
| ATOM | 1341 | N | ALA | 135 | −28.817 | −3.01 | 3.925 |
| ATOM | 1342 | CA | ALA | 135 | −29.605 | −3.811 | 2.996 |
| ATOM | 1343 | C | ALA | 135 | −29.8 | −3.022 | 1.717 |
| ATOM | 1344 | O | ALA | 135 | −29.597 | −3.583 | 0.654 |
| ATOM | 1345 | CB | ALA | 135 | −30.959 | −4.241 | 3.616 |
| ATOM | 1346 | HB3 | ALA | 135 | −31.573 | −3.358 | 3.84 |
| ATOM | 1347 | HB2 | ALA | 135 | −30.789 | −4.797 | 4.551 |
| ATOM | 1348 | HB1 | ALA | 135 | −31.506 | −4.894 | 2.919 |
| ATOM | 1349 | H | ALA | 135 | −29.183 | −2.797 | 4.833 |
| ATOM | 1350 | HA | ALA | 135 | −29.032 | −4.726 | 2.77 |
| ATOM | 1351 | N | SER | 136 | −30.173 | −1.725 | 1.783 |
| ATOM | 1352 | CA | SER | 136 | −30.371 | −0.932 | 0.567 |
| ATOM | 1353 | C | SER | 136 | −29.103 | −0.792 | −0.245 |
| ATOM | 1354 | O | SER | 136 | −29.075 | −1.238 | −1.382 |
| ATOM | 1355 | CB | SER | 136 | −30.978 | 0.46 | 0.885 |
| ATOM | 1356 | OG | SER | 136 | −32.249 | 0.255 | 1.527 |
| ATOM | 1357 | HB2 | SER | 136 | −30.31 | 1.029 | 1.551 |
| ATOM | 1358 | HB1 | SER | 136 | −31.114 | 1.031 | −0.05 |
| ATOM | 1359 | HG | SER | 136 | −32.693 | 1.064 | 1.76 |
| ATOM | 1360 | H | SER | 136 | −30.317 | −1.282 | 2.669 |
| ATOM | 1361 | HA | SER | 136 | −31.115 | −1.416 | −0.08 |
| ATOM | 1362 | N | ILE | 137 | −28.033 | −0.163 | 0.286 |
| ATOM | 1363 | CA | ILE | 137 | −26.861 | 0.078 | −0.556 |
| ATOM | 1364 | C | ILE | 137 | −26.207 | −1.233 | −0.937 |
| ATOM | 1365 | O | ILE | 137 | −25.762 | −1.365 | −2.068 |
| ATOM | 1366 | CB | ILE | 137 | −25.861 | 1.106 | 0.059 |
| ATOM | 1367 | CG1 | ILE | 137 | −24.88 | 1.695 | −0.998 |
| ATOM | 1368 | CG2 | ILE | 137 | −25.049 | 0.468 | 1.219 |
| ATOM | 1369 | CD1 | ILE | 137 | −25.529 | 2.729 | −1.961 |
| ATOM | 1370 | HB | ILE | 137 | −26.441 | 1.95 | 0.463 |
| ATOM | 1371 | HG23 | ILE | 137 | −25.729 | 0.046 | 1.965 |
| ATOM | 1372 | HG22 | ILE | 137 | −24.415 | 1.216 | 1.721 |
| ATOM | 1373 | HG21 | ILE | 137 | −24.405 | −0.337 | 0.844 |
| ATOM | 1374 | HG12 | ILE | 137 | −24.438 | 0.865 | −1.564 |
| ATOM | 1375 | HG11 | ILE | 137 | −24.06 | 2.214 | −0.475 |
| ATOM | 1376 | HD13 | ILE | 137 | −26.392 | 2.34 | −2.516 |
| ATOM | 1377 | HD12 | ILE | 137 | −24.792 | 3.074 | −2.699 |
| ATOM | 1378 | HD11 | ILE | 137 | −25.863 | 3.598 | −1.384 |
| ATOM | 1379 | H | ILE | 137 | −28.038 | 0.147 | 1.238 |
| ATOM | 1380 | HA | ILE | 137 | −27.249 | 0.516 | −1.482 |
| ATOM | 1381 | N | CYS | 138 | −26.136 | −2.232 | −0.028 |
| ATOM | 1382 | CA | CYS | 138 | −25.528 | −3.504 | −0.41 |
| ATOM | 1383 | C | CYS | 138 | −26.464 | −4.254 | −1.328 |
| ATOM | 1384 | O | CYS | 138 | −25.971 | −4.945 | −2.204 |
| ATOM | 1385 | CB | CYS | 138 | −25.111 | −4.398 | 0.783 |
| ATOM | 1386 | SG | CYS | 138 | −23.851 | −3.533 | 1.773 |
| ATOM | 1387 | HG | CYS | 138 | −24.406 | −2.745 | 2.065 |
| ATOM | 1388 | HB2 | CYS | 138 | −25.976 | −4.672 | 1.401 |
| ATOM | 1389 | HB1 | CYS | 138 | −24.643 | −5.321 | 0.408 |
| ATOM | 1390 | H | CYS | 138 | −26.539 | −2.145 | 0.885 |
| ATOM | 1391 | HA | CYS | 138 | −24.597 | −3.287 | −0.959 |
| ATOM | 1392 | LPG2 | CYS | 138 | −23.317 | −3.345 | 1.399 |
| ATOM | 1393 | LPG1 | CYS | 138 | −23.639 | −3.906 | 2.3 |
| ATOM | 1394 | N | SER | 139 | −27.806 | −4.137 | −1.191 |
| ATOM | 1395 | CA | SER | 139 | −28.67 | −4.694 | −2.229 |
| ATOM | 1396 | C | SER | 139 | −28.261 | −4.046 | −3.528 |
| ATOM | 1397 | O | SER | 139 | −28.029 | −4.754 | −4.491 |
| ATOM | 1398 | CB | SER | 139 | −30.199 | −4.468 | −2.081 |
| ATOM | 1399 | OG | SER | 139 | −30.757 | −5.455 | −1.195 |
| ATOM | 1400 | H | SER | 139 | −28.226 | −3.622 | −0.442 |
| ATOM | 1401 | HB2 | SER | 139 | −30.671 | −4.592 | −3.069 |
| ATOM | 1402 | HB1 | SER | 139 | −30.408 | −3.443 | −1.739 |
| ATOM | 1403 | HG | SER | 139 | −31.702 | −5.397 | −1.107 |
| ATOM | 1404 | HA | SER | 139 | −28.503 | −5.779 | −2.277 |
| ATOM | 1405 | N | LEU | 140 | −28.166 | −2.702 | −3.585 |
| ATOM | 1406 | CA | LEU | 140 | −27.821 | −2.057 | −4.851 |
| ATOM | 1407 | C | LEU | 140 | −26.486 | −2.568 | −5.347 |
| ATOM | 1408 | O | LEU | 140 | −26.346 | −2.76 | −6.545 |
| ATOM | 1409 | CB | LEU | 140 | −27.92 | −0.51 | −4.75 |
| ATOM | 1410 | CG | LEU | 140 | −29.391 | −0.031 | −4.532 |
| ATOM | 1411 | CD1 | LEU | 140 | −29.42 | 1.398 | −3.923 |
| ATOM | 1412 | CD2 | LEU | 140 | −30.216 | −0.049 | −5.85 |
| ATOM | 1413 | HA | LEU | 140 | −28.544 | −2.386 | −5.605 |
| ATOM | 1414 | H | LEU | 140 | −28.318 | −2.144 | −2.764 |
| ATOM | 1415 | HB2 | LEU | 140 | −27.512 | −0.04 | −5.659 |
| ATOM | 1416 | HB1 | LEU | 140 | −27.297 | −0.204 | −3.899 |
| ATOM | 1417 | HG | LEU | 140 | −29.901 | −0.699 | −3.819 |
| ATOM | 1418 | HD13 | LEU | 140 | −29.017 | 1.399 | −2.9 |
| ATOM | 1419 | HD12 | LEU | 140 | −30.448 | 1.792 | −3.884 |
| ATOM | 1420 | HD11 | LEU | 140 | −28.806 | 2.07 | −4.536 |
| ATOM | 1421 | HD23 | LEU | 140 | −29.878 | 0.749 | −6.526 |
| ATOM | 1422 | HD22 | LEU | 140 | −31.275 | 0.113 | −5.61 |
| ATOM | 1423 | HD21 | LEU | 140 | −30.155 | −1.003 | −6.386 |
| ATOM | 1424 | N | LEU | 141 | −25.5 | −2.842 | −4.464 |
| ATOM | 1425 | CA | LEU | 141 | −24.298 | −3.537 | −4.929 |
| ATOM | 1426 | C | LEU | 141 | −24.697 | −4.878 | −5.501 |
| ATOM | 1427 | O | LEU | 141 | −24.27 | −5.209 | −6.597 |
| ATOM | 1428 | CB | LEU | 141 | −23.234 | −3.68 | −3.803 |
| ATOM | 1429 | CG | LEU | 141 | −21.893 | −4.343 | −4.25 |
| ATOM | 1430 | CD1 | LEU | 141 | −20.734 | −3.859 | −3.332 |
| ATOM | 1431 | CD2 | LEU | 141 | −21.943 | −5.899 | −4.2 |
| ATOM | 1432 | HB2 | LEU | 141 | −23.035 | −2.659 | −3.442 |
| ATOM | 1433 | HB1 | LEU | 141 | −23.646 | −4.256 | −2.964 |
| ATOM | 1434 | HG | LEU | 141 | −21.661 | −4.031 | −5.283 |
| ATOM | 1435 | HD13 | LEU | 141 | −20.589 | −2.771 | −3.424 |
| ATOM | 1436 | HD12 | LEU | 141 | −19.791 | −4.353 | −3.61 |
| ATOM | 1437 | HD11 | LEU | 141 | −20.955 | −4.1 | −2.281 |
| ATOM | 1438 | HD23 | LEU | 141 | −22.141 | −6.243 | −3.174 |
| ATOM | 1439 | HD22 | LEU | 141 | −20.979 | −6.321 | −4.525 |
| ATOM | 1440 | HD21 | LEU | 141 | −22.716 | −6.317 | −4.855 |
| ATOM | 1441 | H | LEU | 141 | −25.608 | −2.631 | −3.489 |
| ATOM | 1442 | HA | LEU | 141 | −23.845 | −2.945 | −5.738 |
| ATOM | 1443 | N | SER | 142 | −25.516 | −5.685 | −4.791 |
| ATOM | 1444 | CA | SER | 142 | −25.886 | −6.982 | −5.346 |
| ATOM | 1445 | C | SER | 142 | −26.602 | −6.819 | −6.669 |
| ATOM | 1446 | O | SER | 142 | −26.377 | −7.624 | −7.56 |
| ATOM | 1447 | CB | SER | 142 | −26.725 | −7.877 | −4.396 |
| ATOM | 1448 | OG | SER | 142 | −28.007 | −7.332 | −4.056 |
| ATOM | 1449 | HB2 | SER | 142 | −26.864 | −8.86 | −4.874 |
| ATOM | 1450 | HB1 | SER | 142 | −26.172 | −8.022 | −3.455 |
| ATOM | 1451 | HG | SER | 142 | −28.549 | −7.134 | −4.812 |
| ATOM | 1452 | H | SER | 142 | −25.887 | −5.414 | −3.901 |
| ATOM | 1453 | HA | SER | 142 | −24.946 | −7.528 | −5.508 |
| ATOM | 1454 | N | ILE | 143 | −27.462 | −5.786 | −6.831 |
| ATOM | 1455 | CA | ILE | 143 | −28.117 | −5.588 | −8.12 |
| ATOM | 1456 | C | ILE | 143 | −27.023 | −5.236 | −9.104 |
| ATOM | 1457 | O | ILE | 143 | −27.05 | −5.717 | −10.223 |
| ATOM | 1458 | CB | ILE | 143 | −29.262 | −4.524 | −8.171 |
| ATOM | 1459 | CG1 | ILE | 143 | −30.253 | −4.696 | −6.977 |
| ATOM | 1460 | CG2 | ILE | 143 | −30.01 | −4.641 | −9.535 |
| ATOM | 1461 | CD1 | ILE | 143 | −31.63 | −3.997 | −7.149 |
| ATOM | 1462 | HB | ILE | 143 | −28.808 | −3.522 | −8.102 |
| ATOM | 1463 | HG23 | ILE | 143 | −29.316 | −4.663 | −10.386 |
| ATOM | 1464 | HG22 | ILE | 143 | −30.684 | −3.786 | −9.691 |
| ATOM | 1465 | HG21 | ILE | 143 | −30.6 | −5.569 | −9.568 |
| ATOM | 1466 | HG12 | ILE | 143 | −29.821 | −4.283 | −6.06 |
| ATOM | 1467 | HG11 | ILE | 143 | −30.401 | −5.765 | −6.796 |
| ATOM | 1468 | HD13 | ILE | 143 | −31.494 | −2.932 | −7.395 |
| ATOM | 1469 | HD12 | ILE | 143 | −32.207 | −4.066 | −6.213 |
| ATOM | 1470 | HD11 | ILE | 143 | −32.226 | −4.473 | −7.941 |
| ATOM | 1471 | H | ILE | 143 | −27.606 | −5.117 | −6.104 |
| ATOM | 1472 | HA | ILE | 143 | −28.581 | −6.547 | −8.39 |
| ATOM | 1473 | N | ALA | 144 | −26.037 | −4.398 | −8.717 |
| ATOM | 1474 | CA | ALA | 144 | −24.976 | −4.031 | −9.653 |
| ATOM | 1475 | C | ALA | 144 | −24.21 | −5.244 | −10.142 |
| ATOM | 1476 | O | ALA | 144 | −23.999 | −5.347 | −11.341 |
| ATOM | 1477 | CB | ALA | 144 | −24.013 | −2.999 | −9.01 |
| ATOM | 1478 | H | ALA | 144 | −26.024 | −4.005 | −7.795 |
| ATOM | 1479 | HB3 | ALA | 144 | −23.425 | −3.466 | −8.208 |
| ATOM | 1480 | HB2 | ALA | 144 | −24.578 | −2.152 | −8.592 |
| ATOM | 1481 | HB1 | ALA | 144 | −23.318 | −2.601 | −9.759 |
| ATOM | 1482 | HA | ALA | 144 | −25.448 | −3.55 | −10.527 |
| ATOM | 1483 | N | VAL | 145 | −23.781 | −6.186 | −9.267 |
| ATOM | 1484 | CA | VAL | 145 | −23.054 | −7.358 | −9.773 |
| ATOM | 1485 | C | VAL | 145 | −24.005 | −8.249 | −10.541 |
| ATOM | 1486 | O | VAL | 145 | −23.624 | −8.761 | −11.582 |
| ATOM | 1487 | CB | VAL | 145 | −22.222 | −8.168 | −8.732 |
| ATOM | 1488 | CG1 | VAL | 145 | −21.291 | −7.235 | −7.908 |
| ATOM | 1489 | CG2 | VAL | 145 | −23.11 | −9.037 | −7.805 |
| ATOM | 1490 | HB | VAL | 145 | −21.577 | −8.872 | −9.289 |
| ATOM | 1491 | HG23 | VAL | 145 | −23.831 | −8.397 | −7.291 |
| ATOM | 1492 | HG22 | VAL | 145 | −22.497 | −9.553 | −7.049 |
| ATOM | 1493 | HG21 | VAL | 145 | −23.661 | −9.8 | −8.375 |
| ATOM | 1494 | HG13 | VAL | 145 | −20.638 | −6.656 | −8.579 |
| ATOM | 1495 | HG12 | VAL | 145 | −20.65 | −7.828 | −7.237 |
| ATOM | 1496 | HG11 | VAL | 145 | −21.869 | −6.531 | −7.294 |
| ATOM | 1497 | H | VAL | 145 | −23.998 | −6.105 | −8.291 |
| ATOM | 1498 | HA | VAL | 145 | −22.314 | −6.992 | −10.502 |
| ATOM | 1499 | N | ASP | 146 | −25.261 | −8.434 | −10.073 |
| ATOM | 1500 | CA | ASP | 146 | −26.242 | −9.129 | −10.909 |
| ATOM | 1501 | C | ASP | 146 | −26.257 | −8.447 | −12.257 |
| ATOM | 1502 | O | ASP | 146 | −26.249 | −9.13 | −13.266 |
| ATOM | 1503 | CB | ASP | 146 | −27.656 | −9.085 | −10.271 |
| ATOM | 1504 | CG | ASP | 146 | −28.778 | −9.541 | −11.167 |
| ATOM | 1505 | OD1 | ASP | 146 | −29.797 | −8.801 | −11.267 |
| ATOM | 1506 | OD2 | ASP | 146 | −28.664 | −10.637 | −11.773 |
| ATOM | 1507 | HB2 | ASP | 146 | −27.662 | −9.69 | −9.352 |
| ATOM | 1508 | HB1 | ASP | 146 | −27.872 | −8.047 | −10.001 |
| ATOM | 1509 | H | ASP | 146 | −25.554 | −8.044 | −9.196 |
| ATOM | 1510 | HA | ASP | 146 | −25.943 | −10.182 | −11.041 |
| ATOM | 1511 | N | ARG | 147 | −26.256 | −7.097 | −12.315 |
| ATOM | 1512 | CA | ARG | 147 | −26.312 | −6.417 | −13.609 |
| ATOM | 1513 | C | ARG | 147 | −25.029 | −6.601 | −14.387 |
| ATOM | 1514 | O | ARG | 147 | −25.093 | −6.658 | −15.605 |
| ATOM | 1515 | CB | ARG | 147 | −26.667 | −4.906 | −13.454 |
| ATOM | 1516 | CG | ARG | 147 | −27.568 | −4.333 | −14.59 |
| ATOM | 1517 | CD | ARG | 147 | −28.822 | −5.183 | −14.99 |
| ATOM | 1518 | NE | ARG | 147 | −29.462 | −5.962 | −13.919 |
| ATOM | 1519 | CZ | ARG | 147 | −30.428 | −6.83 | −14.127 |
| ATOM | 1520 | NH1 | ARG | 147 | −30.987 | −7.007 | −15.299 |
| ATOM | 1521 | NH2 | ARG | 147 | −30.855 | −7.561 | −13.125 |
| ATOM | 1522 | H | ARG | 147 | −26.207 | −6.558 | −11.475 |
| ATOM | 1523 | HB2 | ARG | 147 | −25.734 | −4.322 | −13.382 |
| ATOM | 1524 | HB1 | ARG | 147 | −27.199 | −4.728 | −12.509 |
| ATOM | 1525 | HG2 | ARG | 147 | −26.945 | −4.173 | −15.488 |
| ATOM | 1526 | HG1 | ARG | 147 | −27.905 | −3.338 | −14.254 |
| ATOM | 1527 | HD2 | ARG | 147 | −28.486 | −5.889 | −15.769 |
| ATOM | 1528 | HD1 | ARG | 147 | −29.566 | −4.509 | −15.444 |
| ATOM | 1529 | HE | ARG | 147 | −29.111 | −5.848 | −12.959 |
| ATOM | 1530 | HH11 | ARG | 147 | −30.704 | −6.462 | −16.125 |
| ATOM | 1531 | HH12 | ARG | 147 | −31.733 | −7.703 | −15.428 |
| ATOM | 1532 | HH21 | ARG | 147 | −30.419 | −7.504 | −12.188 |
| ATOM | 1533 | HH22 | ARG | 147 | −31.601 | −8.259 | −13.246 |
| ATOM | 1534 | HA | ARG | 147 | −27.096 | −6.932 | −14.179 |
| ATOM | 1535 | N | TYR | 148 | −23.855 | −6.704 | −13.723 |
| ATOM | 1536 | CA | TYR | 148 | −22.627 | −7.015 | −14.457 |
| ATOM | 1537 | C | TYR | 148 | −22.824 | −8.283 | −15.255 |
| ATOM | 1538 | O | TYR | 148 | −22.476 | −8.309 | −16.425 |
| ATOM | 1539 | CB | TYR | 148 | −21.411 | −7.208 | −13.509 |
| ATOM | 1540 | CG | TYR | 148 | −20.105 | −7.416 | −14.291 |
| ATOM | 1541 | CD1 | TYR | 148 | −19.188 | −6.37 | −14.441 |
| ATOM | 1542 | CD2 | TYR | 148 | −19.811 | −8.662 | −14.857 |
| ATOM | 1543 | CE1 | TYR | 148 | −18.015 | −6.548 | −15.18 |
| ATOM | 1544 | CE2 | TYR | 148 | −18.648 | −8.838 | −15.612 |
| ATOM | 1545 | CZ | TYR | 148 | −17.747 | −7.784 | −15.783 |
| ATOM | 1546 | OH | TYR | 148 | −16.595 | −7.986 | −16.55 |
| ATOM | 1547 | H | TYR | 148 | −23.818 | −6.593 | −12.728 |
| ATOM | 1548 | HB1 | TYR | 148 | −21.562 | −8.077 | −12.852 |
| ATOM | 1549 | HB2 | TYR | 148 | −21.319 | −6.322 | −12.868 |
| ATOM | 1550 | HD1 | TYR | 148 | −19.378 | −5.408 | −13.98 |
| ATOM | 1551 | HD2 | TYR | 148 | −20.483 | −9.502 | −14.714 |
| ATOM | 1552 | HE1 | TYR | 148 | −17.319 | −5.721 | −15.28 |
| ATOM | 1553 | HE2 | TYR | 148 | −18.438 | −9.799 | −16.069 |
| ATOM | 1554 | HH | TYR | 148 | −16.054 | −7.212 | −16.656 |
| ATOM | 1555 | HA | TYR | 148 | −22.403 | −6.185 | −15.145 |
| ATOM | 1556 | N | PHE | 149 | −23.371 | −9.352 | −14.632 |
| ATOM | 1557 | CA | PHE | 149 | −23.567 | −10.604 | −15.364 |
| ATOM | 1558 | C | PHE | 149 | −24.829 | −10.531 | −16.196 |
| ATOM | 1559 | O | PHE | 149 | −24.772 | −10.699 | −17.403 |
| ATOM | 1560 | CB | PHE | 149 | −23.626 | −11.809 | −14.386 |
| ATOM | 1561 | CG | PHE | 149 | −23.95 | −13.092 | −15.163 |
| ATOM | 1562 | CD1 | PHE | 149 | −22.924 | −13.828 | −15.766 |
| ATOM | 1563 | CD2 | PHE | 149 | −25.273 | −13.536 | −15.277 |
| ATOM | 1564 | CE1 | PHE | 149 | −23.215 | −14.998 | −16.472 |
| ATOM | 1565 | CE2 | PHE | 149 | −25.567 | −14.704 | −15.986 |
| ATOM | 1566 | CZ | PHE | 149 | −24.537 | −15.438 | −16.582 |
| ATOM | 1567 | HB2 | PHE | 149 | −22.656 | −11.911 | −13.874 |
| ATOM | 1568 | HB1 | PHE | 149 | −24.387 | −11.651 | −13.607 |
| ATOM | 1569 | HD1 | PHE | 149 | −21.895 | −13.491 | −15.691 |
| ATOM | 1570 | HD2 | PHE | 149 | −26.081 | −12.976 | −14.817 |
| ATOM | 1571 | HE1 | PHE | 149 | −22.415 | −15.567 | −16.936 |
| ATOM | 1572 | HE2 | PHE | 149 | −26.595 | −15.042 | −16.073 |
| ATOM | 1573 | HZ | PHE | 149 | −24.765 | −16.347 | −17.131 |
| ATOM | 1574 | H | PHE | 149 | −23.679 | −9.287 | −13.678 |
| ATOM | 1575 | HA | PHE | 149 | −22.715 | −10.782 | −16.043 |
| ATOM | 1576 | N | THR | 150 | −25.993 | −10.288 | −15.558 |
| ATOM | 1577 | CA | THR | 150 | −27.253 | −10.215 | −16.292 |
| ATOM | 1578 | C | THR | 150 | −27.223 | −9.01 | −17.195 |
| ATOM | 1579 | O | THR | 150 | −27.582 | −9.091 | −18.359 |
| ATOM | 1580 | CB | THR | 150 | −28.427 | −10.103 | −15.279 |
| ATOM | 1581 | OG1 | THR | 150 | −28.26 | −11.179 | −14.339 |
| ATOM | 1582 | CG2 | THR | 150 | −29.81 | −10.199 | −15.971 |
| ATOM | 1583 | HB | THR | 150 | −28.351 | −9.133 | −14.755 |
| ATOM | 1584 | HG22 | THR | 150 | −30.61 | −10.122 | −15.218 |
| ATOM | 1585 | HG21 | THR | 150 | −29.907 | −11.166 | −16.486 |
| ATOM | 1586 | HG23 | THR | 150 | −29.936 | −9.39 | −16.706 |
| ATOM | 1587 | HG1 | THR | 150 | −28.817 | −11.101 | −13.566 |
| ATOM | 1588 | H | THR | 150 | −26.015 | −10.154 | −14.568 |
| ATOM | 1589 | HA | THR | 150 | −27.377 | −11.137 | −16.883 |
| ATOM | 1590 | H6 | THR | 150 | −26.882 | −8.052 | −16.809 |
| TER | | | THR | 150 | | | |
| ATOM | 1591 | CA | VAL | 163 | −32.898 | −19.944 | −5.256 |
| ATOM | 1592 | C | VAL | 163 | −31.593 | −19.843 | −4.496 |
| ATOM | 1593 | O | VAL | 163 | −31.517 | −19.075 | −3.551 |
| ATOM | 1594 | CB | VAL | 163 | −33.784 | −21.125 | −4.763 |
| ATOM | 1595 | CG1 | VAL | 163 | −35.136 | −21.131 | −5.53 |
| ATOM | 1596 | CG2 | VAL | 163 | −33.078 | −22.5 | −4.918 |
| ATOM | 1597 | HB | VAL | 163 | −34 | −20.97 | −3.69 |
| ATOM | 1598 | HG23 | VAL | 163 | −32.781 | −22.664 | −5.965 |
| ATOM | 1599 | HG22 | VAL | 163 | −33.757 | −23.313 | −4.617 |
| ATOM | 1600 | HG21 | VAL | 163 | −32.185 | −22.559 | −4.278 |
| ATOM | 1601 | HG13 | VAL | 163 | −35.664 | −20.173 | −5.396 |
| ATOM | 1602 | HG12 | VAL | 163 | −35.789 | −21.935 | −5.158 |
| ATOM | 1603 | HG11 | VAL | 163 | −34.966 | −21.288 | −6.606 |
| ATOM | 1604 | HA | VAL | 163 | −33.471 | −19.016 | −5.111 |
| ATOM | 1605 | H7 | VAL | 163 | −32.693 | −20.044 | −6.333 |
| ATOM | 1606 | N | LYS | 164 | −30.548 | −20.616 | −4.871 |
| ATOM | 1607 | CA | LYS | 164 | −29.338 | −20.653 | −4.047 |
| ATOM | 1608 | C | LYS | 164 | −28.532 | −19.378 | −4.154 |
| ATOM | 1609 | O | LYS | 164 | −28.138 | −18.858 | −3.122 |
| ATOM | 1610 | CB | LYS | 164 | −28.468 | −21.918 | −4.311 |
| ATOM | 1611 | CG | LYS | 164 | −27.744 | −21.947 | −5.688 |
| ATOM | 1612 | CD | LYS | 164 | −27.049 | −23.319 | −5.917 |
| ATOM | 1613 | CE | LYS | 164 | −26.334 | −23.388 | −7.295 |
| ATOM | 1614 | NZ | LYS | 164 | −25.721 | −24.72 | −7.524 |
| ATOM | 1615 | HB2 | LYS | 164 | −27.701 | −21.993 | −3.521 |
| ATOM | 1616 | HB1 | LYS | 164 | −29.128 | −22.797 | −4.226 |
| ATOM | 1617 | HG2 | LYS | 164 | −26.984 | −21.149 | −5.724 |
| ATOM | 1618 | HG1 | LYS | 164 | −28.462 | −21.78 | −6.504 |
| ATOM | 1619 | HD2 | LYS | 164 | −27.806 | −24.119 | −5.872 |
| ATOM | 1620 | HD1 | LYS | 164 | −26.314 | −23.496 | −5.115 |
| ATOM | 1621 | HE2 | LYS | 164 | −25.557 | −22.604 | −7.337 |
| ATOM | 1622 | HE1 | LYS | 164 | −27.067 | −23.18 | −8.095 |
| ATOM | 1623 | HZ3 | LYS | 164 | −24.985 | −24.964 | −6.769 |
| ATOM | 1624 | HZ2 | LYS | 164 | −25.217 | −24.773 | −8.481 |
| ATOM | 1625 | HZ1 | LYS | 164 | −26.456 | −25.514 | −7.52 |
| ATOM | 1626 | H | LYS | 164 | −30.608 | −21.196 | −5.683 |
| ATOM | 1627 | HA | LYS | 164 | −29.67 | −20.741 | −2.996 |
| ATOM | 1628 | N | ARG | 165 | −28.262 | −18.832 | −5.362 |
| ATOM | 1629 | CA | ARG | 165 | −27.459 | −17.61 | −5.426 |
| ATOM | 1630 | C | ARG | 165 | −28.212 | −16.505 | −4.726 |
| ATOM | 1631 | O | ARG | 165 | −27.653 | −15.882 | −3.838 |
| ATOM | 1632 | CB | ARG | 165 | −27.114 | −17.155 | −6.872 |
| ATOM | 1633 | CG | ARG | 165 | −26.123 | −18.094 | −7.621 |
| ATOM | 1634 | CD | ARG | 165 | −24.69 | −18.109 | −7.011 |
| ATOM | 1635 | NE | ARG | 165 | −23.716 | −18.768 | −7.888 |
| ATOM | 1636 | CZ | ARG | 165 | −22.431 | −18.858 | −7.615 |
| ATOM | 1637 | NH1 | ARG | 165 | −21.903 | −18.379 | −6.513 |
| ATOM | 1638 | NH2 | ARG | 165 | −21.639 | −19.451 | −8.477 |
| ATOM | 1639 | HB2 | ARG | 165 | −28.05 | −17.089 | −7.451 |
| ATOM | 1640 | HB1 | ARG | 165 | −26.678 | −16.142 | −6.834 |
| ATOM | 1641 | HG2 | ARG | 165 | −26.046 | −17.722 | −8.657 |
| ATOM | 1642 | HG1 | ARG | 165 | −26.522 | −19.121 | −7.659 |
| ATOM | 1643 | HD2 | ARG | 165 | −24.715 | −18.643 | −6.048 |
| ATOM | 1644 | HD1 | ARG | 165 | −24.384 | −17.062 | −6.853 |
| ATOM | 1645 | HE | ARG | 165 | −24.071 | −19.17 | −8.771 |
| ATOM | 1646 | HH21 | ARG | 165 | −22.003 | −19.84 | −9.36 |
| ATOM | 1647 | HH22 | ARG | 165 | −20.628 | −19.541 | −8.3 |
| ATOM | 1648 | HH11 | ARG | 165 | −22.472 | −17.908 | −5.799 |
| ATOM | 1649 | HH12 | ARG | 165 | −20.893 | −18.463 | −6.329 |
| ATOM | 1650 | H | ARG | 165 | −28.63 | −19.227 | −6.204 |
| ATOM | 1651 | HA | ARG | 165 | −26.524 | −17.765 | −4.864 |
| ATOM | 1652 | N | VAL | 166 | −29.486 | −16.248 | −5.093 |
| ATOM | 1653 | CA | VAL | 166 | −30.238 | −15.216 | −4.379 |
| ATOM | 1654 | C | VAL | 166 | −30.202 | −15.537 | −2.899 |
| ATOM | 1655 | O | VAL | 166 | −30.078 | −14.616 | −2.106 |
| ATOM | 1656 | CB | VAL | 166 | −31.69 | −14.97 | −4.888 |
| ATOM | 1657 | CG1 | VAL | 166 | −31.685 | −14.471 | −6.361 |
| ATOM | 1658 | CG2 | VAL | 166 | −32.595 | −16.221 | −4.742 |
| ATOM | 1659 | HB | VAL | 166 | −32.13 | −14.171 | −4.266 |
| ATOM | 1660 | HG23 | VAL | 166 | −32.188 | −17.047 | −5.337 |
| ATOM | 1661 | HG22 | VAL | 166 | −33.609 | −16.008 | −5.108 |
| ATOM | 1662 | HG21 | VAL | 166 | −32.675 | −16.534 | −3.691 |
| ATOM | 1663 | HG13 | VAL | 166 | −31.121 | −13.529 | −6.449 |
| ATOM | 1664 | HG12 | VAL | 166 | −32.715 | −14.292 | −6.7 |
| ATOM | 1665 | HG11 | VAL | 166 | −31.235 | −15.21 | −7.039 |
| ATOM | 1666 | H | VAL | 166 | −29.921 | −16.776 | −5.82 |
| ATOM | 1667 | HA | VAL | 166 | −29.691 | −14.272 | −4.527 |
| ATOM | 1668 | N | GLY | 167 | −30.274 | −16.833 | −2.512 |
| ATOM | 1669 | CA | GLY | 167 | −30.096 | −17.199 | −1.108 |
| ATOM | 1670 | C | GLY | 167 | −28.811 | −16.625 | −0.556 |
| ATOM | 1671 | O | GLY | 167 | −28.815 | −16.04 | 0.516 |
| ATOM | 1672 | H | GLY | 167 | −30.424 | −17.573 | −3.173 |
| ATOM | 1673 | HA1 | GLY | 167 | −30.969 | −16.869 | −0.528 |
| ATOM | 1674 | HA2 | GLY | 167 | −30.024 | −18.293 | −0.999 |
| ATOM | 1675 | N | ILE | 168 | −27.682 | −16.791 | −1.283 |
| ATOM | 1676 | CA | ILE | 168 | −26.419 | −16.231 | −0.806 |
| ATOM | 1677 | C | ILE | 168 | −26.53 | −14.724 | −0.751 |
| ATOM | 1678 | O | ILE | 168 | −26.061 | −14.146 | 0.217 |
| ATOM | 1679 | CB | ILE | 168 | −25.181 | −16.62 | −1.675 |
| ATOM | 1680 | CG1 | ILE | 168 | −24.94 | −18.161 | −1.774 |
| ATOM | 1681 | CG2 | ILE | 168 | −23.901 | −15.882 | −1.182 |
| ATOM | 1682 | CD1 | ILE | 168 | −24.724 | −18.886 | −0.418 |
| ATOM | 1683 | HB | ILE | 168 | −25.37 | −16.264 | −2.703 |
| ATOM | 1684 | HG23 | ILE | 168 | −23.983 | −14.796 | −1.346 |
| ATOM | 1685 | HG22 | ILE | 168 | −23.013 | −16.23 | −1.732 |
| ATOM | 1686 | HG21 | ILE | 168 | −23.736 | −16.052 | −0.108 |
| ATOM | 1687 | HG12 | ILE | 168 | −24.052 | −18.338 | −2.403 |
| ATOM | 1688 | HG11 | ILE | 168 | −25.785 | −18.649 | −2.281 |
| ATOM | 1689 | HD13 | ILE | 168 | −23.889 | −18.446 | 0.145 |
| ATOM | 1690 | HD12 | ILE | 168 | −24.488 | −19.947 | −0.602 |
| ATOM | 1691 | HD11 | ILE | 168 | −25.632 | −18.852 | 0.202 |
| ATOM | 1692 | H | ILE | 168 | −27.713 | −17.261 | −2.167 |
| ATOM | 1693 | HA | ILE | 168 | −26.248 | −16.578 | 0.225 |
| ATOM | 1694 | N | ILE | 169 | −27.124 | −14.051 | −1.762 |
| ATOM | 1695 | CA | ILE | 169 | −27.171 | −12.588 | −1.717 |
| ATOM | 1696 | C | ILE | 169 | −27.941 | −12.17 | −0.485 |
| ATOM | 1697 | O | ILE | 169 | −27.404 | −11.431 | 0.327 |
| ATOM | 1698 | CB | ILE | 169 | −27.779 | −11.917 | −2.991 |
| ATOM | 1699 | CG1 | ILE | 169 | −26.751 | −11.81 | −4.161 |
| ATOM | 1700 | CG2 | ILE | 169 | −28.32 | −10.49 | −2.677 |
| ATOM | 1701 | CD1 | ILE | 169 | −26.183 | −13.165 | −4.655 |
| ATOM | 1702 | HB | ILE | 169 | −28.645 | −12.507 | −3.323 |
| ATOM | 1703 | HG23 | ILE | 169 | −29.152 | −10.5 | −1.957 |
| ATOM | 1704 | HG22 | ILE | 169 | −28.702 | −10.014 | −3.589 |
| ATOM | 1705 | HG21 | ILE | 169 | −27.512 | −9.866 | −2.273 |
| ATOM | 1706 | HG12 | ILE | 169 | −25.91 | −11.165 | −3.854 |
| ATOM | 1707 | HG11 | ILE | 169 | −27.242 | −11.337 | −5.028 |
| ATOM | 1708 | HD13 | ILE | 169 | −25.486 | −12.995 | −5.491 |
| ATOM | 1709 | HD12 | ILE | 169 | −27.002 | −13.801 | −5.019 |
| ATOM | 1710 | HD11 | ILE | 169 | −25.633 | −13.686 | −3.858 |
| ATOM | 1711 | H | ILE | 169 | −27.54 | −14.544 | −2.53 |
| ATOM | 1712 | HA | ILE | 169 | −26.142 | −12.208 | −1.604 |
| ATOM | 1713 | N | ILE | 170 | −29.212 | −12.608 | −0.337 |
| ATOM | 1714 | CA | ILE | 170 | −30.013 | −12.106 | 0.779 |
| ATOM | 1715 | C | ILE | 170 | −29.301 | −12.356 | 2.091 |
| ATOM | 1716 | O | ILE | 170 | −29.264 | −11.472 | 2.934 |
| ATOM | 1717 | CB | ILE | 170 | −31.492 | −12.601 | 0.802 |
| ATOM | 1718 | CG1 | ILE | 170 | −32.295 | −11.851 | 1.908 |
| ATOM | 1719 | CG2 | ILE | 170 | −31.6 | −14.137 | 1.009 |
| ATOM | 1720 | CD1 | ILE | 170 | −33.835 | −11.925 | 1.718 |
| ATOM | 1721 | HB | ILE | 170 | −31.924 | −12.349 | −0.182 |
| ATOM | 1722 | HG23 | ILE | 170 | −30.984 | −14.657 | 0.27 |
| ATOM | 1723 | HG22 | ILE | 170 | −32.635 | −14.484 | 0.878 |
| ATOM | 1724 | HG21 | ILE | 170 | −31.258 | −14.433 | 2.012 |
| ATOM | 1725 | HG12 | ILE | 170 | −32.01 | −10.788 | 1.928 |
| ATOM | 1726 | HG11 | ILE | 170 | −32.021 | −12.28 | 2.882 |
| ATOM | 1727 | HD13 | ILE | 170 | −34.121 | −11.543 | 0.726 |
| ATOM | 1728 | HD12 | ILE | 170 | −34.339 | −11.31 | 2.48 |
| ATOM | 1729 | HD11 | ILE | 170 | −34.203 | −12.956 | 1.819 |
| ATOM | 1730 | H | ILE | 170 | −29.623 | −13.224 | −1.014 |
| ATOM | 1731 | HA | ILE | 170 | −30.063 | −11.018 | 0.62 |
| ATOM | 1732 | N | SER | 171 | −28.707 | −13.554 | 2.284 |
| ATOM | 1733 | CA | SER | 171 | −27.971 | −13.8 | 3.523 |
| ATOM | 1734 | C | SER | 171 | −26.76 | −12.898 | 3.612 |
| ATOM | 1735 | O | SER | 171 | −26.492 | −12.354 | 4.671 |
| ATOM | 1736 | CB | SER | 171 | −27.505 | −15.276 | 3.596 |
| ATOM | 1737 | OG | SER | 171 | −28.668 | −16.121 | 3.542 |
| ATOM | 1738 | HB2 | SER | 171 | −26.834 | −15.494 | 2.748 |
| ATOM | 1739 | HB1 | SER | 171 | −26.954 | −15.445 | 4.537 |
| ATOM | 1740 | HG | SER | 171 | −28.454 | −17.049 | 3.559 |
| ATOM | 1741 | H | SER | 171 | −28.734 | −14.274 | 1.583 |
| ATOM | 1742 | HA | SER | 171 | −28.635 | −13.61 | 4.382 |
| ATOM | 1743 | N | CYS | 172 | −26.001 | −12.734 | 2.506 |
| ATOM | 1744 | CA | CYS | 172 | −24.781 | −11.934 | 2.567 |
| ATOM | 1745 | C | CYS | 172 | −25.097 | −10.504 | 2.935 |
| ATOM | 1746 | O | CYS | 172 | −24.436 | −9.956 | 3.803 |
| ATOM | 1747 | CB | CYS | 172 | −24.022 | −11.942 | 1.213 |
| ATOM | 1748 | SG | CYS | 172 | −22.526 | −10.902 | 1.308 |
| ATOM | 1749 | HG | CYS | 172 | −22.052 | −11.386 | 2.056 |
| ATOM | 1750 | HB2 | CYS | 172 | −23.731 | −12.97 | 0.943 |
| ATOM | 1751 | HB1 | CYS | 172 | −24.66 | −11.536 | 0.415 |
| ATOM | 1752 | H | CYS | 172 | −26.255 | −13.163 | 1.637 |
| ATOM | 1753 | HA | CYS | 172 | −24.12 | −12.368 | 3.336 |
| ATOM | 1754 | LPG2 | CYS | 172 | −22.671 | −10.259 | 1.472 |
| ATOM | 1755 | LPG1 | CYS | 172 | −22.169 | −10.913 | 0.73 |
| ATOM | 1756 | N | ILE | 173 | −26.097 | −9.861 | 2.295 |
| ATOM | 1757 | CA | ILE | 173 | −26.371 | −8.469 | 2.647 |
| ATOM | 1758 | C | ILE | 173 | −26.849 | −8.4 | 4.08 |
| ATOM | 1759 | O | ILE | 173 | −26.46 | −7.472 | 4.771 |
| ATOM | 1760 | CB | ILE | 173 | −27.297 | −7.699 | 1.658 |
| ATOM | 1761 | CG1 | ILE | 173 | −28.763 | −8.214 | 1.706 |
| ATOM | 1762 | CG2 | ILE | 173 | −26.692 | −7.751 | 0.225 |
| ATOM | 1763 | CD1 | ILE | 173 | −29.69 | −7.502 | 0.688 |
| ATOM | 1764 | HB | ILE | 173 | −27.314 | −6.644 | 1.983 |
| ATOM | 1765 | HG23 | ILE | 173 | −25.608 | −7.569 | 0.242 |
| ATOM | 1766 | HG22 | ILE | 173 | −27.141 | −6.987 | −0.426 |
| ATOM | 1767 | HG21 | ILE | 173 | −26.859 | −8.739 | −0.225 |
| ATOM | 1768 | HG12 | ILE | 173 | −29.176 | −8.059 | 2.717 |
| ATOM | 1769 | HG11 | ILE | 173 | −28.776 | −9.29 | 1.499 |
| ATOM | 1770 | HD13 | ILE | 173 | −29.666 | −6.416 | 0.865 |
| ATOM | 1771 | HD12 | ILE | 173 | −30.724 | −7.855 | 0.799 |
| ATOM | 1772 | HD11 | ILE | 173 | −29.388 | −7.708 | −0.35 |
| ATOM | 1773 | H | ILE | 173 | −26.651 | −10.333 | 1.606 |
| ATOM | 1774 | HA | ILE | 173 | −25.404 | −7.937 | 2.622 |
| ATOM | 1775 | N | TRP | 174 | −27.659 | −9.36 | 4.585 |
| ATOM | 1776 | CA | TRP | 174 | −27.962 | −9.318 | 6.014 |
| ATOM | 1777 | C | TRP | 174 | −26.668 | −9.437 | 6.792 |
| ATOM | 1778 | O | TRP | 174 | −26.506 | −8.722 | 7.767 |
| ATOM | 1779 | CB | TRP | 174 | −28.935 | −10.412 | 6.524 |
| ATOM | 1780 | CG | TRP | 174 | −30.364 | −10.25 | 6.067 |
| ATOM | 1781 | CD1 | TRP | 174 | −31.09 | −11.174 | 5.416 |
| ATOM | 1782 | CD2 | TRP | 174 | −31.266 | −9.056 | 6.281 |
| ATOM | 1783 | NE1 | TRP | 174 | −32.297 | −10.717 | 5.209 |
| ATOM | 1784 | CE2 | TRP | 174 | −32.446 | −9.47 | 5.692 |
| ATOM | 1785 | CE3 | TRP | 174 | −31.16 | −7.801 | 6.883 |
| ATOM | 1786 | CZ2 | TRP | 174 | −33.582 | −8.664 | 5.649 |
| ATOM | 1787 | CZ3 | TRP | 174 | −32.284 | −6.964 | 6.823 |
| ATOM | 1788 | CH2 | TRP | 174 | −33.482 | −7.396 | 6.232 |
| ATOM | 1789 | H | TRP | 174 | −28.007 | −10.121 | 4.03 |
| ATOM | 1790 | HB2 | TRP | 174 | −28.967 | −10.374 | 7.625 |
| ATOM | 1791 | HB1 | TRP | 174 | −28.545 | −11.399 | 6.232 |
| ATOM | 1792 | HD1 | TRP | 174 | −30.729 | −12.159 | 5.119 |
| ATOM | 1793 | HE1 | TRP | 174 | −33.053 | −11.246 | 4.754 |
| ATOM | 1794 | HE3 | TRP | 174 | −30.256 | −7.48 | 7.386 |
| ATOM | 1795 | HZ2 | TRP | 174 | −34.498 | −9.01 | 5.184 |
| ATOM | 1796 | HZ3 | TRP | 174 | −32.227 | −5.965 | 7.243 |
| ATOM | 1797 | HH2 | TRP | 174 | −34.348 | −6.744 | 6.225 |
| ATOM | 1798 | HA | TRP | 174 | −28.402 | −8.336 | 6.234 |
| ATOM | 1799 | N | ALA | 175 | −25.715 | −10.312 | 6.402 |
| ATOM | 1800 | CA | ALA | 175 | −24.449 | −10.352 | 7.134 |
| ATOM | 1801 | C | ALA | 175 | −23.794 | −8.986 | 7.096 |
| ATOM | 1802 | O | ALA | 175 | −23.28 | −8.541 | 8.111 |
| ATOM | 1803 | CB | ALA | 175 | −23.469 | −11.419 | 6.582 |
| ATOM | 1804 | HB3 | ALA | 175 | −23.158 | −11.178 | 5.556 |
| ATOM | 1805 | HB2 | ALA | 175 | −23.95 | −12.409 | 6.583 |
| ATOM | 1806 | HB1 | ALA | 175 | −22.568 | −11.464 | 7.214 |
| ATOM | 1807 | H | ALA | 175 | −25.85 | −10.917 | 5.613 |
| ATOM | 1808 | HA | ALA | 175 | −24.673 | −10.613 | 8.182 |
| ATOM | 1809 | N | ALA | 176 | −23.811 | −8.289 | 5.936 |
| ATOM | 1810 | CA | ALA | 176 | −23.255 | −6.936 | 5.897 |
| ATOM | 1811 | C | ALA | 176 | −23.96 | −6.064 | 6.917 |
| ATOM | 1812 | O | ALA | 176 | −23.315 | −5.275 | 7.591 |
| ATOM | 1813 | CB | ALA | 176 | −23.366 | −6.285 | 4.494 |
| ATOM | 1814 | HB3 | ALA | 176 | −24.414 | −6.081 | 4.232 |
| ATOM | 1815 | HB2 | ALA | 176 | −22.931 | −6.95 | 3.731 |
| ATOM | 1816 | HB1 | ALA | 176 | −22.813 | −5.334 | 4.481 |
| ATOM | 1817 | H | ALA | 176 | −24.218 | −8.677 | 5.105 |
| ATOM | 1818 | HA | ALA | 176 | −22.185 | −7.011 | 6.145 |
| ATOM | 1819 | N | CYS | 177 | −25.298 | −6.198 | 7.059 |
| ATOM | 1820 | CA | CYS | 177 | −25.988 | −5.451 | 8.108 |
| ATOM | 1821 | C | CYS | 177 | −25.452 | −5.867 | 9.459 |
| ATOM | 1822 | O | CYS | 177 | −25.147 | −5.009 | 10.274 |
| ATOM | 1823 | CB | CYS | 177 | −27.525 | −5.666 | 8.127 |
| ATOM | 1824 | SG | CYS | 177 | −28.33 | −5.095 | 6.592 |
| ATOM | 1825 | HG | CYS | 177 | −27.911 | −5.725 | 5.931 |
| ATOM | 1826 | HB2 | CYS | 177 | −27.792 | −6.718 | 8.294 |
| ATOM | 1827 | HB1 | CYS | 177 | −27.934 | −5.084 | 8.965 |
| ATOM | 1828 | H | CYS | 177 | −25.816 | −6.839 | 6.488 |
| ATOM | 1829 | HA | CYS | 177 | −25.8 | −4.377 | 7.971 |
| ATOM | 1830 | LPG2 | CYS | 177 | −28.179 | −4.447 | 6.455 |
| ATOM | 1831 | LPG1 | CYS | 177 | −29.005 | −5.169 | 6.619 |
| ATOM | 1832 | N | THR | 178 | −25.343 | −7.188 | 9.722 |
| ATOM | 1833 | CA | THR | 178 | −24.892 | −7.632 | 11.037 |
| ATOM | 1834 | C | THR | 178 | −23.567 | −7.001 | 11.385 |
| ATOM | 1835 | O | THR | 178 | −23.438 | −6.49 | 12.488 |
| ATOM | 1836 | CB | THR | 178 | −24.759 | −9.178 | 11.106 |
| ATOM | 1837 | OG1 | THR | 178 | −26.025 | −9.737 | 10.713 |
| ATOM | 1838 | CG2 | THR | 178 | −24.376 | −9.656 | 12.534 |
| ATOM | 1839 | HB | THR | 178 | −23.976 | −9.501 | 10.4 |
| ATOM | 1840 | HG22 | THR | 178 | −24.307 | −10.754 | 12.559 |
| ATOM | 1841 | HG21 | THR | 178 | −25.14 | −9.337 | 13.258 |
| ATOM | 1842 | HG23 | THR | 178 | −23.402 | −9.245 | 12.841 |
| ATOM | 1843 | HG1 | THR | 178 | −26.024 | −10.689 | 10.69 |
| ATOM | 1844 | H | THR | 178 | −25.597 | −7.88 | 9.042 |
| ATOM | 1845 | HA | THR | 178 | −25.652 | −7.321 | 11.773 |
| ATOM | 1846 | N | VAL | 179 | −22.562 | −7.022 | 10.479 |
| ATOM | 1847 | CA | VAL | 179 | −21.272 | −6.459 | 10.869 |
| ATOM | 1848 | C | VAL | 179 | −21.463 | −4.986 | 11.143 |
| ATOM | 1849 | O | VAL | 179 | −20.94 | −4.525 | 12.143 |
| ATOM | 1850 | CB | VAL | 179 | −20.049 | −6.723 | 9.939 |
| ATOM | 1851 | CG1 | VAL | 179 | −19.871 | −8.242 | 9.658 |
| ATOM | 1852 | CG2 | VAL | 179 | −20.106 | −5.917 | 8.617 |
| ATOM | 1853 | HB | VAL | 179 | −19.146 | −6.379 | 10.477 |
| ATOM | 1854 | HG23 | VAL | 179 | −21.01 | −6.183 | 8.058 |
| ATOM | 1855 | HG22 | VAL | 179 | −19.229 | −6.143 | 7.991 |
| ATOM | 1856 | HG21 | VAL | 179 | −20.113 | −4.835 | 8.814 |
| ATOM | 1857 | HG13 | VAL | 179 | −19.831 | −8.807 | 10.602 |
| ATOM | 1858 | HG12 | VAL | 179 | −18.932 | −8.42 | 9.111 |
| ATOM | 1859 | HG11 | VAL | 179 | −20.698 | −8.635 | 9.049 |
| ATOM | 1860 | H | VAL | 179 | −22.693 | −7.396 | 9.557 |
| ATOM | 1861 | HA | VAL | 179 | −21.01 | −6.94 | 11.827 |
| ATOM | 1862 | N | SER | 180 | −22.212 | −4.218 | 10.318 |
| ATOM | 1863 | CA | SER | 180 | −22.416 | −2.81 | 10.664 |
| ATOM | 1864 | C | SER | 180 | −22.952 | −2.71 | 12.075 |
| ATOM | 1865 | O | SER | 180 | −22.488 | −1.883 | 12.845 |
| ATOM | 1866 | CB | SER | 180 | −23.395 | −2.068 | 9.722 |
| ATOM | 1867 | OG | SER | 180 | −23.515 | −0.705 | 10.172 |
| ATOM | 1868 | HB2 | SER | 180 | −22.988 | −2.096 | 8.7 |
| ATOM | 1869 | HB1 | SER | 180 | −24.379 | −2.566 | 9.73 |
| ATOM | 1870 | HG | SER | 180 | −24.116 | −0.186 | 9.648 |
| ATOM | 1871 | H | SER | 180 | −22.651 | −4.595 | 9.497 |
| ATOM | 1872 | HA | SER | 180 | −21.444 | −2.295 | 10.603 |
| ATOM | 1873 | N | GLY | 181 | −23.93 | −3.57 | 12.439 |
| ATOM | 1874 | CA | GLY | 181 | −24.406 | −3.571 | 13.818 |
| ATOM | 1875 | C | GLY | 181 | −23.257 | −3.793 | 14.772 |
| ATOM | 1876 | O | GLY | 181 | −23.18 | −3.098 | 15.771 |
| ATOM | 1877 | H | GLY | 181 | −24.318 | −4.234 | 11.79 |
| ATOM | 1878 | HA1 | GLY | 181 | −24.896 | −2.608 | 14.034 |
| ATOM | 1879 | HA2 | GLY | 181 | −25.14 | −4.376 | 13.985 |
| ATOM | 1880 | N | ILE | 182 | −22.349 | −4.755 | 14.494 |
| ATOM | 1881 | CA | ILE | 182 | −21.218 | −4.97 | 15.4 |
| ATOM | 1882 | C | ILE | 182 | −20.38 | −3.71 | 15.445 |
| ATOM | 1883 | O | ILE | 182 | −20.051 | −3.257 | 16.53 |
| ATOM | 1884 | CB | ILE | 182 | −20.341 | −6.207 | 15.027 |
| ATOM | 1885 | CG1 | ILE | 182 | −21.175 | −7.52 | 15.168 |
| ATOM | 1886 | CG2 | ILE | 182 | −19.058 | −6.254 | 15.909 |
| ATOM | 1887 | CD1 | ILE | 182 | −20.462 | −8.788 | 14.623 |
| ATOM | 1888 | HB | ILE | 182 | −20.017 | −6.096 | 13.98 |
| ATOM | 1889 | HG23 | ILE | 182 | −18.452 | −5.345 | 15.78 |
| ATOM | 1890 | HG22 | ILE | 182 | −18.412 | −7.1 | 15.636 |
| ATOM | 1891 | HG21 | ILE | 182 | −19.328 | −6.345 | 16.972 |
| ATOM | 1892 | HG12 | ILE | 182 | −22.119 | −7.423 | 14.61 |
| ATOM | 1893 | HG11 | ILE | 182 | −21.434 | −7.683 | 16.227 |
| ATOM | 1894 | HD13 | ILE | 182 | −20.113 | −8.623 | 13.592 |
| ATOM | 1895 | HD12 | ILE | 182 | −21.164 | −9.637 | 14.618 |
| ATOM | 1896 | HD11 | ILE | 182 | −19.604 | −9.072 | 15.248 |
| ATOM | 1897 | H | ILE | 182 | −22.425 | −5.293 | 13.652 |
| ATOM | 1898 | HA | ILE | 182 | −21.616 | −5.159 | 16.408 |
| ATOM | 1899 | N | LEU | 183 | −20.018 | −3.135 | 14.275 |
| ATOM | 1900 | CA | LEU | 183 | −19.164 | −1.947 | 14.264 |
| ATOM | 1901 | C | LEU | 183 | −19.724 | −0.908 | 15.208 |
| ATOM | 1902 | O | LEU | 183 | −18.966 | −0.39 | 16.012 |
| ATOM | 1903 | CB | LEU | 183 | −19.021 | −1.31 | 12.848 |
| ATOM | 1904 | CG | LEU | 183 | −17.877 | −1.904 | 11.968 |
| ATOM | 1905 | CD1 | LEU | 183 | −17.905 | −3.452 | 11.838 |
| ATOM | 1906 | CD2 | LEU | 183 | −17.939 | −1.282 | 10.545 |
| ATOM | 1907 | HB2 | LEU | 183 | −19.985 | −1.375 | 12.326 |
| ATOM | 1908 | HB1 | LEU | 183 | −18.792 | −0.237 | 12.967 |
| ATOM | 1909 | HG | LEU | 183 | −16.914 | −1.626 | 12.435 |
| ATOM | 1910 | HD13 | LEU | 183 | −17.889 | −3.948 | 12.82 |
| ATOM | 1911 | HD12 | LEU | 183 | −17.024 | −3.796 | 11.274 |
| ATOM | 1912 | HD11 | LEU | 183 | −18.799 | −3.769 | 11.287 |
| ATOM | 1913 | HD23 | LEU | 183 | −18.847 | −1.62 | 10.024 |
| ATOM | 1914 | HD22 | LEU | 183 | −17.065 | −1.583 | 9.947 |
| ATOM | 1915 | HD21 | LEU | 183 | −17.966 | −0.185 | 10.6 |
| ATOM | 1916 | H | LEU | 183 | −20.328 | −3.523 | 13.406 |
| ATOM | 1917 | HA | LEU | 183 | −18.164 | −2.225 | 14.637 |
| ATOM | 1918 | N | PHE | 184 | −21.032 | −0.573 | 15.14 |
| ATOM | 1919 | CA | PHE | 184 | −21.548 | 0.436 | 16.065 |
| ATOM | 1920 | C | PHE | 184 | −21.742 | −0.126 | 17.451 |
| ATOM | 1921 | O | PHE | 184 | −21.452 | 0.591 | 18.392 |
| ATOM | 1922 | CB | PHE | 184 | −22.883 | 1.074 | 15.62 |
| ATOM | 1923 | CG | PHE | 184 | −23.357 | 2.027 | 16.732 |
| ATOM | 1924 | CD1 | PHE | 184 | −22.726 | 3.264 | 16.916 |
| ATOM | 1925 | CD2 | PHE | 184 | −24.407 | 1.663 | 17.582 |
| ATOM | 1926 | CE1 | PHE | 184 | −23.122 | 4.117 | 17.95 |
| ATOM | 1927 | CE2 | PHE | 184 | −24.779 | 2.497 | 18.64 |
| ATOM | 1928 | CZ | PHE | 184 | −24.13 | 3.72 | 18.833 |
| ATOM | 1929 | HB2 | PHE | 184 | −22.713 | 1.626 | 14.684 |
| ATOM | 1930 | HB1 | PHE | 184 | −23.628 | 0.285 | 15.432 |
| ATOM | 1931 | HD1 | PHE | 184 | −21.923 | 3.569 | 16.253 |
| ATOM | 1932 | HD2 | PHE | 184 | −24.936 | 0.727 | 17.433 |
| ATOM | 1933 | HE1 | PHE | 184 | −22.645 | 5.085 | 18.068 |
| ATOM | 1934 | HE2 | PHE | 184 | −25.572 | 2.192 | 19.312 |
| ATOM | 1935 | HZ | PHE | 184 | −24.406 | 4.357 | 19.666 |
| ATOM | 1936 | H | PHE | 184 | −21.652 | −1.018 | 14.487 |
| ATOM | 1937 | HA | PHE | 184 | −20.813 | 1.257 | 16.136 |
| ATOM | 1938 | N | ILE | 185 | −22.226 | −1.371 | 17.65 |
| ATOM | 1939 | CA | ILE | 185 | −22.337 | −1.88 | 19.022 |
| ATOM | 1940 | C | ILE | 185 | −21 | −1.669 | 19.701 |
| ATOM | 1941 | O | ILE | 185 | −20.977 | −1.304 | 20.866 |
| ATOM | 1942 | CB | ILE | 185 | −22.8 | −3.366 | 19.082 |
| ATOM | 1943 | CG1 | ILE | 185 | −24.31 | −3.47 | 18.696 |
| ATOM | 1944 | CG2 | ILE | 185 | −22.537 | −3.982 | 20.487 |
| ATOM | 1945 | CD1 | ILE | 185 | −24.738 | −4.896 | 18.256 |
| ATOM | 1946 | HB | ILE | 185 | −22.187 | −3.924 | 18.359 |
| ATOM | 1947 | HG23 | ILE | 185 | −21.461 | −3.997 | 20.719 |
| ATOM | 1948 | HG22 | ILE | 185 | −22.892 | −5.021 | 20.542 |
| ATOM | 1949 | HG21 | ILE | 185 | −23.054 | −3.395 | 21.262 |
| ATOM | 1950 | HG12 | ILE | 185 | −24.539 | −2.79 | 17.861 |
| ATOM | 1951 | HG11 | ILE | 185 | −24.93 | −3.149 | 19.549 |
| ATOM | 1952 | HD13 | ILE | 185 | −24.128 | −5.235 | 17.404 |
| ATOM | 1953 | HD12 | ILE | 185 | −25.794 | −4.888 | 17.941 |
| ATOM | 1954 | HD11 | ILE | 185 | −24.636 | −5.623 | 19.074 |
| ATOM | 1955 | H | ILE | 185 | −22.463 | −1.956 | 16.873 |
| ATOM | 1956 | HA | ILE | 185 | −23.076 | −1.264 | 19.562 |
| ATOM | 1957 | N | ILE | 186 | −19.87 | −1.848 | 18.981 |
| ATOM | 1958 | CA | ILE | 186 | −18.588 | −1.405 | 19.527 |
| ATOM | 1959 | C | ILE | 186 | −18.595 | 0.096 | 19.307 |
| ATOM | 1960 | O | ILE | 186 | −17.913 | 0.572 | 18.413 |
| ATOM | 1961 | CB | ILE | 186 | −17.396 | −2.141 | 18.838 |
| ATOM | 1962 | CG1 | ILE | 186 | −17.556 | −3.688 | 18.993 |
| ATOM | 1963 | CG2 | ILE | 186 | −16.039 | −1.657 | 19.429 |
| ATOM | 1964 | CD1 | ILE | 186 | −16.494 | −4.505 | 18.208 |
| ATOM | 1965 | HB | ILE | 186 | −17.419 | −1.895 | 17.762 |
| ATOM | 1966 | HG23 | ILE | 186 | −15.954 | −0.561 | 19.411 |
| ATOM | 1967 | HG22 | ILE | 186 | −15.193 | −2.054 | 18.851 |
| ATOM | 1968 | HG21 | ILE | 186 | −15.939 | −1.988 | 20.474 |
| ATOM | 1969 | HG12 | ILE | 186 | −18.54 | −4.005 | 18.616 |
| ATOM | 1970 | HG11 | ILE | 186 | −17.508 | −3.961 | 20.059 |
| ATOM | 1971 | HD13 | ILE | 186 | −16.464 | −4.184 | 17.155 |
| ATOM | 1972 | HD12 | ILE | 186 | −16.748 | −5.576 | 18.237 |
| ATOM | 1973 | HD11 | ILE | 186 | −15.492 | −4.388 | 18.646 |
| ATOM | 1974 | H | ILE | 186 | −19.907 | −2.148 | 18.023 |
| ATOM | 1975 | HA | ILE | 186 | −18.523 | −1.615 | 20.607 |
| ATOM | 1976 | N | TYR | 187 | −19.371 | 0.885 | 20.09 |
| ATOM | 1977 | CA | TYR | 187 | −19.538 | 2.31 | 19.778 |
| ATOM | 1978 | C | TYR | 187 | −18.314 | 3.137 | 20.117 |
| ATOM | 1979 | O | TYR | 187 | −18.425 | 4.124 | 20.826 |
| ATOM | 1980 | CB | TYR | 187 | −20.866 | 2.933 | 20.32 |
| ATOM | 1981 | CG | TYR | 187 | −21.035 | 2.906 | 21.848 |
| ATOM | 1982 | CD1 | TYR | 187 | −20.654 | 3.999 | 22.638 |
| ATOM | 1983 | CD2 | TYR | 187 | −21.607 | 1.793 | 22.476 |
| ATOM | 1984 | CE1 | TYR | 187 | −20.744 | 3.94 | 24.031 |
| ATOM | 1985 | CE2 | TYR | 187 | −21.695 | 1.73 | 23.869 |
| ATOM | 1986 | CZ | TYR | 187 | −21.257 | 2.797 | 24.656 |
| ATOM | 1987 | OH | TYR | 187 | −21.342 | 2.702 | 26.049 |
| ATOM | 1988 | HB1 | TYR | 187 | −20.933 | 3.973 | 19.97 |
| ATOM | 1989 | HB2 | TYR | 187 | −21.735 | 2.413 | 19.895 |
| ATOM | 1990 | HD1 | TYR | 187 | −20.289 | 4.91 | 22.177 |
| ATOM | 1991 | HD2 | TYR | 187 | −21.995 | 0.969 | 21.886 |
| ATOM | 1992 | HE1 | TYR | 187 | −20.414 | 4.79 | 24.62 |
| ATOM | 1993 | HE2 | TYR | 187 | −22.108 | 0.847 | 24.347 |
| ATOM | 1994 | HH | TYR | 187 | −21.028 | 3.469 | 26.513 |
| ATOM | 1995 | H | TYR | 187 | −19.879 | 0.491 | 20.854 |
| ATOM | 1996 | HA | TYR | 187 | −19.622 | 2.408 | 18.681 |
| ATOM | 1997 | N | SER | 188 | −17.116 | 2.807 | 19.585 |
| ATOM | 1998 | CA | SER | 188 | −15.989 | 3.731 | 19.695 |
| ATOM | 1999 | C | SER | 188 | −16.268 | 4.762 | 18.624 |
| ATOM | 2000 | O | SER | 188 | −15.666 | 4.693 | 17.564 |
| ATOM | 2001 | CB | SER | 188 | −14.641 | 2.998 | 19.483 |
| ATOM | 2002 | OG | SER | 188 | −13.573 | 3.96 | 19.551 |
| ATOM | 2003 | HB2 | SER | 188 | −14.522 | 2.237 | 20.273 |
| ATOM | 2004 | HB1 | SER | 188 | −14.655 | 2.485 | 18.508 |
| ATOM | 2005 | HG | SER | 188 | −12.714 | 3.57 | 19.428 |
| ATOM | 2006 | H | SER | 188 | −17.019 | 2.013 | 18.985 |
| ATOM | 2007 | HA | SER | 188 | −15.942 | 4.211 | 20.687 |
| ATOM | 2008 | N | ASP | 189 | −17.221 | 5.687 | 18.894 |
| ATOM | 2009 | CA | ASP | 189 | −17.799 | 6.518 | 17.836 |
| ATOM | 2010 | C | ASP | 189 | −16.795 | 6.904 | 16.774 |
| ATOM | 2011 | O | ASP | 189 | −17 | 6.566 | 15.622 |
| ATOM | 2012 | CB | ASP | 189 | −18.507 | 7.791 | 18.383 |
| ATOM | 2013 | CG | ASP | 189 | −19.551 | 8.271 | 17.408 |
| ATOM | 2014 | OD1 | ASP | 189 | −19.188 | 9.049 | 16.489 |
| ATOM | 2015 | OD2 | ASP | 189 | −20.739 | 7.87 | 17.555 |
| ATOM | 2016 | HB2 | ASP | 189 | −19.019 | 7.574 | 19.33 |
| ATOM | 2017 | HB1 | ASP | 189 | −17.785 | 8.596 | 18.583 |
| ATOM | 2018 | H | ASP | 189 | −17.659 | 5.704 | 19.797 |
| ATOM | 2019 | HA | ASP | 189 | −18.578 | 5.893 | 17.365 |
| ATOM | 2020 | N | SER | 190 | −15.69 | 7.603 | 17.11 |
| ATOM | 2021 | CA | SER | 190 | −14.79 | 8.07 | 16.057 |
| ATOM | 2022 | C | SER | 190 | −14.255 | 6.943 | 15.2 |
| ATOM | 2023 | O | SER | 190 | −14.316 | 7.05 | 13.985 |
| ATOM | 2024 | CB | SER | 190 | −13.597 | 8.862 | 16.647 |
| ATOM | 2025 | OG | SER | 190 | −14.117 | 9.944 | 17.44 |
| ATOM | 2026 | HB2 | SER | 190 | −12.985 | 8.194 | 17.276 |
| ATOM | 2027 | HB1 | SER | 190 | −12.97 | 9.252 | 15.826 |
| ATOM | 2028 | HG | SER | 190 | −13.435 | 10.483 | 17.829 |
| ATOM | 2029 | H | SER | 190 | −15.499 | 7.848 | 18.062 |
| ATOM | 2030 | HA | SER | 190 | −15.358 | 8.762 | 15.414 |
| ATOM | 2031 | N | SER | 191 | −13.71 | 5.852 | 15.782 |
| ATOM | 2032 | CA | SER | 191 | −13.154 | 4.803 | 14.928 |
| ATOM | 2033 | C | SER | 191 | −14.29 | 4.072 | 14.255 |
| ATOM | 2034 | O | SER | 191 | −14.243 | 3.916 | 13.045 |
| ATOM | 2035 | CB | SER | 191 | −12.263 | 3.79 | 15.689 |
| ATOM | 2036 | OG | SER | 191 | −13.054 | 3.048 | 16.629 |
| ATOM | 2037 | H | SER | 191 | −13.715 | 5.724 | 16.776 |
| ATOM | 2038 | HB2 | SER | 191 | −11.459 | 4.336 | 16.211 |
| ATOM | 2039 | HB1 | SER | 191 | −11.807 | 3.098 | 14.959 |
| ATOM | 2040 | HG | SER | 191 | −12.546 | 2.387 | 17.089 |
| ATOM | 2041 | HA | SER | 191 | −12.516 | 5.273 | 14.161 |
| ATOM | 2042 | N | ALA | 192 | −15.314 | 3.635 | 15.026 |
| ATOM | 2043 | CA | ALA | 192 | −16.475 | 2.98 | 14.421 |
| ATOM | 2044 | C | ALA | 192 | −16.946 | 3.782 | 13.227 |
| ATOM | 2045 | O | ALA | 192 | −17.179 | 3.216 | 12.17 |
| ATOM | 2046 | CB | ALA | 192 | −17.642 | 2.838 | 15.435 |
| ATOM | 2047 | HB3 | ALA | 192 | −17.954 | 3.825 | 15.809 |
| ATOM | 2048 | HB2 | ALA | 192 | −17.328 | 2.223 | 16.292 |
| ATOM | 2049 | HB1 | ALA | 192 | −18.507 | 2.353 | 14.955 |
| ATOM | 2050 | H | ALA | 192 | −15.294 | 3.768 | 16.021 |
| ATOM | 2051 | HA | ALA | 192 | −16.187 | 1.966 | 14.101 |
| ATOM | 2052 | N | VAL | 193 | −17.073 | 5.117 | 13.39 |
| ATOM | 2053 | CA | VAL | 193 | −17.467 | 5.976 | 12.277 |
| ATOM | 2054 | C | VAL | 193 | −16.458 | 5.865 | 11.16 |
| ATOM | 2055 | O | VAL | 193 | −16.858 | 5.633 | 10.032 |
| ATOM | 2056 | CB | VAL | 193 | −17.622 | 7.469 | 12.707 |
| ATOM | 2057 | CG1 | VAL | 193 | −17.508 | 8.446 | 11.504 |
| ATOM | 2058 | CG2 | VAL | 193 | −18.971 | 7.674 | 13.455 |
| ATOM | 2059 | H | VAL | 193 | −16.865 | 5.538 | 14.274 |
| ATOM | 2060 | HB | VAL | 193 | −16.797 | 7.746 | 13.384 |
| ATOM | 2061 | HG13 | VAL | 193 | −16.495 | 8.449 | 11.072 |
| ATOM | 2062 | HG12 | VAL | 193 | −17.728 | 9.47 | 11.834 |
| ATOM | 2063 | HG11 | VAL | 193 | −18.219 | 8.165 | 10.716 |
| ATOM | 2064 | HG23 | VAL | 193 | −19.819 | 7.46 | 12.787 |
| ATOM | 2065 | HG22 | VAL | 193 | −19.06 | 8.711 | 13.815 |
| ATOM | 2066 | HG21 | VAL | 193 | −19.055 | 7.002 | 14.321 |
| ATOM | 2067 | HA | VAL | 193 | −18.437 | 5.625 | 11.889 |
| ATOM | 2068 | N | ILE | 194 | −15.149 | 6.05 | 11.427 |
| ATOM | 2069 | CA | ILE | 194 | −14.192 | 6.052 | 10.32 |
| ATOM | 2070 | C | ILE | 194 | −14.301 | 4.744 | 9.567 |
| ATOM | 2071 | O | ILE | 194 | −14.502 | 4.773 | 8.361 |
| ATOM | 2072 | CB | ILE | 194 | −12.732 | 6.335 | 10.791 |
| ATOM | 2073 | CG1 | ILE | 194 | −12.598 | 7.824 | 11.243 |
| ATOM | 2074 | CG2 | ILE | 194 | −11.708 | 5.995 | 9.668 |
| ATOM | 2075 | CD1 | ILE | 194 | −11.358 | 8.092 | 12.138 |
| ATOM | 2076 | H | ILE | 194 | −14.837 | 6.2 | 12.369 |
| ATOM | 2077 | HB | ILE | 194 | −12.522 | 5.672 | 11.646 |
| ATOM | 2078 | HG12 | ILE | 194 | −13.484 | 8.127 | 11.822 |
| ATOM | 2079 | HG11 | ILE | 194 | −12.555 | 8.476 | 10.355 |
| ATOM | 2080 | HG23 | ILE | 194 | −11.742 | 4.926 | 9.406 |
| ATOM | 2081 | HG22 | ILE | 194 | −10.679 | 6.216 | 9.984 |
| ATOM | 2082 | HG21 | ILE | 194 | −11.929 | 6.581 | 8.763 |
| ATOM | 2083 | HD13 | ILE | 194 | −11.385 | 7.449 | 13.032 |
| ATOM | 2084 | HD12 | ILE | 194 | −11.358 | 9.143 | 12.47 |
| ATOM | 2085 | HD11 | ILE | 194 | −10.417 | 7.909 | 11.599 |
| ATOM | 2086 | HA | ILE | 194 | −14.475 | 6.857 | 9.62 |
| ATOM | 2087 | N | ILE | 195 | −14.164 | 3.583 | 10.247 |
| ATOM | 2088 | CA | ILE | 195 | −14.233 | 2.32 | 9.511 |
| ATOM | 2089 | C | ILE | 195 | −15.58 | 2.21 | 8.827 |
| ATOM | 2090 | O | ILE | 195 | −15.624 | 1.856 | 7.659 |
| ATOM | 2091 | CB | ILE | 195 | −13.858 | 1.031 | 10.313 |
| ATOM | 2092 | CG1 | ILE | 195 | −13.878 | −0.253 | 9.424 |
| ATOM | 2093 | CG2 | ILE | 195 | −14.774 | 0.786 | 11.544 |
| ATOM | 2094 | CD1 | ILE | 195 | −12.932 | −0.206 | 8.193 |
| ATOM | 2095 | HB | ILE | 195 | −12.831 | 1.167 | 10.695 |
| ATOM | 2096 | HG23 | ILE | 195 | −14.719 | 1.619 | 12.25 |
| ATOM | 2097 | HG22 | ILE | 195 | −14.465 | −0.121 | 12.086 |
| ATOM | 2098 | HG21 | ILE | 195 | −15.818 | 0.659 | 11.235 |
| ATOM | 2099 | HG12 | ILE | 195 | −14.905 | −0.45 | 9.081 |
| ATOM | 2100 | HG11 | ILE | 195 | −13.564 | −1.112 | 10.042 |
| ATOM | 2101 | HD13 | ILE | 195 | −13.295 | 0.496 | 7.431 |
| ATOM | 2102 | HD12 | ILE | 195 | −12.878 | −1.202 | 7.725 |
| ATOM | 2103 | HD11 | ILE | 195 | −11.917 | 0.09 | 8.5 |
| ATOM | 2104 | H | ILE | 195 | −14.011 | 3.588 | 11.237 |
| ATOM | 2105 | HA | ILE | 195 | −13.455 | 2.415 | 8.741 |
| ATOM | 2106 | N | CYS | 196 | −16.698 | 2.52 | 9.522 |
| ATOM | 2107 | CA | CYS | 196 | −18.014 | 2.392 | 8.894 |
| ATOM | 2108 | C | CYS | 196 | −18.091 | 3.231 | 7.639 |
| ATOM | 2109 | O | CYS | 196 | −18.418 | 2.719 | 6.58 |
| ATOM | 2110 | CB | CYS | 196 | −19.138 | 2.826 | 9.878 |
| ATOM | 2111 | SG | CYS | 196 | −20.788 | 2.833 | 9.103 |
| ATOM | 2112 | HG | CYS | 196 | −20.946 | 1.839 | 9.078 |
| ATOM | 2113 | HB2 | CYS | 196 | −19.155 | 2.167 | 10.761 |
| ATOM | 2114 | HB1 | CYS | 196 | −18.96 | 3.854 | 10.221 |
| ATOM | 2115 | H | CYS | 196 | −16.639 | 2.851 | 10.468 |
| ATOM | 2116 | HA | CYS | 196 | −18.177 | 1.338 | 8.621 |
| ATOM | 2117 | LPG2 | CYS | 196 | −20.778 | 3.106 | 8.481 |
| ATOM | 2118 | LPG1 | CYS | 196 | −21.249 | 3.156 | 9.483 |
| ATOM | 2119 | N | LEU | 197 | −17.795 | 4.541 | 7.738 |
| ATOM | 2120 | CA | LEU | 197 | −17.967 | 5.429 | 6.589 |
| ATOM | 2121 | C | LEU | 197 | −17.136 | 4.938 | 5.424 |
| ATOM | 2122 | O | LEU | 197 | −17.654 | 4.908 | 4.317 |
| ATOM | 2123 | CB | LEU | 197 | −17.64 | 6.907 | 6.963 |
| ATOM | 2124 | CG | LEU | 197 | −18.84 | 7.712 | 7.571 |
| ATOM | 2125 | CD1 | LEU | 197 | −19.684 | 8.38 | 6.449 |
| ATOM | 2126 | CD2 | LEU | 197 | −19.768 | 6.898 | 8.521 |
| ATOM | 2127 | HB2 | LEU | 197 | −16.781 | 6.92 | 7.653 |
| ATOM | 2128 | HB1 | LEU | 197 | −17.302 | 7.431 | 6.056 |
| ATOM | 2129 | HG | LEU | 197 | −18.42 | 8.535 | 8.175 |
| ATOM | 2130 | HD13 | LEU | 197 | −19.084 | 9.134 | 5.916 |
| ATOM | 2131 | HD12 | LEU | 197 | −20.565 | 8.891 | 6.869 |
| ATOM | 2132 | HD11 | LEU | 197 | −20.016 | 7.626 | 5.721 |
| ATOM | 2133 | HD23 | LEU | 197 | −20.348 | 6.136 | 7.982 |
| ATOM | 2134 | HD22 | LEU | 197 | −20.49 | 7.571 | 9.01 |
| ATOM | 2135 | HD21 | LEU | 197 | −19.192 | 6.401 | 9.312 |
| ATOM | 2136 | H | LEU | 197 | −17.445 | 4.905 | 8.602 |
| ATOM | 2137 | HA | LEU | 197 | −19.011 | 5.367 | 6.245 |
| ATOM | 2138 | N | ILE | 198 | −15.856 | 4.543 | 5.609 |
| ATOM | 2139 | CA | ILE | 198 | −15.098 | 4.087 | 4.443 |
| ATOM | 2140 | C | ILE | 198 | −15.623 | 2.762 | 3.926 |
| ATOM | 2141 | O | ILE | 198 | −15.914 | 2.683 | 2.742 |
| ATOM | 2142 | CB | ILE | 198 | −13.542 | 4.083 | 4.587 |
| ATOM | 2143 | CG1 | ILE | 198 | −12.867 | 3.895 | 3.19 |
| ATOM | 2144 | CG2 | ILE | 198 | −13.035 | 3.006 | 5.584 |
| ATOM | 2145 | CD1 | ILE | 198 | −11.317 | 3.981 | 3.226 |
| ATOM | 2146 | HB | ILE | 198 | −13.257 | 5.073 | 4.983 |
| ATOM | 2147 | HG23 | ILE | 198 | −13.614 | 3.055 | 6.51 |
| ATOM | 2148 | HG22 | ILE | 198 | −11.982 | 3.174 | 5.852 |
| ATOM | 2149 | HG21 | ILE | 198 | −13.131 | 1.997 | 5.157 |
| ATOM | 2150 | HG12 | ILE | 198 | −13.219 | 4.683 | 2.505 |
| ATOM | 2151 | HG11 | ILE | 198 | −13.156 | 2.923 | 2.758 |
| ATOM | 2152 | HD13 | ILE | 198 | −10.995 | 4.906 | 3.728 |
| ATOM | 2153 | HD12 | ILE | 198 | −10.919 | 3.985 | 2.199 |
| ATOM | 2154 | HD11 | ILE | 198 | −10.879 | 3.118 | 3.749 |
| ATOM | 2155 | H | ILE | 198 | −15.434 | 4.561 | 6.519 |
| ATOM | 2156 | HA | ILE | 198 | −15.293 | 4.838 | 3.664 |
| ATOM | 2157 | N | THR | 199 | −15.742 | 1.7 | 4.76 |
| ATOM | 2158 | CA | THR | 199 | −16.021 | 0.368 | 4.209 |
| ATOM | 2159 | C | THR | 199 | −17.499 | 0.072 | 4.087 |
| ATOM | 2160 | O | THR | 199 | −17.885 | −0.553 | 3.114 |
| ATOM | 2161 | CB | THR | 199 | −15.268 | −0.762 | 4.969 |
| ATOM | 2162 | OG1 | THR | 199 | −15.136 | −1.896 | 4.089 |
| ATOM | 2163 | CG2 | THR | 199 | −15.99 | −1.217 | 6.264 |
| ATOM | 2164 | HB | THR | 199 | −14.259 | −0.387 | 5.214 |
| ATOM | 2165 | HG22 | THR | 199 | −15.364 | −1.929 | 6.823 |
| ATOM | 2166 | HG21 | THR | 199 | −16.925 | −1.723 | 5.993 |
| ATOM | 2167 | HG23 | THR | 199 | −16.228 | −0.372 | 6.919 |
| ATOM | 2168 | HG1 | THR | 199 | −14.641 | −2.615 | 4.471 |
| ATOM | 2169 | H | THR | 199 | −15.616 | 1.801 | 5.751 |
| ATOM | 2170 | HA | THR | 199 | −15.604 | 0.325 | 3.19 |
| ATOM | 2171 | N | MET | 200 | −18.356 | 0.496 | 5.044 |
| ATOM | 2172 | CA | MET | 200 | −19.789 | 0.222 | 4.919 |
| ATOM | 2173 | C | MET | 200 | −20.463 | 1.205 | 3.981 |
| ATOM | 2174 | O | MET | 200 | −21.63 | 0.981 | 3.697 |
| ATOM | 2175 | CB | MET | 200 | −20.496 | 0.276 | 6.304 |
| ATOM | 2176 | CG | MET | 200 | −19.957 | −0.757 | 7.335 |
| ATOM | 2177 | SD | MET | 200 | −20.639 | −2.432 | 7.087 |
| ATOM | 2178 | CE | MET | 200 | −19.317 | −3.359 | 6.254 |
| ATOM | 2179 | HB2 | MET | 200 | −20.356 | 1.291 | 6.698 |
| ATOM | 2180 | HB1 | MET | 200 | −21.58 | 0.138 | 6.194 |
| ATOM | 2181 | HG2 | MET | 200 | −20.314 | −0.465 | 8.334 |
| ATOM | 2182 | HG1 | MET | 200 | −18.859 | −0.788 | 7.36 |
| ATOM | 2183 | HE3 | MET | 200 | −19.013 | −2.859 | 5.324 |
| ATOM | 2184 | HE2 | MET | 200 | −19.696 | −4.362 | 6.008 |
| ATOM | 2185 | HE1 | MET | 200 | −18.455 | −3.456 | 6.93 |
| ATOM | 2186 | H | MET | 200 | −18.039 | 1.061 | 5.809 |
| ATOM | 2187 | HA | MET | 200 | −19.958 | −0.784 | 4.502 |
| ATOM | 2188 | LPD2 | MET | 200 | −21.196 | −2.408 | 6.698 |
| ATOM | 2189 | LPD1 | MET | 200 | −20.792 | −2.718 | 7.683 |
| ATOM | 2190 | N | PHE | 201 | −19.8 | 2.285 | 3.494 |
| ATOM | 2191 | CA | PHE | 201 | −20.487 | 3.26 | 2.641 |
| ATOM | 2192 | C | PHE | 201 | −19.601 | 3.665 | 1.475 |
| ATOM | 2193 | O | PHE | 201 | −19.945 | 3.344 | 0.348 |
| ATOM | 2194 | CB | PHE | 201 | −20.956 | 4.44 | 3.542 |
| ATOM | 2195 | CG | PHE | 201 | −21.997 | 5.38 | 2.905 |
| ATOM | 2196 | CD1 | PHE | 201 | −23.266 | 5.537 | 3.477 |
| ATOM | 2197 | CD2 | PHE | 201 | −21.687 | 6.118 | 1.756 |
| ATOM | 2198 | CE1 | PHE | 201 | −24.16 | 6.49 | 2.979 |
| ATOM | 2199 | CE2 | PHE | 201 | −22.588 | 7.052 | 1.237 |
| ATOM | 2200 | CZ | PHE | 201 | −23.818 | 7.26 | 1.865 |
| ATOM | 2201 | H | PHE | 201 | −18.852 | 2.481 | 3.748 |
| ATOM | 2202 | HB2 | PHE | 201 | −20.107 | 5.063 | 3.848 |
| ATOM | 2203 | HB1 | PHE | 201 | −21.377 | 4.001 | 4.46 |
| ATOM | 2204 | HD1 | PHE | 201 | −23.565 | 4.924 | 4.321 |
| ATOM | 2205 | HD2 | PHE | 201 | −20.739 | 5.977 | 1.257 |
| ATOM | 2206 | HE1 | PHE | 201 | −25.123 | 6.64 | 3.454 |
| ATOM | 2207 | HE2 | PHE | 201 | −22.331 | 7.62 | 0.348 |
| ATOM | 2208 | HZ | PHE | 201 | −24.504 | 8.014 | 1.49 |
| ATOM | 2209 | HA | PHE | 201 | −21.4 | 2.824 | 2.199 |
| ATOM | 2210 | N | PHE | 202 | −18.462 | 4.371 | 1.677 |
| ATOM | 2211 | CA | PHE | 202 | −17.729 | 4.896 | 0.522 |
| ATOM | 2212 | C | PHE | 202 | −17.356 | 3.784 | −0.433 |
| ATOM | 2213 | O | PHE | 202 | −17.573 | 3.933 | −1.626 |
| ATOM | 2214 | CB | PHE | 202 | −16.426 | 5.663 | 0.89 |
| ATOM | 2215 | CG | PHE | 202 | −16.566 | 6.82 | 1.898 |
| ATOM | 2216 | CD1 | PHE | 202 | −15.409 | 7.313 | 2.514 |
| ATOM | 2217 | CD2 | PHE | 202 | −17.794 | 7.409 | 2.226 |
| ATOM | 2218 | CE1 | PHE | 202 | −15.478 | 8.33 | 3.471 |
| ATOM | 2219 | CE2 | PHE | 202 | −17.868 | 8.443 | 3.163 |
| ATOM | 2220 | CZ | PHE | 202 | −16.71 | 8.904 | 3.792 |
| ATOM | 2221 | HB2 | PHE | 202 | −15.707 | 4.935 | 1.292 |
| ATOM | 2222 | HB1 | PHE | 202 | −15.991 | 6.088 | −0.029 |
| ATOM | 2223 | HD1 | PHE | 202 | −14.438 | 6.907 | 2.249 |
| ATOM | 2224 | HD2 | PHE | 202 | −18.71 | 7.082 | 1.758 |
| ATOM | 2225 | HE1 | PHE | 202 | −14.574 | 8.677 | 3.962 |
| ATOM | 2226 | HE2 | PHE | 202 | −18.829 | 8.889 | 3.399 |
| ATOM | 2227 | HZ | PHE | 202 | −16.766 | 9.704 | 4.524 |
| ATOM | 2228 | H | PHE | 202 | −18.122 | 4.581 | 2.597 |
| ATOM | 2229 | HA | PHE | 202 | −18.393 | 5.584 | −0.028 |
| ATOM | 2230 | N | THR | 203 | −16.788 | 2.661 | 0.059 |
| ATOM | 2231 | CA | THR | 203 | −16.398 | 1.592 | −0.86 |
| ATOM | 2232 | C | THR | 203 | −17.57 | 1.138 | −1.701 |
| ATOM | 2233 | O | THR | 203 | −17.376 | 0.879 | −2.878 |
| ATOM | 2234 | CB | THR | 203 | −15.785 | 0.388 | −0.093 |
| ATOM | 2235 | OG1 | THR | 203 | −14.645 | 0.887 | 0.631 |
| ATOM | 2236 | CG2 | THR | 203 | −15.336 | −0.744 | −1.058 |
| ATOM | 2237 | HB | THR | 203 | −16.54 | 0 | 0.613 |
| ATOM | 2238 | HG22 | THR | 203 | −14.897 | −1.575 | −0.485 |
| ATOM | 2239 | HG21 | THR | 203 | −14.578 | −0.362 | −1.758 |
| ATOM | 2240 | HG23 | THR | 203 | −16.181 | −1.142 | −1.639 |
| ATOM | 2241 | HG1 | THR | 203 | −14.2 | 0.216 | 1.139 |
| ATOM | 2242 | H | THR | 203 | −16.607 | 2.553 | 1.041 |
| ATOM | 2243 | HA | THR | 203 | −15.617 | 1.991 | −1.527 |
| ATOM | 2244 | N | MET | 204 | −18.795 | 1.03 | −1.141 |
| ATOM | 2245 | CA | MET | 204 | −19.923 | 0.579 | −1.958 |
| ATOM | 2246 | C | MET | 204 | −20.197 | 1.594 | −3.047 |
| ATOM | 2247 | O | MET | 204 | −20.384 | 1.199 | −4.188 |
| ATOM | 2248 | CB | MET | 204 | −21.234 | 0.361 | −1.15 |
| ATOM | 2249 | CG | MET | 204 | −21.256 | −0.958 | −0.332 |
| ATOM | 2250 | SD | MET | 204 | −20.007 | −0.969 | 0.993 |
| ATOM | 2251 | CE | MET | 204 | −18.779 | −2.124 | 0.306 |
| ATOM | 2252 | HB2 | MET | 204 | −21.425 | 1.224 | −0.491 |
| ATOM | 2253 | HB1 | MET | 204 | −22.065 | 0.296 | −1.869 |
| ATOM | 2254 | HG2 | MET | 204 | −21.126 | −1.828 | −0.992 |
| ATOM | 2255 | HG1 | MET | 204 | −22.23 | −1.065 | 0.167 |
| ATOM | 2256 | HE3 | MET | 204 | −18.428 | −1.762 | −0.67 |
| ATOM | 2257 | HE2 | MET | 204 | −19.233 | −3.119 | 0.185 |
| ATOM | 2258 | HE1 | MET | 204 | −17.924 | −2.201 | 0.994 |
| ATOM | 2259 | H | MET | 204 | −18.95 | 1.295 | −0.187 |
| ATOM | 2260 | HA | MET | 204 | −19.658 | −0.372 | −2.448 |
| ATOM | 2261 | LPD2 | MET | 204 | −20.266 | −1.199 | 1.577 |
| ATOM | 2262 | LPD1 | MET | 204 | −19.742 | −0.352 | 1.09 |
| ATOM | 2263 | N | LEU | 205 | −20.237 | 2.907 | −2.726 |
| ATOM | 2264 | CA | LEU | 205 | −20.531 | 3.893 | −3.769 |
| ATOM | 2265 | C | LEU | 205 | −19.397 | 3.899 | −4.77 |
| ATOM | 2266 | O | LEU | 205 | −19.66 | 3.878 | −5.962 |
| ATOM | 2267 | CB | LEU | 205 | −20.744 | 5.34 | −3.229 |
| ATOM | 2268 | CG | LEU | 205 | −22.142 | 5.637 | −2.597 |
| ATOM | 2269 | CD1 | LEU | 205 | −23.291 | 5.669 | −3.645 |
| ATOM | 2270 | CD2 | LEU | 205 | −22.494 | 4.659 | −1.447 |
| ATOM | 2271 | H | LEU | 205 | −20.035 | 3.212 | −1.791 |
| ATOM | 2272 | HB2 | LEU | 205 | −20.613 | 6.049 | −4.063 |
| ATOM | 2273 | HB1 | LEU | 205 | −19.951 | 5.555 | −2.494 |
| ATOM | 2274 | HG | LEU | 205 | −22.092 | 6.652 | −2.161 |
| ATOM | 2275 | HD13 | LEU | 205 | −23.074 | 6.4 | −4.439 |
| ATOM | 2276 | HD12 | LEU | 205 | −24.238 | 5.955 | −3.163 |
| ATOM | 2277 | HD11 | LEU | 205 | −23.439 | 4.686 | −4.108 |
| ATOM | 2278 | HD23 | LEU | 205 | −22.548 | 3.628 | −1.823 |
| ATOM | 2279 | HD22 | LEU | 205 | −23.462 | 4.92 | −0.991 |
| ATOM | 2280 | HD21 | LEU | 205 | −21.727 | 4.712 | −0.665 |
| ATOM | 2281 | HA | LEU | 205 | −21.431 | 3.571 | −4.314 |
| ATOM | 2282 | N | ALA | 206 | −18.124 | 3.932 | −4.321 |
| ATOM | 2283 | CA | ALA | 206 | −17.018 | 3.943 | −5.278 |
| ATOM | 2284 | C | ALA | 206 | −17.061 | 2.705 | −6.147 |
| ATOM | 2285 | O | ALA | 206 | −16.903 | 2.808 | −7.355 |
| ATOM | 2286 | CB | ALA | 206 | −15.649 | 3.996 | −4.552 |
| ATOM | 2287 | HB3 | ALA | 206 | −15.521 | 3.112 | −3.908 |
| ATOM | 2288 | HB2 | ALA | 206 | −15.588 | 4.9 | −3.926 |
| ATOM | 2289 | HB1 | ALA | 206 | −14.829 | 4.019 | −5.286 |
| ATOM | 2290 | H | ALA | 206 | −17.926 | 3.942 | −3.339 |
| ATOM | 2291 | HA | ALA | 206 | −17.112 | 4.838 | −5.916 |
| ATOM | 2292 | N | LEU | 207 | −17.274 | 1.515 | −5.544 |
| ATOM | 2293 | CA | LEU | 207 | −17.317 | 0.292 | −6.343 |
| ATOM | 2294 | C | LEU | 207 | −18.45 | 0.405 | −7.33 |
| ATOM | 2295 | O | LEU | 207 | −18.223 | 0.209 | −8.514 |
| ATOM | 2296 | CB | LEU | 207 | −17.446 | −0.965 | −5.436 |
| ATOM | 2297 | CG | LEU | 207 | −17.456 | −2.33 | −6.197 |
| ATOM | 2298 | CD1 | LEU | 207 | −16.975 | −3.46 | −5.242 |
| ATOM | 2299 | CD2 | LEU | 207 | −18.862 | −2.698 | −6.757 |
| ATOM | 2300 | HB2 | LEU | 207 | −16.571 | −0.936 | −4.765 |
| ATOM | 2301 | HB1 | LEU | 207 | −18.346 | −0.889 | −4.806 |
| ATOM | 2302 | HG | LEU | 207 | −16.743 | −2.285 | −7.038 |
| ATOM | 2303 | HD13 | LEU | 207 | −15.936 | −3.284 | −4.922 |
| ATOM | 2304 | HD12 | LEU | 207 | −17.012 | −4.442 | −5.74 |
| ATOM | 2305 | HD11 | LEU | 207 | −17.611 | −3.495 | −4.346 |
| ATOM | 2306 | HD23 | LEU | 207 | −19.609 | −2.685 | −5.95 |
| ATOM | 2307 | HD22 | LEU | 207 | −18.849 | −3.707 | −7.198 |
| ATOM | 2308 | HD21 | LEU | 207 | −19.192 | −2.009 | −7.543 |
| ATOM | 2309 | H | LEU | 207 | −17.42 | 1.457 | −4.553 |
| ATOM | 2310 | HA | LEU | 207 | −16.369 | 0.213 | −6.903 |
| ATOM | 2311 | N | MET | 208 | −19.684 | 0.718 | −6.878 |
| ATOM | 2312 | CA | MET | 208 | −20.783 | 0.806 | −7.834 |
| ATOM | 2313 | C | MET | 208 | −20.559 | 1.913 | −8.834 |
| ATOM | 2314 | O | MET | 208 | −21.022 | 1.767 | −9.953 |
| ATOM | 2315 | CB | MET | 208 | −22.176 | 0.965 | −7.175 |
| ATOM | 2316 | CG | MET | 208 | −22.606 | −0.351 | −6.478 |
| ATOM | 2317 | SD | MET | 208 | −24.352 | −0.192 | −5.999 |
| ATOM | 2318 | CE | MET | 208 | −24.15 | 0.534 | −4.354 |
| ATOM | 2319 | HB2 | MET | 208 | −22.161 | 1.811 | −6.47 |
| ATOM | 2320 | HB1 | MET | 208 | −22.915 | 1.189 | −7.963 |
| ATOM | 2321 | HG2 | MET | 208 | −21.973 | −0.583 | −5.607 |
| ATOM | 2322 | HG1 | MET | 208 | −22.549 | −1.194 | −7.181 |
| ATOM | 2323 | HE3 | MET | 208 | −23.741 | −0.231 | −3.68 |
| ATOM | 2324 | HE2 | MET | 208 | −25.14 | 0.841 | −4.005 |
| ATOM | 2325 | HE1 | MET | 208 | −23.482 | 1.406 | −4.376 |
| ATOM | 2326 | H | MET | 208 | −19.856 | 0.909 | −5.908 |
| ATOM | 2327 | HA | MET | 208 | −20.801 | −0.13 | −8.412 |
| ATOM | 2328 | LPD2 | MET | 208 | −24.664 | −0.795 | −5.973 |
| ATOM | 2329 | LPD1 | MET | 208 | −24.693 | 0.22 | −6.418 |
| ATOM | 2330 | N | ALA | 209 | −19.863 | 3.019 | −8.499 |
| ATOM | 2331 | CA | ALA | 209 | −19.571 | 4.01 | −9.533 |
| ATOM | 2332 | C | ALA | 209 | −18.69 | 3.365 | −10.581 |
| ATOM | 2333 | O | ALA | 209 | −18.991 | 3.455 | −11.761 |
| ATOM | 2334 | CB | ALA | 209 | −18.873 | 5.267 | −8.952 |
| ATOM | 2335 | HB3 | ALA | 209 | −17.912 | 5.003 | −8.487 |
| ATOM | 2336 | HB2 | ALA | 209 | −19.515 | 5.734 | −8.19 |
| ATOM | 2337 | HB1 | ALA | 209 | −18.685 | 5.998 | −9.754 |
| ATOM | 2338 | H | ALA | 209 | −19.508 | 3.154 | −7.571 |
| ATOM | 2339 | HA | ALA | 209 | −20.518 | 4.332 | −9.999 |
| ATOM | 2340 | N | SER | 210 | −17.59 | 2.696 | −10.17 |
| ATOM | 2341 | CA | SER | 210 | −16.712 | 2.067 | −11.158 |
| ATOM | 2342 | C | SER | 210 | −17.457 | 1.005 | −11.938 |
| ATOM | 2343 | O | SER | 210 | −17.339 | 0.94 | −13.152 |
| ATOM | 2344 | CB | SER | 210 | −15.482 | 1.411 | −10.479 |
| ATOM | 2345 | OG | SER | 210 | −14.612 | 0.875 | −11.491 |
| ATOM | 2346 | HB2 | SER | 210 | −14.945 | 2.173 | −9.888 |
| ATOM | 2347 | HB1 | SER | 210 | −15.818 | 0.61 | −9.798 |
| ATOM | 2348 | HG | SER | 210 | −13.845 | 0.441 | −11.131 |
| ATOM | 2349 | H | SER | 210 | −17.373 | 2.612 | −9.193 |
| ATOM | 2350 | HA | SER | 210 | −16.346 | 2.838 | −11.856 |
| ATOM | 2351 | N | LEU | 211 | −18.241 | 0.147 | −11.251 |
| ATOM | 2352 | CA | LEU | 211 | −18.96 | −0.911 | −11.959 |
| ATOM | 2353 | C | LEU | 211 | −19.959 | −0.255 | −12.883 |
| ATOM | 2354 | O | LEU | 211 | −19.988 | −0.582 | −14.058 |
| ATOM | 2355 | CB | LEU | 211 | −19.605 | −1.888 | −10.937 |
| ATOM | 2356 | CG | LEU | 211 | −20.17 | −3.212 | −11.532 |
| ATOM | 2357 | CD1 | LEU | 211 | −20.435 | −4.211 | −10.37 |
| ATOM | 2358 | CD2 | LEU | 211 | −21.471 | −2.992 | −12.357 |
| ATOM | 2359 | HB2 | LEU | 211 | −18.809 | −2.152 | −10.221 |
| ATOM | 2360 | HB1 | LEU | 211 | −20.401 | −1.373 | −10.384 |
| ATOM | 2361 | HG | LEU | 211 | −19.407 | −3.664 | −12.187 |
| ATOM | 2362 | HD13 | LEU | 211 | −20.821 | −5.158 | −10.766 |
| ATOM | 2363 | HD12 | LEU | 211 | −21.167 | −3.804 | −9.658 |
| ATOM | 2364 | HD11 | LEU | 211 | −19.506 | −4.434 | −9.822 |
| ATOM | 2365 | HD23 | LEU | 211 | −22.195 | −2.383 | −11.802 |
| ATOM | 2366 | HD22 | LEU | 211 | −21.947 | −3.95 | −12.61 |
| ATOM | 2367 | HD21 | LEU | 211 | −21.261 | −2.479 | −13.3 |
| ATOM | 2368 | H | LEU | 211 | −18.35 | 0.241 | −10.258 |
| ATOM | 2369 | HA | LEU | 211 | −18.232 | −1.48 | −12.559 |
| ATOM | 2370 | N | TYR | 212 | −20.778 | 0.695 | −12.386 |
| ATOM | 2371 | CA | TYR | 212 | −21.713 | 1.385 | −13.271 |
| ATOM | 2372 | C | TYR | 212 | −20.974 | 1.889 | −14.488 |
| ATOM | 2373 | O | TYR | 212 | −21.462 | 1.689 | −15.589 |
| ATOM | 2374 | CB | TYR | 212 | −22.413 | 2.582 | −12.579 |
| ATOM | 2375 | CG | TYR | 212 | −23.352 | 3.269 | −13.58 |
| ATOM | 2376 | CD1 | TYR | 212 | −24.657 | 2.797 | −13.758 |
| ATOM | 2377 | CD2 | TYR | 212 | −22.912 | 4.368 | −14.327 |
| ATOM | 2378 | CE1 | TYR | 212 | −25.515 | 3.415 | −14.673 |
| ATOM | 2379 | CE2 | TYR | 212 | −23.773 | 4.99 | −15.235 |
| ATOM | 2380 | CZ | TYR | 212 | −25.074 | 4.516 | −15.418 |
| ATOM | 2381 | OH | TYR | 212 | −25.908 | 5.151 | −16.344 |
| ATOM | 2382 | H | TYR | 212 | −20.742 | 0.953 | −11.421 |
| ATOM | 2383 | HB1 | TYR | 212 | −21.662 | 3.297 | −12.21 |
| ATOM | 2384 | HB2 | TYR | 212 | −22.992 | 2.227 | −11.711 |
| ATOM | 2385 | HD1 | TYR | 212 | −25.01 | 1.944 | −13.187 |
| ATOM | 2386 | HD2 | TYR | 212 | −21.899 | 4.741 | −14.208 |
| ATOM | 2387 | HE1 | TYR | 212 | −26.523 | 3.031 | −14.796 |
| ATOM | 2388 | HE2 | TYR | 212 | −23.431 | 5.847 | −15.806 |
| ATOM | 2389 | HH | TYR | 212 | −26.784 | 4.786 | −16.397 |
| ATOM | 2390 | HA | TYR | 212 | −22.482 | 0.662 | −13.59 |
| ATOM | 2391 | N | VAL | 213 | −19.793 | 2.526 | −14.326 |
| ATOM | 2392 | CA | VAL | 213 | −19.03 | 2.932 | −15.507 |
| ATOM | 2393 | C | VAL | 213 | −18.761 | 1.709 | −16.356 |
| ATOM | 2394 | O | VAL | 213 | −18.956 | 1.776 | −17.558 |
| ATOM | 2395 | CB | VAL | 213 | −17.714 | 3.688 | −15.15 |
| ATOM | 2396 | CG1 | VAL | 213 | −16.76 | 3.796 | −16.373 |
| ATOM | 2397 | CG2 | VAL | 213 | −18.037 | 5.11 | −14.611 |
| ATOM | 2398 | HB | VAL | 213 | −17.178 | 3.126 | −14.369 |
| ATOM | 2399 | HG23 | VAL | 213 | −18.493 | 5.726 | −15.402 |
| ATOM | 2400 | HG22 | VAL | 213 | −17.116 | 5.609 | −14.271 |
| ATOM | 2401 | HG21 | VAL | 213 | −18.736 | 5.066 | −13.764 |
| ATOM | 2402 | HG13 | VAL | 213 | −16.421 | 2.802 | −16.705 |
| ATOM | 2403 | HG12 | VAL | 213 | −15.867 | 4.386 | −16.113 |
| ATOM | 2404 | HG11 | VAL | 213 | −17.274 | 4.289 | −17.209 |
| ATOM | 2405 | H | VAL | 213 | −19.411 | 2.68 | −13.411 |
| ATOM | 2406 | HA | VAL | 213 | −19.657 | 3.613 | −16.104 |
| ATOM | 2407 | N | HIS | 214 | −18.326 | 0.566 | −15.786 |
| ATOM | 2408 | CA | HIS | 214 | −18.127 | −0.616 | −16.627 |
| ATOM | 2409 | C | HIS | 214 | −19.405 | −0.993 | −17.355 |
| ATOM | 2410 | O | HIS | 214 | −19.322 | −1.411 | −18.499 |
| ATOM | 2411 | CB | HIS | 214 | −17.627 | −1.85 | −15.828 |
| ATOM | 2412 | CG | HIS | 214 | −16.424 | −1.599 | −14.953 |
| ATOM | 2413 | ND1 | HIS | 214 | −15.592 | −0.584 | −15.05 |
| ATOM | 2414 | CD2 | HIS | 214 | −16.045 | −2.404 | −13.939 |
| ATOM | 2415 | CE1 | HIS | 214 | −14.678 | −0.68 | −14.136 |
| ATOM | 2416 | NE2 | HIS | 214 | −14.88 | −1.714 | −13.457 |
| ATOM | 2417 | HB2 | HIS | 214 | −18.432 | −2.214 | −15.175 |
| ATOM | 2418 | HB1 | HIS | 214 | −17.374 | −2.656 | −16.534 |
| ATOM | 2419 | HD1 | HIS | 214 | −15.65 | 0.18 | −15.737 |
| ATOM | 2420 | HD2 | HIS | 214 | −16.497 | −3.327 | −13.579 |
| ATOM | 2421 | HE1 | HIS | 214 | −13.858 | 0.019 | −13.971 |
| ATOM | 2422 | H | HIS | 214 | −18.167 | 0.51 | −14.796 |
| ATOM | 2423 | HA | HIS | 214 | −17.368 | −0.369 | −17.389 |
| ATOM | 2424 | N | MET | 215 | −20.592 | −0.86 | −16.717 |
| ATOM | 2425 | CA | MET | 215 | −21.857 | −1.208 | −17.376 |
| ATOM | 2426 | C | MET | 215 | −22.54 | −0.022 | −18.03 |
| ATOM | 2427 | O | MET | 215 | −23.634 | −0.222 | −18.535 |
| ATOM | 2428 | CB | MET | 215 | −22.826 | −1.9 | −16.365 |
| ATOM | 2429 | CG | MET | 215 | −22.52 | −3.409 | −16.164 |
| ATOM | 2430 | SD | MET | 215 | −20.764 | −3.676 | −15.759 |
| ATOM | 2431 | CE | MET | 215 | −20.17 | −4.633 | −17.189 |
| ATOM | 2432 | HB2 | MET | 215 | −22.787 | −1.374 | −15.398 |
| ATOM | 2433 | HB1 | MET | 215 | −23.869 | −1.848 | −16.718 |
| ATOM | 2434 | HG2 | MET | 215 | −22.778 | −3.977 | −17.072 |
| ATOM | 2435 | HG1 | MET | 215 | −23.129 | −3.797 | −15.332 |
| ATOM | 2436 | HE3 | MET | 215 | −20.659 | −5.619 | −17.203 |
| ATOM | 2437 | HE2 | MET | 215 | −19.083 | −4.768 | −17.102 |
| ATOM | 2438 | HE1 | MET | 215 | −20.388 | −4.098 | −18.124 |
| ATOM | 2439 | H | MET | 215 | −20.621 | −0.495 | −15.783 |
| ATOM | 2440 | HA | MET | 215 | −21.694 | −1.915 | −18.204 |
| ATOM | 2441 | LPD2 | MET | 215 | −20.697 | −4.029 | −15.182 |
| ATOM | 2442 | LPD1 | MET | 215 | −20.433 | −3.086 | −15.703 |
| ATOM | 2443 | N | PHE | 216 | −21.987 | 1.212 | −18.082 |
| ATOM | 2444 | CA | PHE | 216 | −22.74 | 2.33 | −18.668 |
| ATOM | 2445 | C | PHE | 216 | −22.832 | 2.279 | −20.182 |
| ATOM | 2446 | O | PHE | 216 | −22.507 | 3.263 | −20.829 |
| ATOM | 2447 | CB | PHE | 216 | −22.2 | 3.701 | −18.166 |
| ATOM | 2448 | CG | PHE | 216 | −20.81 | 4.144 | −18.662 |
| ATOM | 2449 | CD1 | PHE | 216 | −20.349 | 5.398 | −18.243 |
| ATOM | 2450 | CD2 | PHE | 216 | −19.992 | 3.376 | −19.503 |
| ATOM | 2451 | CE1 | PHE | 216 | −19.125 | 5.902 | −18.693 |
| ATOM | 2452 | CE2 | PHE | 216 | −18.771 | 3.878 | −19.961 |
| ATOM | 2453 | CZ | PHE | 216 | −18.34 | 5.147 | −19.567 |
| ATOM | 2454 | HB2 | PHE | 216 | −22.911 | 4.488 | −18.467 |
| ATOM | 2455 | HB1 | PHE | 216 | −22.168 | 3.678 | −17.067 |
| ATOM | 2456 | HD1 | PHE | 216 | −20.946 | 5.993 | −17.56 |
| ATOM | 2457 | HD2 | PHE | 216 | −20.28 | 2.379 | −19.814 |
| ATOM | 2458 | HE1 | PHE | 216 | −18.785 | 6.879 | −18.364 |
| ATOM | 2459 | HE2 | PHE | 216 | −18.154 | 3.28 | −20.624 |
| ATOM | 2460 | HZ | PHE | 216 | −17.398 | 5.543 | −19.935 |
| ATOM | 2461 | H | PHE | 216 | −21.083 | 1.387 | −17.687 |
| ATOM | 2462 | HA | PHE | 216 | −23.78 | 2.27 | −18.303 |
| ATOM | 2463 | N | LEU | 217 | −23.271 | 1.156 | −20.795 |
| ATOM | 2464 | CA | LEU | 217 | −23.214 | 1.044 | −22.253 |
| ATOM | 2465 | C | LEU | 217 | −24.105 | −0.079 | −22.729 |
| ATOM | 2466 | O | LEU | 217 | −25.013 | 0.131 | −23.517 |
| ATOM | 2467 | CB | LEU | 217 | −21.737 | 0.766 | −22.648 |
| ATOM | 2468 | CG | LEU | 217 | −21.51 | 0.448 | −24.158 |
| ATOM | 2469 | CD1 | LEU | 217 | −21.976 | 1.611 | −25.078 |
| ATOM | 2470 | CD2 | LEU | 217 | −20.005 | 0.147 | −24.408 |
| ATOM | 2471 | HB2 | LEU | 217 | −21.121 | 1.636 | −22.368 |
| ATOM | 2472 | HB1 | LEU | 217 | −21.39 | −0.093 | −22.051 |
| ATOM | 2473 | HG | LEU | 217 | −22.079 | −0.459 | −24.429 |
| ATOM | 2474 | HD13 | LEU | 217 | −23.066 | 1.747 | −25.027 |
| ATOM | 2475 | HD12 | LEU | 217 | −21.718 | 1.395 | −26.126 |
| ATOM | 2476 | HD11 | LEU | 217 | −21.486 | 2.552 | −24.784 |
| ATOM | 2477 | HD23 | LEU | 217 | −19.392 | 1.031 | −24.172 |
| ATOM | 2478 | HD22 | LEU | 217 | −19.834 | −0.124 | −25.462 |
| ATOM | 2479 | HD21 | LEU | 217 | −19.668 | −0.692 | −23.78 |
| ATOM | 2480 | H | LEU | 217 | −23.589 | 0.369 | −20.265 |
| ATOM | 2481 | HA | LEU | 217 | −23.567 | 1.984 | −22.708 |
| ATOM | 2482 | H9 | LEU | 217 | −23.943 | −1.096 | −22.374 |
| ATOM | 2483 | N | ALA | 239 | −36.516 | −5.492 | −20.37 |
| ATOM | 2484 | CA | ALA | 239 | −36.554 | −5.98 | −18.994 |
| ATOM | 2485 | C | ALA | 239 | −35.169 | −5.921 | −18.384 |
| ATOM | 2486 | O | ALA | 239 | −35.008 | −5.378 | −17.302 |
| ATOM | 2487 | CB | ALA | 239 | −37.089 | −7.434 | −18.919 |
| ATOM | 2488 | H | ALA | 239 | −37.091 | −5.919 | −21.07 |
| ATOM | 2489 | HB3 | ALA | 239 | −36.463 | −8.11 | −19.521 |
| ATOM | 2490 | HB2 | ALA | 239 | −38.121 | −7.479 | −19.303 |
| ATOM | 2491 | HB1 | ALA | 239 | −37.088 | −7.786 | −17.876 |
| ATOM | 2492 | HA | ALA | 239 | −37.237 | −5.335 | −18.418 |
| ATOM | 2493 | H10 | ALA | 239 | −35.977 | −4.677 | −20.59 |
| ATOM | 2494 | N | ASN | 240 | −34.147 | −6.478 | −19.072 |
| ATOM | 2495 | CA | ASN | 240 | −32.797 | −6.457 | −18.509 |
| ATOM | 2496 | C | ASN | 240 | −32.36 | −5.027 | −18.285 |
| ATOM | 2497 | O | ASN | 240 | −31.9 | −4.695 | −17.202 |
| ATOM | 2498 | CB | ASN | 240 | −31.781 | −7.168 | −19.446 |
| ATOM | 2499 | CG | ASN | 240 | −30.379 | −7.207 | −18.889 |
| ATOM | 2500 | OD1 | ASN | 240 | −30.168 | −6.839 | −17.745 |
| ATOM | 2501 | ND2 | ASN | 240 | −29.39 | −7.665 | −19.684 |
| ATOM | 2502 | HB2 | ASN | 240 | −32.106 | −8.208 | −19.603 |
| ATOM | 2503 | HB1 | ASN | 240 | −31.759 | −6.661 | −20.422 |
| ATOM | 2504 | HD21 | ASN | 240 | −29.565 | −7.969 | −20.62 |
| ATOM | 2505 | HD22 | ASN | 240 | −28.458 | −7.721 | −19.323 |
| ATOM | 2506 | H | ASN | 240 | −34.302 | −6.904 | −19.965 |
| ATOM | 2507 | HA | ASN | 240 | −32.825 | −6.992 | −17.547 |
| ATOM | 2508 | N | MET | 241 | −32.512 | −4.161 | −19.313 |
| ATOM | 2509 | CA | MET | 241 | −32.111 | −2.766 | −19.156 |
| ATOM | 2510 | C | MET | 241 | −33.046 | −2.107 | −18.169 |
| ATOM | 2511 | O | MET | 241 | −32.577 | −1.285 | −17.4 |
| ATOM | 2512 | CB | MET | 241 | −32.104 | −1.969 | −20.491 |
| ATOM | 2513 | CG | MET | 241 | −30.866 | −2.292 | −21.372 |
| ATOM | 2514 | SD | MET | 241 | −30.788 | −4.061 | −21.806 |
| ATOM | 2515 | CE | MET | 241 | −29.205 | −4.112 | −22.702 |
| ATOM | 2516 | HB2 | MET | 241 | −33.035 | −2.16 | −21.048 |
| ATOM | 2517 | HB1 | MET | 241 | −32.067 | −0.89 | −20.26 |
| ATOM | 2518 | HG2 | MET | 241 | −29.941 | −2.023 | −20.837 |
| ATOM | 2519 | HG1 | MET | 241 | −30.909 | −1.71 | −22.306 |
| ATOM | 2520 | HE3 | MET | 241 | −29.245 | −3.437 | −23.57 |
| ATOM | 2521 | HE2 | MET | 241 | −29.022 | −5.139 | −23.048 |
| ATOM | 2522 | HE1 | MET | 241 | −28.387 | −3.804 | −22.034 |
| ATOM | 2523 | H | MET | 241 | −32.947 | −4.449 | −20.167 |
| ATOM | 2524 | HA | MET | 241 | −31.094 | −2.73 | −18.731 |
| ATOM | 2525 | LPD2 | MET | 241 | −31.307 | −4.245 | −22.203 |
| ATOM | 2526 | LPD1 | MET | 241 | −30.776 | −4.455 | −21.253 |
| ATOM | 2527 | N | LYS | 242 | −34.357 | −2.439 | −18.135 |
| ATOM | 2528 | CA | LYS | 242 | −35.204 | −1.854 | −17.096 |
| ATOM | 2529 | C | LYS | 242 | −34.586 | −2.173 | −15.753 |
| ATOM | 2530 | O | LYS | 242 | −34.551 | −1.302 | −14.898 |
| ATOM | 2531 | CB | LYS | 242 | −36.681 | −2.333 | −17.165 |
| ATOM | 2532 | CG | LYS | 242 | −37.629 | −1.546 | −16.203 |
| ATOM | 2533 | CD | LYS | 242 | −38.269 | −2.414 | −15.08 |
| ATOM | 2534 | CE | LYS | 242 | −37.232 | −2.959 | −14.06 |
| ATOM | 2535 | NZ | LYS | 242 | −37.883 | −3.605 | −12.895 |
| ATOM | 2536 | HB2 | LYS | 242 | −37.016 | −2.185 | −18.206 |
| ATOM | 2537 | HB1 | LYS | 242 | −36.722 | −3.412 | −16.965 |
| ATOM | 2538 | HG2 | LYS | 242 | −38.461 | −1.122 | −16.792 |
| ATOM | 2539 | HG1 | LYS | 242 | −37.107 | −0.694 | −15.738 |
| ATOM | 2540 | HD2 | LYS | 242 | −38.824 | −3.254 | −15.531 |
| ATOM | 2541 | HD1 | LYS | 242 | −38.995 | −1.786 | −14.538 |
| ATOM | 2542 | HE2 | LYS | 242 | −36.614 | −2.123 | −13.701 |
| ATOM | 2543 | HE1 | LYS | 242 | −36.571 | −3.689 | −14.556 |
| ATOM | 2544 | HZ3 | LYS | 242 | −38.446 | −2.905 | −12.295 |
| ATOM | 2545 | HZ2 | LYS | 242 | −37.151 | −4.035 | −12.227 |
| ATOM | 2546 | HZ1 | LYS | 242 | −38.556 | −4.399 | −13.188 |
| ATOM | 2547 | H | LYS | 242 | −34.749 | −3.099 | −18.776 |
| ATOM | 2548 | HA | LYS | 242 | −35.196 | −0.764 | −17.254 |
| ATOM | 2549 | N | GLY | 243 | −34.07 | −3.409 | −15.556 |
| ATOM | 2550 | CA | GLY | 243 | −33.344 | −3.717 | −14.325 |
| ATOM | 2551 | C | GLY | 243 | −32.26 | −2.695 | −14.07 |
| ATOM | 2552 | O | GLY | 243 | −32.153 | −2.186 | −12.965 |
| ATOM | 2553 | H | GLY | 243 | −34.147 | −4.125 | −16.258 |
| ATOM | 2554 | HA1 | GLY | 243 | −34.048 | −3.76 | −13.481 |
| ATOM | 2555 | HA2 | GLY | 243 | −32.848 | −4.698 | −14.398 |
| ATOM | 2556 | N | ALA | 244 | −31.433 | −2.383 | −15.092 |
| ATOM | 2557 | CA | ALA | 244 | −30.395 | −1.371 | −14.899 |
| ATOM | 2558 | C | ALA | 244 | −31.005 | −0.012 | −14.645 |
| ATOM | 2559 | O | ALA | 244 | −30.48 | 0.726 | −13.827 |
| ATOM | 2560 | CB | ALA | 244 | −29.468 | −1.248 | −16.138 |
| ATOM | 2561 | HB3 | ALA | 244 | −30.036 | −0.899 | −17.014 |
| ATOM | 2562 | HB2 | ALA | 244 | −29.014 | −2.219 | −16.382 |
| ATOM | 2563 | HB1 | ALA | 244 | −28.662 | −0.525 | −15.937 |
| ATOM | 2564 | H | ALA | 244 | −31.539 | −2.814 | −15.991 |
| ATOM | 2565 | HA | ALA | 244 | −29.781 | −1.655 | −14.027 |
| ATOM | 2566 | N | ILE | 245 | −32.103 | 0.365 | −15.334 |
| ATOM | 2567 | CA | ILE | 245 | −32.641 | 1.711 | −15.151 |
| ATOM | 2568 | C | ILE | 245 | −33.213 | 1.826 | −13.758 |
| ATOM | 2569 | O | ILE | 245 | −32.877 | 2.775 | −13.069 |
| ATOM | 2570 | CB | ILE | 245 | −33.691 | 2.103 | −16.234 |
| ATOM | 2571 | CG1 | ILE | 245 | −33.012 | 2.169 | −17.639 |
| ATOM | 2572 | CG2 | ILE | 245 | −34.36 | 3.458 | −15.863 |
| ATOM | 2573 | CD1 | ILE | 245 | −34.018 | 2.183 | −18.823 |
| ATOM | 2574 | HB | ILE | 245 | −34.474 | 1.329 | −16.241 |
| ATOM | 2575 | HG23 | ILE | 245 | −34.951 | 3.366 | −14.94 |
| ATOM | 2576 | HG22 | ILE | 245 | −35.041 | 3.789 | −16.654 |
| ATOM | 2577 | HG21 | ILE | 245 | −33.597 | 4.238 | −15.716 |
| ATOM | 2578 | HG12 | ILE | 245 | −32.365 | 1.29 | −17.785 |
| ATOM | 2579 | HG11 | ILE | 245 | −32.369 | 3.062 | −17.696 |
| ATOM | 2580 | HD13 | ILE | 245 | −34.691 | 1.314 | −18.767 |
| ATOM | 2581 | HD12 | ILE | 245 | −33.468 | 2.131 | −19.777 |
| ATOM | 2582 | HD11 | ILE | 245 | −34.624 | 3.1 | −18.839 |
| ATOM | 2583 | H | ILE | 245 | −32.555 | −0.269 | −15.964 |
| ATOM | 2584 | HA | ILE | 245 | −31.81 | 2.43 | −15.24 |
| ATOM | 2585 | N | THR | 246 | −34.077 | 0.894 | −13.297 |
| ATOM | 2586 | CA | THR | 246 | −34.577 | 1.028 | −11.929 |
| ATOM | 2587 | C | THR | 246 | −33.398 | 1.055 | −10.984 |
| ATOM | 2588 | O | THR | 246 | −33.463 | 1.792 | −10.015 |
| ATOM | 2589 | CB | THR | 246 | −35.641 | −0.017 | −11.495 |
| ATOM | 2590 | OG1 | THR | 246 | −36.251 | 0.439 | −10.273 |
| ATOM | 2591 | CG2 | THR | 246 | −35.035 | −1.425 | −11.276 |
| ATOM | 2592 | HB | THR | 246 | −36.413 | −0.054 | −12.284 |
| ATOM | 2593 | HG22 | THR | 246 | −35.832 | −2.141 | −11.026 |
| ATOM | 2594 | HG21 | THR | 246 | −34.309 | −1.413 | −10.45 |
| ATOM | 2595 | HG23 | THR | 246 | −34.532 | −1.765 | −12.187 |
| ATOM | 2596 | HG1 | THR | 246 | −36.952 | −0.128 | −9.964 |
| ATOM | 2597 | H | THR | 246 | −34.352 | 0.112 | −13.859 |
| ATOM | 2598 | HA | THR | 246 | −35.081 | 2.007 | −11.866 |
| ATOM | 2599 | N | LEU | 247 | −32.299 | 0.304 | −11.237 |
| ATOM | 2600 | CA | LEU | 247 | −31.116 | 0.465 | −10.388 |
| ATOM | 2601 | C | LEU | 247 | −30.736 | 1.931 | −10.467 |
| ATOM | 2602 | O | LEU | 247 | −30.704 | 2.591 | −9.441 |
| ATOM | 2603 | CB | LEU | 247 | −29.944 | −0.481 | −10.798 |
| ATOM | 2604 | CG | LEU | 247 | −28.825 | −0.675 | −9.728 |
| ATOM | 2605 | CD1 | LEU | 247 | −27.729 | −1.636 | −10.268 |
| ATOM | 2606 | CD2 | LEU | 247 | −28.145 | 0.655 | −9.306 |
| ATOM | 2607 | HB2 | LEU | 247 | −30.376 | −1.476 | −10.994 |
| ATOM | 2608 | HB1 | LEU | 247 | −29.479 | −0.132 | −11.729 |
| ATOM | 2609 | HG | LEU | 247 | −29.275 | −1.135 | −8.832 |
| ATOM | 2610 | HD13 | LEU | 247 | −28.163 | −2.581 | −10.619 |
| ATOM | 2611 | HD12 | LEU | 247 | −27.01 | −1.864 | −9.467 |
| ATOM | 2612 | HD11 | LEU | 247 | −27.187 | −1.174 | −11.107 |
| ATOM | 2613 | HD23 | LEU | 247 | −27.811 | 1.221 | −10.189 |
| ATOM | 2614 | HD22 | LEU | 247 | −27.276 | 0.465 | −8.657 |
| ATOM | 2615 | HD21 | LEU | 247 | −28.847 | 1.266 | −8.734 |
| ATOM | 2616 | H | LEU | 247 | −32.255 | −0.317 | −12.024 |
| ATOM | 2617 | HA | LEU | 247 | −31.401 | 0.211 | −9.354 |
| ATOM | 2618 | N | THR | 248 | −30.469 | 2.47 | −11.68 |
| ATOM | 2619 | CA | THR | 248 | −30.054 | 3.868 | −11.798 |
| ATOM | 2620 | C | THR | 248 | −30.951 | 4.789 | −10.999 |
| ATOM | 2621 | O | THR | 248 | −30.441 | 5.675 | −10.331 |
| ATOM | 2622 | CB | THR | 248 | −30.032 | 4.332 | −13.281 |
| ATOM | 2623 | OG1 | THR | 248 | −29.277 | 3.361 | −14.029 |
| ATOM | 2624 | CG2 | THR | 248 | −29.389 | 5.737 | −13.437 |
| ATOM | 2625 | HB | THR | 248 | −31.067 | 4.37 | −13.661 |
| ATOM | 2626 | HG22 | THR | 248 | −29.384 | 6.034 | −14.497 |
| ATOM | 2627 | HG21 | THR | 248 | −28.35 | 5.721 | −13.074 |
| ATOM | 2628 | HG23 | THR | 248 | −29.954 | 6.493 | −12.87 |
| ATOM | 2629 | HG1 | THR | 248 | −29.252 | 3.543 | −14.962 |
| ATOM | 2630 | H | THR | 248 | −30.527 | 1.924 | −12.519 |
| ATOM | 2631 | HA | THR | 248 | −29.02 | 3.931 | −11.426 |
| ATOM | 2632 | N | ILE | 249 | −32.29 | 4.608 | −11.048 |
| ATOM | 2633 | CA | ILE | 249 | −33.168 | 5.504 | −10.292 |
| ATOM | 2634 | C | ILE | 249 | −33.013 | 5.219 | −8.813 |
| ATOM | 2635 | O | ILE | 249 | −32.851 | 6.149 | −8.039 |
| ATOM | 2636 | CB | ILE | 249 | −34.677 | 5.393 | −10.673 |
| ATOM | 2637 | CG1 | ILE | 249 | −34.948 | 5.438 | −12.21 |
| ATOM | 2638 | CG2 | ILE | 249 | −35.506 | 6.489 | −9.94 |
| ATOM | 2639 | CD1 | ILE | 249 | −34.201 | 6.563 | −12.973 |
| ATOM | 2640 | H | ILE | 249 | −32.681 | 3.844 | −11.565 |
| ATOM | 2641 | HB | ILE | 249 | −35.046 | 4.415 | −10.315 |
| ATOM | 2642 | HG12 | ILE | 249 | −36.031 | 5.561 | −12.366 |
| ATOM | 2643 | HG11 | ILE | 249 | −34.675 | 4.48 | −12.673 |
| ATOM | 2644 | HG23 | ILE | 249 | −35.356 | 6.436 | −8.854 |
| ATOM | 2645 | HG22 | ILE | 249 | −36.582 | 6.36 | −10.133 |
| ATOM | 2646 | HG21 | ILE | 249 | −35.211 | 7.496 | −10.268 |
| ATOM | 2647 | HD13 | ILE | 249 | −34.437 | 7.553 | −12.558 |
| ATOM | 2648 | HD12 | ILE | 249 | −34.503 | 6.554 | −14.033 |
| ATOM | 2649 | HD11 | ILE | 249 | −33.112 | 6.409 | −12.934 |
| ATOM | 2650 | HA | ILE | 249 | −32.851 | 6.544 | −10.475 |
| ATOM | 2651 | N | LEU | 250 | −33.07 | 3.934 | −8.399 |
| ATOM | 2652 | CA | LEU | 250 | −33 | 3.613 | −6.976 |
| ATOM | 2653 | C | LEU | 250 | −31.715 | 4.175 | −6.4 |
| ATOM | 2654 | O | LEU | 250 | −31.768 | 4.891 | −5.412 |
| ATOM | 2655 | CB | LEU | 250 | −33.067 | 2.083 | −6.709 |
| ATOM | 2656 | CG | LEU | 250 | −34.445 | 1.416 | −7.019 |
| ATOM | 2657 | CD1 | LEU | 250 | −34.275 | −0.117 | −7.226 |
| ATOM | 2658 | CD2 | LEU | 250 | −35.478 | 1.666 | −5.885 |
| ATOM | 2659 | HB2 | LEU | 250 | −32.285 | 1.616 | −7.326 |
| ATOM | 2660 | HB1 | LEU | 250 | −32.825 | 1.903 | −5.65 |
| ATOM | 2661 | HG | LEU | 250 | −34.86 | 1.83 | −7.951 |
| ATOM | 2662 | HD13 | LEU | 250 | −33.596 | −0.325 | −8.067 |
| ATOM | 2663 | HD12 | LEU | 250 | −35.244 | −0.589 | −7.45 |
| ATOM | 2664 | HD11 | LEU | 250 | −33.859 | −0.581 | −6.318 |
| ATOM | 2665 | HD23 | LEU | 250 | −35.111 | 1.242 | −4.94 |
| ATOM | 2666 | HD22 | LEU | 250 | −36.437 | 1.183 | −6.128 |
| ATOM | 2667 | HD21 | LEU | 250 | −35.661 | 2.739 | −5.735 |
| ATOM | 2668 | H | LEU | 250 | −33.155 | 3.189 | −9.061 |
| ATOM | 2669 | HA | LEU | 250 | −33.851 | 4.089 | −6.469 |
| ATOM | 2670 | N | ILE | 251 | −30.538 | 3.876 | −6.997 |
| ATOM | 2671 | CA | ILE | 251 | −29.301 | 4.454 | −6.467 |
| ATOM | 2672 | C | ILE | 251 | −29.315 | 5.953 | −6.671 |
| ATOM | 2673 | O | ILE | 251 | −28.808 | 6.674 | −5.825 |
| ATOM | 2674 | CB | ILE | 251 | −27.997 | 3.794 | −7.009 |
| ATOM | 2675 | CG1 | ILE | 251 | −26.769 | 4.104 | −6.096 |
| ATOM | 2676 | CG2 | ILE | 251 | −27.722 | 4.202 | −8.482 |
| ATOM | 2677 | CD1 | ILE | 251 | −25.496 | 3.322 | −6.521 |
| ATOM | 2678 | HB | ILE | 251 | −28.159 | 2.705 | −6.963 |
| ATOM | 2679 | HG23 | ILE | 251 | −28.626 | 4.069 | −9.085 |
| ATOM | 2680 | HG22 | ILE | 251 | −26.936 | 3.579 | −8.932 |
| ATOM | 2681 | HG21 | ILE | 251 | −27.417 | 5.257 | −8.544 |
| ATOM | 2682 | HG12 | ILE | 251 | −26.999 | 3.813 | −5.058 |
| ATOM | 2683 | HG11 | ILE | 251 | −26.554 | 5.185 | −6.101 |
| ATOM | 2684 | HD13 | ILE | 251 | −25.731 | 2.255 | −6.651 |
| ATOM | 2685 | HD12 | ILE | 251 | −24.718 | 3.409 | −5.749 |
| ATOM | 2686 | HD11 | ILE | 251 | −25.078 | 3.711 | −7.461 |
| ATOM | 2687 | H | ILE | 251 | −30.509 | 3.297 | −7.815 |
| ATOM | 2688 | HA | ILE | 251 | −29.328 | 4.261 | −5.389 |
| ATOM | 2689 | N | GLY | 252 | −29.903 | 6.453 | −7.781 |
| ATOM | 2690 | CA | GLY | 252 | −29.984 | 7.896 | −7.975 |
| ATOM | 2691 | C | GLY | 252 | −30.655 | 8.56 | −6.798 |
| ATOM | 2692 | O | GLY | 252 | −30.162 | 9.58 | −6.343 |
| ATOM | 2693 | H | GLY | 252 | −30.302 | 5.862 | −8.487 |
| ATOM | 2694 | HA1 | GLY | 252 | −28.967 | 8.298 | −8.106 |
| ATOM | 2695 | HA2 | GLY | 252 | −30.57 | 8.146 | −8.874 |
| ATOM | 2696 | N | VAL | 253 | −31.785 | 8.018 | −6.287 |
| ATOM | 2697 | CA | VAL | 253 | −32.399 | 8.624 | −5.105 |
| ATOM | 2698 | C | VAL | 253 | −31.65 | 8.229 | −3.85 |
| ATOM | 2699 | O | VAL | 253 | −31.634 | 9.019 | −2.919 |
| ATOM | 2700 | CB | VAL | 253 | −33.923 | 8.363 | −4.924 |
| ATOM | 2701 | CG1 | VAL | 253 | −34.715 | 8.833 | −6.176 |
| ATOM | 2702 | CG2 | VAL | 253 | −34.251 | 6.884 | −4.59 |
| ATOM | 2703 | HB | VAL | 253 | −34.262 | 8.968 | −4.063 |
| ATOM | 2704 | HG23 | VAL | 253 | −34.015 | 6.236 | −5.441 |
| ATOM | 2705 | HG22 | VAL | 253 | −35.323 | 6.788 | −4.374 |
| ATOM | 2706 | HG21 | VAL | 253 | −33.696 | 6.532 | −3.707 |
| ATOM | 2707 | HG13 | VAL | 253 | −34.494 | 9.888 | −6.4 |
| ATOM | 2708 | HG12 | VAL | 253 | −35.798 | 8.743 | −6 |
| ATOM | 2709 | HG11 | VAL | 253 | −34.453 | 8.227 | −7.056 |
| ATOM | 2710 | H | VAL | 253 | −32.176 | 7.182 | −6.681 |
| ATOM | 2711 | HA | VAL | 253 | −32.306 | 9.716 | −5.212 |
| ATOM | 2712 | N | PHE | 254 | −31.028 | 7.028 | −3.779 |
| ATOM | 2713 | CA | PHE | 254 | −30.301 | 6.66 | −2.564 |
| ATOM | 2714 | C | PHE | 254 | −29.414 | 7.8 | −2.118 |
| ATOM | 2715 | O | PHE | 254 | −29.435 | 8.16 | −0.952 |
| ATOM | 2716 | CB | PHE | 254 | −29.411 | 5.4 | −2.748 |
| ATOM | 2717 | CG | PHE | 254 | −28.611 | 5.105 | −1.471 |
| ATOM | 2718 | CD1 | PHE | 254 | −29.059 | 4.163 | −0.538 |
| ATOM | 2719 | CD2 | PHE | 254 | −27.415 | 5.792 | −1.232 |
| ATOM | 2720 | CE1 | PHE | 254 | −28.389 | 4.007 | 0.68 |
| ATOM | 2721 | CE2 | PHE | 254 | −26.753 | 5.652 | −0.011 |
| ATOM | 2722 | CZ | PHE | 254 | −27.265 | 4.789 | 0.961 |
| ATOM | 2723 | H | PHE | 254 | −31.08 | 6.365 | −4.528 |
| ATOM | 2724 | HB2 | PHE | 254 | −28.689 | 5.574 | −3.557 |
| ATOM | 2725 | HB1 | PHE | 254 | −30.037 | 4.535 | −3.019 |
| ATOM | 2726 | HD1 | PHE | 254 | −29.934 | 3.556 | −0.748 |
| ATOM | 2727 | HD2 | PHE | 254 | −26.99 | 6.437 | −1.993 |
| ATOM | 2728 | HE1 | PHE | 254 | −28.747 | 3.287 | 1.409 |
| ATOM | 2729 | HE2 | PHE | 254 | −25.844 | 6.211 | 0.18 |
| ATOM | 2730 | HZ | PHE | 254 | −26.786 | 4.735 | 1.929 |
| ATOM | 2731 | HA | PHE | 254 | −31.044 | 6.436 | −1.782 |
| ATOM | 2732 | N | VAL | 255 | −28.606 | 8.39 | −3.028 |
| ATOM | 2733 | CA | VAL | 255 | −27.677 | 9.422 | −2.572 |
| ATOM | 2734 | C | VAL | 255 | −28.431 | 10.579 | −1.954 |
| ATOM | 2735 | O | VAL | 255 | −28.038 | 10.998 | −0.877 |
| ATOM | 2736 | CB | VAL | 255 | −26.617 | 9.897 | −3.614 |
| ATOM | 2737 | CG1 | VAL | 255 | −25.686 | 8.723 | −4.032 |
| ATOM | 2738 | CG2 | VAL | 255 | −27.234 | 10.573 | −4.869 |
| ATOM | 2739 | HB | VAL | 255 | −25.986 | 10.656 | −3.118 |
| ATOM | 2740 | HG23 | VAL | 255 | −27.849 | 9.86 | −5.433 |
| ATOM | 2741 | HG22 | VAL | 255 | −26.435 | 10.932 | −5.535 |
| ATOM | 2742 | HG21 | VAL | 255 | −27.851 | 11.439 | −4.59 |
| ATOM | 2743 | HG13 | VAL | 255 | −25.184 | 8.302 | −3.148 |
| ATOM | 2744 | HG12 | VAL | 255 | −24.908 | 9.077 | −4.726 |
| ATOM | 2745 | HG11 | VAL | 255 | −26.253 | 7.922 | −4.529 |
| ATOM | 2746 | H | VAL | 255 | −28.627 | 8.115 | −3.991 |
| ATOM | 2747 | HA | VAL | 255 | −27.093 | 8.972 | −1.75 |
| ATOM | 2748 | N | VAL | 256 | −29.513 | 11.126 | −2.553 |
| ATOM | 2749 | CA | VAL | 256 | −30.177 | 12.257 | −1.898 |
| ATOM | 2750 | C | VAL | 256 | −30.844 | 11.779 | −0.628 |
| ATOM | 2751 | O | VAL | 256 | −30.892 | 12.531 | 0.331 |
| ATOM | 2752 | CB | VAL | 256 | −31.186 | 13.066 | −2.77 |
| ATOM | 2753 | CG1 | VAL | 256 | −30.545 | 13.501 | −4.117 |
| ATOM | 2754 | CG2 | VAL | 256 | −32.525 | 12.318 | −3.011 |
| ATOM | 2755 | HB | VAL | 256 | −31.442 | 13.984 | −2.21 |
| ATOM | 2756 | HG23 | VAL | 256 | −32.341 | 11.375 | −3.536 |
| ATOM | 2757 | HG22 | VAL | 256 | −33.2 | 12.931 | −3.629 |
| ATOM | 2758 | HG21 | VAL | 256 | −33.041 | 12.098 | −2.064 |
| ATOM | 2759 | HG13 | VAL | 256 | −29.59 | 14.019 | −3.938 |
| ATOM | 2760 | HG12 | VAL | 256 | −31.216 | 14.192 | −4.652 |
| ATOM | 2761 | HG11 | VAL | 256 | −30.358 | 12.636 | −4.77 |
| ATOM | 2762 | H | VAL | 256 | −29.893 | 10.732 | −3.392 |
| ATOM | 2763 | HA | VAL | 256 | −29.39 | 12.974 | −1.606 |
| ATOM | 2764 | N | CYS | 257 | −31.374 | 10.536 | −0.595 |
| ATOM | 2765 | CA | CYS | 257 | −32.021 | 10.046 | 0.62 |
| ATOM | 2766 | C | CYS | 257 | −31.038 | 9.952 | 1.77 |
| ATOM | 2767 | O | CYS | 257 | −31.35 | 10.418 | 2.855 |
| ATOM | 2768 | CB | CYS | 257 | −32.668 | 8.658 | 0.358 |
| ATOM | 2769 | SG | CYS | 257 | −33.268 | 7.875 | 1.887 |
| ATOM | 2770 | H | CYS | 257 | −31.332 | 9.936 | −1.398 |
| ATOM | 2771 | HB2 | CYS | 257 | −31.926 | 7.969 | −0.065 |
| ATOM | 2772 | HB1 | CYS | 257 | −33.499 | 8.751 | −0.36 |
| ATOM | 2773 | HA | CYS | 257 | −32.826 | 10.745 | 0.894 |
| ATOM | 2774 | HG | CYS | 257 | −33.883 | 8.611 | 2.194 |
| ATOM | 2775 | LPG2 | CYS | 257 | −32.767 | 7.758 | 2.33 |
| ATOM | 2776 | LPG1 | CYS | 257 | −33.602 | 7.296 | 1.765 |
| ATOM | 2777 | N | TRP | 258 | −29.854 | 9.334 | 1.557 |
| ATOM | 2778 | CA | TRP | 258 | −28.916 | 9.088 | 2.66 |
| ATOM | 2779 | C | TRP | 258 | −27.758 | 10.065 | 2.717 |
| ATOM | 2780 | O | TRP | 258 | −27.364 | 10.446 | 3.809 |
| ATOM | 2781 | CB | TRP | 258 | −28.33 | 7.665 | 2.493 |
| ATOM | 2782 | CG | TRP | 258 | −29.417 | 6.629 | 2.607 |
| ATOM | 2783 | CD1 | TRP | 258 | −30.183 | 6.118 | 1.63 |
| ATOM | 2784 | CD2 | TRP | 258 | −29.844 | 5.949 | 3.881 |
| ATOM | 2785 | NE1 | TRP | 258 | −30.996 | 5.22 | 2.124 |
| ATOM | 2786 | CE2 | TRP | 258 | −30.82 | 5.072 | 3.45 |
| ATOM | 2787 | CE3 | TRP | 258 | −29.461 | 6.043 | 5.216 |
| ATOM | 2788 | CZ2 | TRP | 258 | −31.459 | 4.198 | 4.328 |
| ATOM | 2789 | CZ3 | TRP | 258 | −30.08 | 5.158 | 6.107 |
| ATOM | 2790 | CH2 | TRP | 258 | −31.06 | 4.25 | 5.67 |
| ATOM | 2791 | H | TRP | 258 | −29.623 | 9.009 | 0.636 |
| ATOM | 2792 | HB2 | TRP | 258 | −27.572 | 7.462 | 3.266 |
| ATOM | 2793 | HB1 | TRP | 258 | −27.84 | 7.59 | 1.511 |
| ATOM | 2794 | HD1 | TRP | 258 | −30.129 | 6.402 | 0.585 |
| ATOM | 2795 | HE1 | TRP | 258 | −31.678 | 4.684 | 1.566 |
| ATOM | 2796 | HE3 | TRP | 258 | −28.718 | 6.763 | 5.539 |
| ATOM | 2797 | HZ2 | TRP | 258 | −32.225 | 3.51 | 3.986 |
| ATOM | 2798 | HZ3 | TRP | 258 | −29.789 | 5.176 | 7.151 |
| ATOM | 2799 | HH2 | TRP | 258 | −31.518 | 3.571 | 6.38 |
| ATOM | 2800 | HA | TRP | 258 | −29.431 | 9.116 | 3.635 |
| ATOM | 2801 | N | ALA | 259 | −27.177 | 10.475 | 1.566 |
| ATOM | 2802 | CA | ALA | 259 | −25.984 | 11.316 | 1.608 |
| ATOM | 2803 | C | ALA | 259 | −26.092 | 12.521 | 2.519 |
| ATOM | 2804 | O | ALA | 259 | −25.139 | 12.698 | 3.259 |
| ATOM | 2805 | CB | ALA | 259 | −25.488 | 11.797 | 0.218 |
| ATOM | 2806 | HB3 | ALA | 259 | −26.191 | 12.512 | −0.229 |
| ATOM | 2807 | HB2 | ALA | 259 | −25.366 | 10.94 | −0.463 |
| ATOM | 2808 | HB1 | ALA | 259 | −24.512 | 12.299 | 0.32 |
| ATOM | 2809 | H | ALA | 259 | −27.516 | 10.183 | 0.67 |
| ATOM | 2810 | HA | ALA | 259 | −25.186 | 10.663 | 2.004 |
| ATOM | 2811 | N | PRO | 260 | −27.131 | 13.394 | 2.56 |
| ATOM | 2812 | CA | PRO | 260 | −27.038 | 14.558 | 3.433 |
| ATOM | 2813 | C | PRO | 260 | −26.922 | 14.168 | 4.891 |
| ATOM | 2814 | O | PRO | 260 | −26.236 | 14.864 | 5.622 |
| ATOM | 2815 | CB | PRO | 260 | −28.348 | 15.302 | 3.074 |
| ATOM | 2816 | CG | PRO | 260 | −29.284 | 14.2 | 2.527 |
| ATOM | 2817 | CD | PRO | 260 | −28.334 | 13.258 | 1.748 |
| ATOM | 2818 | HB2 | PRO | 260 | −28.152 | 16.014 | 2.255 |
| ATOM | 2819 | HB1 | PRO | 260 | −28.779 | 15.855 | 3.922 |
| ATOM | 2820 | HG1 | PRO | 260 | −29.738 | 13.659 | 3.374 |
| ATOM | 2821 | HG2 | PRO | 260 | −30.093 | 14.605 | 1.897 |
| ATOM | 2822 | HD1 | PRO | 260 | −28.769 | 12.253 | 1.701 |
| ATOM | 2823 | HD2 | PRO | 260 | −28.154 | 13.641 | 0.732 |
| ATOM | 2824 | HA | PRO | 260 | −26.166 | 15.174 | 3.156 |
| ATOM | 2825 | N | PHE | 261 | −27.551 | 13.064 | 5.349 |
| ATOM | 2826 | CA | PHE | 261 | −27.38 | 12.651 | 6.745 |
| ATOM | 2827 | C | PHE | 261 | −25.974 | 12.133 | 6.948 |
| ATOM | 2828 | O | PHE | 261 | −25.336 | 12.497 | 7.924 |
| ATOM | 2829 | CB | PHE | 261 | −28.405 | 11.545 | 7.1 |
| ATOM | 2830 | CG | PHE | 261 | −28.353 | 11.116 | 8.575 |
| ATOM | 2831 | CD1 | PHE | 261 | −29.014 | 11.859 | 9.56 |
| ATOM | 2832 | CD2 | PHE | 261 | −27.673 | 9.954 | 8.953 |
| ATOM | 2833 | CE1 | PHE | 261 | −29.118 | 11.364 | 10.863 |
| ATOM | 2834 | CE2 | PHE | 261 | −27.888 | 9.392 | 10.214 |
| ATOM | 2835 | CZ | PHE | 261 | −28.604 | 10.104 | 11.18 |
| ATOM | 2836 | HB2 | PHE | 261 | −29.427 | 11.897 | 6.89 |
| ATOM | 2837 | HB1 | PHE | 261 | −28.218 | 10.682 | 6.445 |
| ATOM | 2838 | HD1 | PHE | 261 | −29.455 | 12.819 | 9.322 |
| ATOM | 2839 | HD2 | PHE | 261 | −26.987 | 9.477 | 8.265 |
| ATOM | 2840 | HE1 | PHE | 261 | −29.602 | 11.957 | 11.63 |
| ATOM | 2841 | HE2 | PHE | 261 | −27.503 | 8.403 | 10.443 |
| ATOM | 2842 | H15 | PHE | 261 | −28.757 | 9.681 | 12.168 |
| ATOM | 2843 | H | PHE | 261 | −28.078 | 12.481 | 4.728 |
| ATOM | 2844 | HA | PHE | 261 | −27.555 | 13.507 | 7.417 |
| ATOM | 2845 | N | PHE | 262 | −25.461 | 11.283 | 6.03 |
| ATOM | 2846 | CA | PHE | 262 | −24.092 | 10.784 | 6.184 |
| ATOM | 2847 | C | PHE | 262 | −23.102 | 11.918 | 5.982 |
| ATOM | 2848 | O | PHE | 262 | −22.035 | 11.881 | 6.576 |
| ATOM | 2849 | CB | PHE | 262 | −23.791 | 9.561 | 5.267 |
| ATOM | 2850 | CG | PHE | 262 | −24.294 | 8.266 | 5.934 |
| ATOM | 2851 | CD1 | PHE | 262 | −25.646 | 8.106 | 6.259 |
| ATOM | 2852 | CD2 | PHE | 262 | −23.406 | 7.231 | 6.253 |
| ATOM | 2853 | CE1 | PHE | 262 | −26.05 | 7.075 | 7.112 |
| ATOM | 2854 | CE2 | PHE | 262 | −23.813 | 6.167 | 7.063 |
| ATOM | 2855 | CZ | PHE | 262 | −25.127 | 6.115 | 7.535 |
| ATOM | 2856 | HB2 | PHE | 262 | −24.245 | 9.678 | 4.271 |
| ATOM | 2857 | HB1 | PHE | 262 | −22.7 | 9.494 | 5.126 |
| ATOM | 2858 | HD1 | PHE | 262 | −26.392 | 8.782 | 5.859 |
| ATOM | 2859 | HD2 | PHE | 262 | −22.392 | 7.243 | 5.877 |
| ATOM | 2860 | HE1 | PHE | 262 | −27.079 | 7.018 | 7.45 |
| ATOM | 2861 | HE2 | PHE | 262 | −23.108 | 5.385 | 7.329 |
| ATOM | 2862 | HZ | PHE | 262 | −25.431 | 5.331 | 8.221 |
| ATOM | 2863 | H | PHE | 262 | −25.983 | 11.04 | 5.207 |
| ATOM | 2864 | HA | PHE | 262 | −23.963 | 10.441 | 7.223 |
| ATOM | 2865 | N | LEU | 263 | −23.424 | 12.951 | 5.172 |
| ATOM | 2866 | CA | LEU | 263 | −22.537 | 14.107 | 5.053 |
| ATOM | 2867 | C | LEU | 263 | −22.597 | 14.814 | 6.389 |
| ATOM | 2868 | O | LEU | 263 | −21.57 | 15.05 | 7.004 |
| ATOM | 2869 | CB | LEU | 263 | −22.967 | 15.041 | 3.882 |
| ATOM | 2870 | CG | LEU | 263 | −21.911 | 16.095 | 3.428 |
| ATOM | 2871 | CD1 | LEU | 263 | −22.489 | 16.92 | 2.242 |
| ATOM | 2872 | CD2 | LEU | 263 | −21.473 | 17.057 | 4.566 |
| ATOM | 2873 | HB2 | LEU | 263 | −23.173 | 14.399 | 3.011 |
| ATOM | 2874 | HB1 | LEU | 263 | −23.902 | 15.556 | 4.148 |
| ATOM | 2875 | HG | LEU | 263 | −21.013 | 15.565 | 3.061 |
| ATOM | 2876 | HD13 | LEU | 263 | −22.791 | 16.258 | 1.415 |
| ATOM | 2877 | HD12 | LEU | 263 | −21.738 | 17.625 | 1.854 |
| ATOM | 2878 | HD11 | LEU | 263 | −23.37 | 17.494 | 2.57 |
| ATOM | 2879 | HD23 | LEU | 263 | −22.35 | 17.451 | 5.103 |
| ATOM | 2880 | HD22 | LEU | 263 | −20.904 | 17.907 | 4.159 |
| ATOM | 2881 | HD21 | LEU | 263 | −20.812 | 16.545 | 5.278 |
| ATOM | 2882 | H | LEU | 263 | −24.299 | 12.971 | 4.689 |
| ATOM | 2883 | HA | LEU | 263 | −21.523 | 13.744 | 4.833 |
| ATOM | 2884 | N | HIS | 264 | −23.81 | 15.149 | 6.883 |
| ATOM | 2885 | CA | HIS | 264 | −23.9 | 15.805 | 8.185 |
| ATOM | 2886 | C | HIS | 264 | −23.127 | 15.002 | 9.201 |
| ATOM | 2887 | O | HIS | 264 | −22.53 | 15.6 | 10.08 |
| ATOM | 2888 | CB | HIS | 264 | −25.364 | 15.962 | 8.669 |
| ATOM | 2889 | CG | HIS | 264 | −25.45 | 16.816 | 9.909 |
| ATOM | 2890 | ND1 | HIS | 264 | −24.947 | 16.49 | 11.078 |
| ATOM | 2891 | CD2 | HIS | 264 | −26.046 | 18.023 | 9.986 |
| ATOM | 2892 | CE1 | HIS | 264 | −25.169 | 17.44 | 11.929 |
| ATOM | 2893 | NE2 | HIS | 264 | −25.806 | 18.357 | 11.362 |
| ATOM | 2894 | HB2 | HIS | 264 | −25.923 | 16.464 | 7.868 |
| ATOM | 2895 | HB1 | HIS | 264 | −25.823 | 14.979 | 8.856 |
| ATOM | 2896 | HD1 | HIS | 264 | −24.447 | 15.615 | 11.29 |
| ATOM | 2897 | HD2 | HIS | 264 | −26.566 | 18.596 | 9.22 |
| ATOM | 2898 | HE1 | HIS | 264 | −24.87 | 17.468 | 12.976 |
| ATOM | 2899 | H | HIS | 264 | −24.655 | 14.939 | 6.387 |
| ATOM | 2900 | HA | HIS | 264 | −23.455 | 16.809 | 8.092 |
| ATOM | 2901 | N | LEU | 265 | −23.121 | 13.654 | 9.117 |
| ATOM | 2902 | CA | LEU | 265 | −22.331 | 12.879 | 10.069 |
| ATOM | 2903 | C | LEU | 265 | −20.859 | 13.157 | 9.857 |
| ATOM | 2904 | O | LEU | 265 | −20.216 | 13.6 | 10.797 |
| ATOM | 2905 | CB | LEU | 265 | −22.654 | 11.363 | 9.966 |
| ATOM | 2906 | CG | LEU | 265 | −21.987 | 10.444 | 11.033 |
| ATOM | 2907 | CD1 | LEU | 265 | −20.453 | 10.303 | 10.855 |
| ATOM | 2908 | CD2 | LEU | 265 | −22.334 | 10.838 | 12.495 |
| ATOM | 2909 | HB2 | LEU | 265 | −23.748 | 11.259 | 10.049 |
| ATOM | 2910 | HB1 | LEU | 265 | −22.364 | 10.997 | 8.972 |
| ATOM | 2911 | HG | LEU | 265 | −22.397 | 9.435 | 10.856 |
| ATOM | 2912 | HD13 | LEU | 265 | −20.181 | 10.115 | 9.805 |
| ATOM | 2913 | HD12 | LEU | 265 | −20.131 | 9.446 | 11.456 |
| ATOM | 2914 | HD11 | LEU | 265 | −19.902 | 11.179 | 11.218 |
| ATOM | 2915 | HD23 | LEU | 265 | −21.82 | 11.765 | 12.79 |
| ATOM | 2916 | HD22 | LEU | 265 | −22.015 | 10.042 | 13.186 |
| ATOM | 2917 | HD21 | LEU | 265 | −23.419 | 10.984 | 12.614 |
| ATOM | 2918 | H | LEU | 265 | −23.647 | 13.173 | 8.41 |
| ATOM | 2919 | HA | LEU | 265 | −22.628 | 13.21 | 11.076 |
| ATOM | 2920 | N | ILE | 266 | −20.283 | 12.905 | 8.654 |
| ATOM | 2921 | CA | ILE | 266 | −18.833 | 13.078 | 8.507 |
| ATOM | 2922 | C | ILE | 266 | −18.453 | 14.499 | 8.869 |
| ATOM | 2923 | O | ILE | 266 | −17.375 | 14.72 | 9.397 |
| ATOM | 2924 | CB | ILE | 266 | −18.25 | 12.663 | 7.117 |
| ATOM | 2925 | CG1 | ILE | 266 | −16.722 | 12.34 | 7.172 |
| ATOM | 2926 | CG2 | ILE | 266 | −18.486 | 13.775 | 6.057 |
| ATOM | 2927 | CD1 | ILE | 266 | −16.379 | 10.965 | 7.811 |
| ATOM | 2928 | HB | ILE | 266 | −18.774 | 11.752 | 6.781 |
| ATOM | 2929 | HG23 | ILE | 266 | −19.54 | 14.062 | 6.041 |
| ATOM | 2930 | HG22 | ILE | 266 | −18.218 | 13.419 | 5.05 |
| ATOM | 2931 | HG21 | ILE | 266 | −17.884 | 14.67 | 6.279 |
| ATOM | 2932 | HG12 | ILE | 266 | −16.191 | 13.141 | 7.711 |
| ATOM | 2933 | HG11 | ILE | 266 | −16.32 | 12.309 | 6.145 |
| ATOM | 2934 | HD13 | ILE | 266 | −16.711 | 10.886 | 8.855 |
| ATOM | 2935 | HD12 | ILE | 266 | −15.288 | 10.811 | 7.793 |
| ATOM | 2936 | HD11 | ILE | 266 | −16.845 | 10.157 | 7.228 |
| ATOM | 2937 | H | ILE | 266 | −20.828 | 12.593 | 7.87 |
| ATOM | 2938 | HA | ILE | 266 | −18.397 | 12.409 | 9.26 |
| ATOM | 2939 | N | PHE | 267 | −19.33 | 15.494 | 8.609 |
| ATOM | 2940 | CA | PHE | 267 | −19.047 | 16.842 | 9.095 |
| ATOM | 2941 | C | PHE | 267 | −19.101 | 16.84 | 10.607 |
| ATOM | 2942 | O | PHE | 267 | −18.134 | 17.242 | 11.233 |
| ATOM | 2943 | CB | PHE | 267 | −20.055 | 17.882 | 8.535 |
| ATOM | 2944 | CG | PHE | 267 | −19.938 | 19.209 | 9.299 |
| ATOM | 2945 | CD1 | PHE | 267 | −18.795 | 20.003 | 9.15 |
| ATOM | 2946 | CD2 | PHE | 267 | −20.964 | 19.635 | 10.151 |
| ATOM | 2947 | CE1 | PHE | 267 | −18.685 | 21.218 | 9.831 |
| ATOM | 2948 | CE2 | PHE | 267 | −20.866 | 20.86 | 10.816 |
| ATOM | 2949 | CZ | PHE | 267 | −19.726 | 21.654 | 10.656 |
| ATOM | 2950 | HB2 | PHE | 267 | −19.863 | 18.052 | 7.464 |
| ATOM | 2951 | HB1 | PHE | 267 | −21.08 | 17.497 | 8.627 |
| ATOM | 2952 | HD1 | PHE | 267 | −17.987 | 19.679 | 8.502 |
| ATOM | 2953 | HD2 | PHE | 267 | −21.845 | 19.02 | 10.305 |
| ATOM | 2954 | HE1 | PHE | 267 | −17.792 | 21.824 | 9.721 |
| ATOM | 2955 | HE2 | PHE | 267 | −21.675 | 21.19 | 11.46 |
| ATOM | 2956 | HZ | PHE | 267 | −19.645 | 22.603 | 11.174 |
| ATOM | 2957 | H | PHE | 267 | −20.2 | 15.316 | 8.144 |
| ATOM | 2958 | HA | PHE | 267 | −18.034 | 17.138 | 8.776 |
| ATOM | 2959 | N | TYR | 268 | −20.226 | 16.396 | 11.21 |
| ATOM | 2960 | CA | TYR | 268 | −20.375 | 16.46 | 12.663 |
| ATOM | 2961 | C | TYR | 268 | −19.181 | 15.87 | 13.371 |
| ATOM | 2962 | O | TYR | 268 | −18.662 | 16.505 | 14.275 |
| ATOM | 2963 | CB | TYR | 268 | −21.644 | 15.713 | 13.147 |
| ATOM | 2964 | CG | TYR | 268 | −21.773 | 15.771 | 14.676 |
| ATOM | 2965 | CD1 | TYR | 268 | −22.472 | 16.817 | 15.289 |
| ATOM | 2966 | CD2 | TYR | 268 | −21.2 | 14.773 | 15.471 |
| ATOM | 2967 | CE1 | TYR | 268 | −22.671 | 16.813 | 16.673 |
| ATOM | 2968 | CE2 | TYR | 268 | −21.36 | 14.793 | 16.859 |
| ATOM | 2969 | CZ | TYR | 268 | −22.119 | 15.803 | 17.464 |
| ATOM | 2970 | OH | TYR | 268 | −22.336 | 15.823 | 18.845 |
| ATOM | 2971 | HB1 | TYR | 268 | −22.531 | 16.177 | 12.694 |
| ATOM | 2972 | HB2 | TYR | 268 | −21.613 | 14.663 | 12.824 |
| ATOM | 2973 | HD1 | TYR | 268 | −22.866 | 17.634 | 14.694 |
| ATOM | 2974 | HD2 | TYR | 268 | −20.628 | 13.972 | 15.013 |
| ATOM | 2975 | HE1 | TYR | 268 | −23.258 | 17.596 | 17.14 |
| ATOM | 2976 | HE2 | TYR | 268 | −20.895 | 14.018 | 17.459 |
| ATOM | 2977 | HH | TYR | 268 | −22.049 | 15.037 | 19.295 |
| ATOM | 2978 | H | TYR | 268 | −20.985 | 16.039 | 10.666 |
| ATOM | 2979 | HA | TYR | 268 | −20.491 | 17.514 | 12.957 |
| ATOM | 2980 | N | ILE | 269 | −18.733 | 14.651 | 12.992 |
| ATOM | 2981 | CA | ILE | 269 | −17.61 | 14.054 | 13.716 |
| ATOM | 2982 | C | ILE | 269 | −16.375 | 14.912 | 13.556 |
| ATOM | 2983 | O | ILE | 269 | −15.575 | 14.949 | 14.477 |
| ATOM | 2984 | CB | ILE | 269 | −17.317 | 12.577 | 13.297 |
| ATOM | 2985 | CG1 | ILE | 269 | −16.468 | 11.779 | 14.335 |
| ATOM | 2986 | CG2 | ILE | 269 | −16.598 | 12.531 | 11.92 |
| ATOM | 2987 | CD1 | ILE | 269 | −17.158 | 11.59 | 15.715 |
| ATOM | 2988 | HB | ILE | 269 | −18.286 | 12.057 | 13.2 |
| ATOM | 2989 | HG23 | ILE | 269 | −17.183 | 13.093 | 11.186 |
| ATOM | 2990 | HG22 | ILE | 269 | −16.492 | 11.501 | 11.552 |
| ATOM | 2991 | HG21 | ILE | 269 | −15.59 | 12.971 | 11.975 |
| ATOM | 2992 | HG12 | ILE | 269 | −15.488 | 12.261 | 14.481 |
| ATOM | 2993 | HG11 | ILE | 269 | −16.289 | 10.771 | 13.923 |
| ATOM | 2994 | HD13 | ILE | 269 | −17.155 | 12.517 | 16.306 |
| ATOM | 2995 | HD12 | ILE | 269 | −16.627 | 10.826 | 16.304 |
| ATOM | 2996 | HD11 | ILE | 269 | −18.199 | 11.259 | 15.578 |
| ATOM | 2997 | H | ILE | 269 | −19.162 | 14.158 | 12.23 |
| ATOM | 2998 | HA | ILE | 269 | −17.901 | 14.069 | 14.776 |
| ATOM | 2999 | N | SER | 270 | −16.18 | 15.6 | 12.404 |
| ATOM | 3000 | CA | SER | 270 | −14.968 | 16.396 | 12.23 |
| ATOM | 3001 | C | SER | 270 | −15.128 | 17.734 | 12.913 |
| ATOM | 3002 | O | SER | 270 | −14.449 | 17.951 | 13.904 |
| ATOM | 3003 | CB | SER | 270 | −14.614 | 16.593 | 10.732 |
| ATOM | 3004 | OG | SER | 270 | −15.618 | 17.396 | 10.086 |
| ATOM | 3005 | HB2 | SER | 270 | −13.633 | 17.092 | 10.651 |
| ATOM | 3006 | HB1 | SER | 270 | −14.551 | 15.602 | 10.25 |
| ATOM | 3007 | HG | SER | 270 | −15.444 | 17.528 | 9.159 |
| ATOM | 3008 | H | SER | 270 | −16.855 | 15.61 | 11.662 |
| ATOM | 3009 | HA | SER | 270 | −14.113 | 15.862 | 12.682 |
| ATOM | 3010 | N | CYS | 271 | −16.001 | 18.634 | 12.397 |
| ATOM | 3011 | CA | CYS | 271 | −16.184 | 19.958 | 13.004 |
| ATOM | 3012 | C | CYS | 271 | −14.931 | 20.469 | 13.689 |
| ATOM | 3013 | O | CYS | 271 | −14.958 | 20.624 | 14.9 |
| ATOM | 3014 | CB | CYS | 271 | −17.372 | 19.868 | 13.999 |
| ATOM | 3015 | SG | CYS | 271 | −18.984 | 19.926 | 13.16 |
| ATOM | 3016 | HG | CYS | 271 | −18.808 | 19.242 | 12.447 |
| ATOM | 3017 | HB2 | CYS | 271 | −17.394 | 20.726 | 14.685 |
| ATOM | 3018 | H | CYS | 271 | −16.564 | 18.382 | 11.602 |
| ATOM | 3019 | HA | CYS | 271 | −16.443 | 20.705 | 12.236 |
| ATOM | 3020 | HB1 | CYS | 271 | −17.276 | 18.94 | 14.581 |
| ATOM | 3021 | LPG2 | CYS | 271 | −19.115 | 20.54 | 12.9 |
| ATOM | 3022 | LPG1 | CYS | 271 | −19.493 | 19.742 | 13.571 |
| ATOM | 3023 | N | PRO | 272 | −13.807 | 20.75 | 12.984 |
| ATOM | 3024 | CA | PRO | 272 | −12.596 | 21.169 | 13.681 |
| ATOM | 3025 | C | PRO | 272 | −12.803 | 22.276 | 14.69 |
| ATOM | 3026 | O | PRO | 272 | −12.167 | 22.258 | 15.731 |
| ATOM | 3027 | CB | PRO | 272 | −11.746 | 21.634 | 12.474 |
| ATOM | 3028 | CG | PRO | 272 | −12.217 | 20.706 | 11.33 |
| ATOM | 3029 | CD | PRO | 272 | −13.749 | 20.639 | 11.532 |
| ATOM | 3030 | HA | PRO | 272 | −12.146 | 20.284 | 14.161 |
| ATOM | 3031 | HB2 | PRO | 272 | −12.001 | 22.674 | 12.209 |
| ATOM | 3032 | HB1 | PRO | 272 | −10.663 | 21.572 | 12.667 |
| ATOM | 3033 | HG1 | PRO | 272 | −11.93 | 21.068 | 10.33 |
| ATOM | 3034 | HG2 | PRO | 272 | −11.783 | 19.703 | 11.486 |
| ATOM | 3035 | HD1 | PRO | 272 | −14.16 | 19.708 | 11.116 |
| ATOM | 3036 | HD2 | PRO | 272 | −14.246 | 21.508 | 11.073 |
| ATOM | 3037 | N | GLN | 273 | −13.693 | 23.252 | 14.405 |
| ATOM | 3038 | CA | GLN | 273 | −13.964 | 24.3 | 15.392 |
| ATOM | 3039 | C | GLN | 273 | −14.61 | 23.718 | 16.633 |
| ATOM | 3040 | O | GLN | 273 | −14.292 | 24.21 | 17.703 |
| ATOM | 3041 | CB | GLN | 273 | −14.881 | 25.394 | 14.782 |
| ATOM | 3042 | CG | GLN | 273 | −15.195 | 26.538 | 15.785 |
| ATOM | 3043 | CD | GLN | 273 | −16.036 | 27.631 | 15.171 |
| ATOM | 3044 | OE1 | GLN | 273 | −16.346 | 27.573 | 13.992 |
| ATOM | 3045 | NE2 | GLN | 273 | −16.43 | 28.658 | 15.952 |
| ATOM | 3046 | H | GLN | 273 | −14.196 | 23.247 | 13.539 |
| ATOM | 3047 | HA | GLN | 273 | −13.006 | 24.769 | 15.674 |
| ATOM | 3048 | HB2 | GLN | 273 | −14.381 | 25.817 | 13.895 |
| ATOM | 3049 | HB1 | GLN | 273 | −15.819 | 24.92 | 14.455 |
| ATOM | 3050 | HG2 | GLN | 273 | −14.254 | 26.986 | 16.139 |
| ATOM | 3051 | HG1 | GLN | 273 | −15.74 | 26.136 | 16.652 |
| ATOM | 3052 | HE21 | GLN | 273 | −16.174 | 28.706 | 16.918 |
| ATOM | 3053 | HE22 | GLN | 273 | −16.987 | 29.388 | 15.555 |
| ATOM | 3054 | N | ASN | 274 | −15.499 | 22.703 | 16.491 |
| ATOM | 3055 | CA | ASN | 274 | −16.151 | 22.006 | 17.611 |
| ATOM | 3056 | C | ASN | 274 | −17.643 | 21.973 | 17.311 |
| ATOM | 3057 | O | ASN | 274 | −18.155 | 23.059 | 17.092 |
| ATOM | 3058 | CB | ASN | 274 | −15.921 | 22.556 | 19.045 |
| ATOM | 3059 | CG | ASN | 274 | −16.565 | 21.665 | 20.079 |
| ATOM | 3060 | OD1 | ASN | 274 | −16.098 | 20.552 | 20.251 |
| ATOM | 3061 | ND2 | ASN | 274 | −17.624 | 22.096 | 20.794 |
| ATOM | 3062 | H | ASN | 274 | −15.706 | 22.354 | 15.575 |
| ATOM | 3063 | HA | ASN | 274 | −15.71 | 20.999 | 17.615 |
| ATOM | 3064 | HB2 | ASN | 274 | −14.848 | 22.553 | 19.292 |
| ATOM | 3065 | HB1 | ASN | 274 | −16.297 | 23.589 | 19.109 |
| ATOM | 3066 | HD21 | ASN | 274 | −17.995 | 23.016 | 20.679 |
| ATOM | 3067 | HD22 | ASN | 274 | −18.033 | 21.479 | 21.466 |
| ATOM | 3068 | N | PRO | 275 | −18.399 | 20.839 | 17.265 |
| ATOM | 3069 | CA | PRO | 275 | −19.799 | 20.896 | 16.845 |
| ATOM | 3070 | C | PRO | 275 | −20.653 | 22.021 | 17.376 |
| ATOM | 3071 | O | PRO | 275 | −21.452 | 22.562 | 16.626 |
| ATOM | 3072 | CB | PRO | 275 | −20.29 | 19.541 | 17.41 |
| ATOM | 3073 | CG | PRO | 275 | −19.074 | 18.613 | 17.214 |
| ATOM | 3074 | CD | PRO | 275 | −17.873 | 19.516 | 17.593 |
| ATOM | 3075 | HA | PRO | 275 | −19.837 | 20.89 | 15.745 |
| ATOM | 3076 | HB2 | PRO | 275 | −20.5 | 19.625 | 18.489 |
| ATOM | 3077 | HB1 | PRO | 275 | −21.191 | 19.168 | 16.902 |
| ATOM | 3078 | HG1 | PRO | 275 | −19.132 | 17.689 | 17.813 |
| ATOM | 3079 | HG2 | PRO | 275 | −19.028 | 18.337 | 16.149 |
| ATOM | 3080 | HD1 | PRO | 275 | −16.981 | 19.21 | 17.026 |
| ATOM | 3081 | HD2 | PRO | 275 | −17.682 | 19.445 | 18.674 |
| ATOM | 3082 | N | TYR | 276 | −20.514 | 22.382 | 18.669 |
| ATOM | 3083 | CA | TYR | 276 | −21.409 | 23.384 | 19.245 |
| ATOM | 3084 | C | TYR | 276 | −21.428 | 24.654 | 18.426 |
| ATOM | 3085 | O | TYR | 276 | −22.442 | 25.333 | 18.452 |
| ATOM | 3086 | CB | TYR | 276 | −21.031 | 23.767 | 20.702 |
| ATOM | 3087 | CG | TYR | 276 | −20.837 | 22.585 | 21.67 |
| ATOM | 3088 | CD1 | TYR | 276 | −20.105 | 22.802 | 22.844 |
| ATOM | 3089 | CD2 | TYR | 276 | −21.355 | 21.305 | 21.429 |
| ATOM | 3090 | CE1 | TYR | 276 | −19.844 | 21.756 | 23.733 |
| ATOM | 3091 | CE2 | TYR | 276 | −21.084 | 20.254 | 22.31 |
| ATOM | 3092 | CZ | TYR | 276 | −20.323 | 20.469 | 23.462 |
| ATOM | 3093 | OH | TYR | 276 | −20.059 | 19.395 | 24.317 |
| ATOM | 3094 | H | TYR | 276 | −19.823 | 21.945 | 19.244 |
| ATOM | 3095 | HA | TYR | 276 | −22.437 | 22.986 | 19.235 |
| ATOM | 3096 | HB1 | TYR | 276 | −20.092 | 24.34 | 20.658 |
| ATOM | 3097 | HB2 | TYR | 276 | −21.809 | 24.422 | 21.125 |
| ATOM | 3098 | HD1 | TYR | 276 | −19.73 | 23.795 | 23.072 |
| ATOM | 3099 | HD2 | TYR | 276 | −21.973 | 21.101 | 20.563 |
| ATOM | 3100 | HE1 | TYR | 276 | −19.267 | 21.953 | 24.631 |
| ATOM | 3101 | HE2 | TYR | 276 | −21.467 | 19.26 | 22.102 |
| ATOM | 3102 | HH | TYR | 276 | −19.517 | 19.615 | 25.067 |
| ATOM | 3103 | N | CYS | 277 | −20.342 | 25.012 | 17.703 |
| ATOM | 3104 | CA | CYS | 277 | −20.393 | 26.248 | 16.926 |
| ATOM | 3105 | C | CYS | 277 | −21.577 | 26.246 | 15.984 |
| ATOM | 3106 | O | CYS | 277 | −22.225 | 27.276 | 15.891 |
| ATOM | 3107 | CB | CYS | 277 | −19.091 | 26.527 | 16.129 |
| ATOM | 3108 | S1 | CYS | 277 | −18.761 | 25.19 | 14.934 |
| ATOM | 3109 | H11 | CYS | 277 | −18.131 | 25.684 | 14.325 |
| ATOM | 3110 | H | CYS | 277 | −19.504 | 24.46 | 17.687 |
| ATOM | 3111 | HA | CYS | 277 | −20.524 | 27.08 | 17.64 |
| ATOM | 3112 | HB2 | CYS | 277 | −19.188 | 27.485 | 15.592 |
| ATOM | 3113 | HB1 | CYS | 277 | −18.241 | 26.596 | 16.824 |
| ATOM | 3114 | N | VAL | 278 | −21.883 | 25.129 | 15.278 |
| ATOM | 3115 | CA | VAL | 278 | −22.997 | 25.143 | 14.326 |
| ATOM | 3116 | C | VAL | 278 | −23.751 | 23.83 | 14.231 |
| ATOM | 3117 | O | VAL | 278 | −24.411 | 23.635 | 13.223 |
| ATOM | 3118 | CB | VAL | 278 | −22.515 | 25.595 | 12.91 |
| ATOM | 3119 | CG1 | VAL | 278 | −21.866 | 27.007 | 12.925 |
| ATOM | 3120 | CG2 | VAL | 278 | −21.531 | 24.564 | 12.292 |
| ATOM | 3121 | H | VAL | 278 | −21.338 | 24.293 | 15.369 |
| ATOM | 3122 | HA | VAL | 278 | −23.771 | 25.846 | 14.672 |
| ATOM | 3123 | HB | VAL | 278 | −23.39 | 25.657 | 12.24 |
| ATOM | 3124 | HG23 | VAL | 278 | −20.626 | 24.471 | 12.907 |
| ATOM | 3125 | HG22 | VAL | 278 | −21.231 | 24.88 | 11.281 |
| ATOM | 3126 | HG21 | VAL | 278 | −22 | 23.573 | 12.213 |
| ATOM | 3127 | HG13 | VAL | 278 | −22.556 | 27.744 | 13.364 |
| ATOM | 3128 | HG12 | VAL | 278 | −21.632 | 27.329 | 11.899 |
| ATOM | 3129 | HG11 | VAL | 278 | −20.93 | 27.004 | 13.501 |
| ATOM | 3130 | N | CYS | 279 | −23.724 | 22.904 | 15.219 |
| ATOM | 3131 | CA | CYS | 279 | −24.498 | 21.667 | 15.072 |
| ATOM | 3132 | C | CYS | 279 | −25.96 | 21.975 | 15.316 |
| ATOM | 3133 | O | CYS | 279 | −26.532 | 21.531 | 16.299 |
| ATOM | 3134 | CB | CYS | 279 | −23.941 | 20.504 | 15.941 |
| ATOM | 3135 | SG | CYS | 279 | −23.801 | 20.88 | 17.723 |
| ATOM | 3136 | HG | CYS | 279 | −24.777 | 20.99 | 17.946 |
| ATOM | 3137 | H | CYS | 279 | −23.22 | 23.059 | 16.071 |
| ATOM | 3138 | HA | CYS | 279 | −24.424 | 21.302 | 14.033 |
| ATOM | 3139 | HB2 | CYS | 279 | −22.924 | 20.276 | 15.588 |
| ATOM | 3140 | HB1 | CYS | 279 | −24.563 | 19.604 | 15.814 |
| ATOM | 3141 | LPG2 | CYS | 279 | −23.45 | 21.45 | 17.835 |
| ATOM | 3142 | LPG1 | CYS | 279 | −23.519 | 20.368 | 18.069 |
| ATOM | 3143 | N | PHE | 280 | −26.601 | 22.748 | 14.41 |
| ATOM | 3144 | CA | PHE | 280 | −28.001 | 23.112 | 14.609 |
| ATOM | 3145 | C | PHE | 280 | −28.843 | 21.885 | 14.345 |
| ATOM | 3146 | O | PHE | 280 | −29.387 | 21.786 | 13.257 |
| ATOM | 3147 | CB | PHE | 280 | −28.397 | 24.292 | 13.679 |
| ATOM | 3148 | CG | PHE | 280 | −27.508 | 25.517 | 13.949 |
| ATOM | 3149 | CD1 | PHE | 280 | −26.582 | 25.959 | 12.996 |
| ATOM | 3150 | CD2 | PHE | 280 | −27.623 | 26.209 | 15.16 |
| ATOM | 3151 | CE1 | PHE | 280 | −25.806 | 27.096 | 13.238 |
| ATOM | 3152 | CE2 | PHE | 280 | −26.807 | 27.313 | 15.427 |
| ATOM | 3153 | CZ | PHE | 280 | −25.899 | 27.76 | 14.464 |
| ATOM | 3154 | H | PHE | 280 | −26.138 | 23.049 | 13.573 |
| ATOM | 3155 | HA | PHE | 280 | −28.158 | 23.438 | 15.65 |
| ATOM | 3156 | HB2 | PHE | 280 | −29.448 | 24.57 | 13.858 |
| ATOM | 3157 | HB1 | PHE | 280 | −28.307 | 23.98 | 12.626 |
| ATOM | 3158 | HD1 | PHE | 280 | −26.458 | 25.424 | 12.059 |
| ATOM | 3159 | HD2 | PHE | 280 | −28.349 | 25.896 | 15.902 |
| ATOM | 3160 | HE1 | PHE | 280 | −25.129 | 27.466 | 12.474 |
| ATOM | 3161 | HE2 | PHE | 280 | −26.879 | 27.824 | 16.382 |
| ATOM | 3162 | HZ | PHE | 280 | −25.268 | 28.62 | 14.667 |
| ATOM | 3163 | N | MET | 281 | −28.948 | 20.956 | 15.328 |
| ATOM | 3164 | CA | MET | 281 | −29.617 | 19.666 | 15.127 |
| ATOM | 3165 | C | MET | 281 | −30.707 | 19.631 | 14.079 |
| ATOM | 3166 | O | MET | 281 | −30.817 | 18.608 | 13.425 |
| ATOM | 3167 | CB | MET | 281 | −30.181 | 19.083 | 16.453 |
| ATOM | 3168 | CG | MET | 281 | −31.31 | 19.962 | 17.05 |
| ATOM | 3169 | SD | MET | 281 | −31.967 | 19.225 | 18.585 |
| ATOM | 3170 | CE | MET | 281 | −30.617 | 19.495 | 19.777 |
| ATOM | 3171 | HB2 | MET | 281 | −30.577 | 18.07 | 16.27 |
| ATOM | 3172 | HB1 | MET | 281 | −29.355 | 18.996 | 17.176 |
| ATOM | 3173 | HG2 | MET | 281 | −32.16 | 20.016 | 16.354 |
| ATOM | 3174 | HG1 | MET | 281 | −30.953 | 20.983 | 17.254 |
| ATOM | 3175 | HE3 | MET | 281 | −30.993 | 19.275 | 20.787 |
| ATOM | 3176 | HE2 | MET | 281 | −29.772 | 18.826 | 19.561 |
| ATOM | 3177 | HE1 | MET | 281 | −30.28 | 20.542 | 19.738 |
| ATOM | 3178 | H | MET | 281 | −28.456 | 21.092 | 16.193 |
| ATOM | 3179 | HA | MET | 281 | −28.826 | 18.979 | 14.778 |
| ATOM | 3180 | LPD2 | MET | 281 | −32.092 | 18.562 | 18.504 |
| ATOM | 3181 | LPD1 | MET | 281 | −32.535 | 19.539 | 18.783 |
| ATOM | 3182 | N | SER | 282 | −31.516 | 20.692 | 13.865 |
| ATOM | 3183 | CA | SER | 282 | −32.454 | 20.672 | 12.745 |
| ATOM | 3184 | C | SER | 282 | −31.805 | 20.127 | 11.489 |
| ATOM | 3185 | O | SER | 282 | −32.402 | 19.279 | 10.847 |
| ATOM | 3186 | CB | SER | 282 | −32.988 | 22.096 | 12.447 |
| ATOM | 3187 | OG | SER | 282 | −33.617 | 22.602 | 13.638 |
| ATOM | 3188 | HB2 | SER | 282 | −32.148 | 22.75 | 12.157 |
| ATOM | 3189 | HB1 | SER | 282 | −33.711 | 22.054 | 11.615 |
| ATOM | 3190 | HG | SER | 282 | −33.965 | 23.482 | 13.527 |
| ATOM | 3191 | H | SER | 282 | −31.46 | 21.515 | 14.432 |
| ATOM | 3192 | HA | SER | 282 | −33.309 | 20.036 | 13.027 |
| ATOM | 3193 | N | HIS | 283 | −30.585 | 20.575 | 11.11 |
| ATOM | 3194 | CA | HIS | 283 | −29.962 | 20.038 | 9.896 |
| ATOM | 3195 | C | HIS | 283 | −29.666 | 18.553 | 9.983 |
| ATOM | 3196 | O | HIS | 283 | −29.598 | 17.917 | 8.944 |
| ATOM | 3197 | CB | HIS | 283 | −28.743 | 20.874 | 9.406 |
| ATOM | 3198 | CG | HIS | 283 | −27.576 | 21.127 | 10.335 |
| ATOM | 3199 | ND1 | HIS | 283 | −26.58 | 21.904 | 9.967 |
| ATOM | 3200 | CD2 | HIS | 283 | −27.346 | 20.665 | 11.581 |
| ATOM | 3201 | CE1 | HIS | 283 | −25.702 | 21.982 | 10.915 |
| ATOM | 3202 | NE2 | HIS | 283 | −26.087 | 21.289 | 11.886 |
| ATOM | 3203 | HB2 | HIS | 283 | −28.345 | 20.41 | 8.49 |
| ATOM | 3204 | HB1 | HIS | 283 | −29.123 | 21.873 | 9.14 |
| ATOM | 3205 | HD1 | HIS | 283 | −26.505 | 22.383 | 9.057 |
| ATOM | 3206 | HD2 | HIS | 283 | −27.907 | 20.005 | 12.232 |
| ATOM | 3207 | HE1 | HIS | 283 | −24.773 | 22.552 | 10.891 |
| ATOM | 3208 | H | HIS | 283 | −30.092 | 21.254 | 11.659 |
| ATOM | 3209 | HA | HIS | 283 | −30.714 | 20.118 | 9.091 |
| ATOM | 3210 | N | PHE | 284 | −29.508 | 17.958 | 11.183 |
| ATOM | 3211 | CA | PHE | 284 | −29.337 | 16.507 | 11.297 |
| ATOM | 3212 | C | PHE | 284 | −30.708 | 15.872 | 11.305 |
| ATOM | 3213 | O | PHE | 284 | −30.951 | 14.95 | 10.541 |
| ATOM | 3214 | CB | PHE | 284 | −28.587 | 16.192 | 12.619 |
| ATOM | 3215 | CG | PHE | 284 | −28.317 | 14.7 | 12.86 |
| ATOM | 3216 | CD1 | PHE | 284 | −27.292 | 14.047 | 12.166 |
| ATOM | 3217 | CD2 | PHE | 284 | −29.066 | 13.982 | 13.801 |
| ATOM | 3218 | CE1 | PHE | 284 | −26.898 | 12.759 | 12.539 |
| ATOM | 3219 | CE2 | PHE | 284 | −28.669 | 12.697 | 14.183 |
| ATOM | 3220 | CZ | PHE | 284 | −27.563 | 12.097 | 13.575 |
| ATOM | 3221 | HB2 | PHE | 284 | −29.14 | 16.592 | 13.482 |
| ATOM | 3222 | HB1 | PHE | 284 | −27.62 | 16.713 | 12.593 |
| ATOM | 3223 | HD1 | PHE | 284 | −26.795 | 14.543 | 11.341 |
| ATOM | 3224 | HD2 | PHE | 284 | −29.951 | 14.423 | 14.248 |
| ATOM | 3225 | HE1 | PHE | 284 | −26.075 | 12.273 | 12.024 |
| ATOM | 3226 | HE2 | PHE | 284 | −29.219 | 12.166 | 14.953 |
| ATOM | 3227 | HZ | PHE | 284 | −27.227 | 11.118 | 13.902 |
| ATOM | 3228 | H | PHE | 284 | −29.602 | 18.487 | 12.022 |
| ATOM | 3229 | HA | PHE | 284 | −28.742 | 16.112 | 10.456 |
| ATOM | 3230 | N | ASN | 285 | −31.625 | 16.364 | 12.169 |
| ATOM | 3231 | CA | ASN | 285 | −32.958 | 15.781 | 12.259 |
| ATOM | 3232 | C | ASN | 285 | −33.633 | 15.809 | 10.908 |
| ATOM | 3233 | O | ASN | 285 | −34.15 | 14.787 | 10.493 |
| ATOM | 3234 | CB | ASN | 285 | −33.847 | 16.546 | 13.278 |
| ATOM | 3235 | CG | ASN | 285 | −33.214 | 16.614 | 14.647 |
| ATOM | 3236 | OD1 | ASN | 285 | −32.181 | 16.001 | 14.863 |
| ATOM | 3237 | ND2 | ASN | 285 | −33.807 | 17.357 | 15.604 |
| ATOM | 3238 | HB2 | ASN | 285 | −34.01 | 17.575 | 12.922 |
| ATOM | 3239 | HB1 | ASN | 285 | −34.825 | 16.048 | 13.363 |
| ATOM | 3240 | HD21 | ASN | 285 | −34.653 | 17.861 | 15.425 |
| ATOM | 3241 | HD22 | ASN | 285 | −33.391 | 17.404 | 16.513 |
| ATOM | 3242 | H | ASN | 285 | −31.407 | 17.124 | 12.78 |
| ATOM | 3243 | HA | ASN | 285 | −32.856 | 14.74 | 12.607 |
| ATOM | 3244 | N | LEU | 286 | −33.644 | 16.955 | 10.194 |
| ATOM | 3245 | CA | LEU | 286 | −34.325 | 17.008 | 8.898 |
| ATOM | 3246 | C | LEU | 286 | −33.828 | 15.894 | 8.005 |
| ATOM | 3247 | O | LEU | 286 | −34.629 | 15.217 | 7.378 |
| ATOM | 3248 | CB | LEU | 286 | −34.078 | 18.36 | 8.166 |
| ATOM | 3249 | CG | LEU | 286 | −34.83 | 19.573 | 8.798 |
| ATOM | 3250 | CD1 | LEU | 286 | −34.195 | 20.905 | 8.307 |
| ATOM | 3251 | CD2 | LEU | 286 | −36.347 | 19.566 | 8.454 |
| ATOM | 3252 | HB2 | LEU | 286 | −32.991 | 18.541 | 8.174 |
| ATOM | 3253 | HB1 | LEU | 286 | −34.386 | 18.274 | 7.111 |
| ATOM | 3254 | HG | LEU | 286 | −34.737 | 19.544 | 9.896 |
| ATOM | 3255 | HD13 | LEU | 286 | −33.137 | 20.966 | 8.607 |
| ATOM | 3256 | HD12 | LEU | 286 | −34.719 | 21.771 | 8.741 |
| ATOM | 3257 | HD11 | LEU | 286 | −34.252 | 20.974 | 7.209 |
| ATOM | 3258 | HD23 | LEU | 286 | −36.495 | 19.596 | 7.363 |
| ATOM | 3259 | HD22 | LEU | 286 | −36.841 | 20.447 | 8.893 |
| ATOM | 3260 | HD21 | LEU | 286 | −36.851 | 18.672 | 8.849 |
| ATOM | 3261 | H | LEU | 286 | −33.18 | 17.772 | 10.54 |
| ATOM | 3262 | HA | LEU | 286 | −35.401 | 16.848 | 9.061 |
| ATOM | 3263 | N | TYR | 287 | −32.498 | 15.682 | 7.927 |
| ATOM | 3264 | CA | TYR | 287 | −31.994 | 14.611 | 7.07 |
| ATOM | 3265 | C | TYR | 287 | −32.306 | 13.262 | 7.688 |
| ATOM | 3266 | O | TYR | 287 | −32.53 | 12.31 | 6.957 |
| ATOM | 3267 | CB | TYR | 287 | −30.481 | 14.804 | 6.802 |
| ATOM | 3268 | CG | TYR | 287 | −30.091 | 16.239 | 6.4 |
| ATOM | 3269 | CD1 | TYR | 287 | −30.988 | 17.158 | 5.841 |
| ATOM | 3270 | CD2 | TYR | 287 | −28.77 | 16.644 | 6.617 |
| ATOM | 3271 | CE1 | TYR | 287 | −30.585 | 18.472 | 5.583 |
| ATOM | 3272 | CE2 | TYR | 287 | −28.353 | 17.946 | 6.327 |
| ATOM | 3273 | CZ | TYR | 287 | −29.271 | 18.877 | 5.829 |
| ATOM | 3274 | OH | TYR | 287 | −28.903 | 20.201 | 5.572 |
| ATOM | 3275 | HB1 | TYR | 287 | −29.956 | 14.563 | 7.738 |
| ATOM | 3276 | HB2 | TYR | 287 | −30.14 | 14.115 | 6.015 |
| ATOM | 3277 | HD1 | TYR | 287 | −32.005 | 16.877 | 5.597 |
| ATOM | 3278 | HD2 | TYR | 287 | −28.061 | 15.937 | 7.025 |
| ATOM | 3279 | HE1 | TYR | 287 | −31.298 | 19.188 | 5.187 |
| ATOM | 3280 | HE2 | TYR | 287 | −27.318 | 18.226 | 6.496 |
| ATOM | 3281 | HH | TYR | 287 | −27.972 | 20.371 | 5.662 |
| ATOM | 3282 | H | TYR | 287 | −31.852 | 16.222 | 8.473 |
| ATOM | 3283 | HA | TYR | 287 | −32.507 | 14.655 | 6.097 |
| ATOM | 3284 | N | LEU | 288 | −32.351 | 13.153 | 9.035 |
| ATOM | 3285 | CA | LEU | 288 | −32.8 | 11.907 | 9.665 |
| ATOM | 3286 | C | LEU | 288 | −34.213 | 11.638 | 9.184 |
| ATOM | 3287 | O | LEU | 288 | −34.513 | 10.532 | 8.765 |
| ATOM | 3288 | CB | LEU | 288 | −32.762 | 12.016 | 11.22 |
| ATOM | 3289 | CG | LEU | 288 | −32.614 | 10.675 | 12.001 |
| ATOM | 3290 | CD1 | LEU | 288 | −32.499 | 10.989 | 13.521 |
| ATOM | 3291 | CD2 | LEU | 288 | −33.8 | 9.704 | 11.759 |
| ATOM | 3292 | HB2 | LEU | 288 | −31.89 | 12.625 | 11.5 |
| ATOM | 3293 | HB1 | LEU | 288 | −33.659 | 12.542 | 11.577 |
| ATOM | 3294 | HG | LEU | 288 | −31.684 | 10.168 | 11.687 |
| ATOM | 3295 | HD13 | LEU | 288 | −31.627 | 11.628 | 13.727 |
| ATOM | 3296 | HD12 | LEU | 288 | −32.381 | 10.06 | 14.093 |
| ATOM | 3297 | HD11 | LEU | 288 | −33.404 | 11.503 | 13.879 |
| ATOM | 3298 | HD23 | LEU | 288 | −34.755 | 10.222 | 11.935 |
| ATOM | 3299 | HD22 | LEU | 288 | −33.738 | 8.84 | 12.439 |
| ATOM | 3300 | HD21 | LEU | 288 | −33.78 | 9.313 | 10.733 |
| ATOM | 3301 | H | LEU | 288 | −32.125 | 13.938 | 9.613 |
| ATOM | 3302 | HA | LEU | 288 | −32.126 | 11.098 | 9.344 |
| ATOM | 3303 | N | ILE | 289 | −35.11 | 12.652 | 9.217 |
| ATOM | 3304 | CA | ILE | 289 | −36.47 | 12.441 | 8.723 |
| ATOM | 3305 | C | ILE | 289 | −36.349 | 11.988 | 7.286 |
| ATOM | 3306 | O | ILE | 289 | −36.954 | 10.99 | 6.924 |
| ATOM | 3307 | CB | ILE | 289 | −37.408 | 13.69 | 8.839 |
| ATOM | 3308 | CG1 | ILE | 289 | −38.055 | 13.84 | 10.252 |
| ATOM | 3309 | CG2 | ILE | 289 | −38.557 | 13.628 | 7.788 |
| ATOM | 3310 | CD1 | ILE | 289 | −37.038 | 14.014 | 11.409 |
| ATOM | 3311 | HB | ILE | 289 | −36.828 | 14.601 | 8.62 |
| ATOM | 3312 | HG23 | ILE | 289 | −38.172 | 13.687 | 6.758 |
| ATOM | 3313 | HG22 | ILE | 289 | −39.253 | 14.471 | 7.92 |
| ATOM | 3314 | HG21 | ILE | 289 | −39.116 | 12.686 | 7.901 |
| ATOM | 3315 | HG12 | ILE | 289 | −38.695 | 12.967 | 10.448 |
| ATOM | 3316 | HG11 | ILE | 289 | −38.71 | 14.729 | 10.265 |
| ATOM | 3317 | HD13 | ILE | 289 | −36.324 | 13.18 | 11.455 |
| ATOM | 3318 | HD12 | ILE | 289 | −37.568 | 14.056 | 12.372 |
| ATOM | 3319 | HD11 | ILE | 289 | −36.489 | 14.959 | 11.281 |
| ATOM | 3320 | H | ILE | 289 | −34.842 | 13.564 | 9.527 |
| ATOM | 3321 | HA | ILE | 289 | −36.92 | 11.614 | 9.29 |
| ATOM | 3322 | N | LEU | 290 | −35.578 | 12.718 | 6.448 |
| ATOM | 3323 | CA | LEU | 290 | −35.505 | 12.346 | 5.038 |
| ATOM | 3324 | C | LEU | 290 | −35.101 | 10.9 | 4.888 |
| ATOM | 3325 | O | LEU | 290 | −35.69 | 10.239 | 4.046 |
| ATOM | 3326 | CB | LEU | 290 | −34.503 | 13.215 | 4.23 |
| ATOM | 3327 | CG | LEU | 290 | −34.426 | 12.865 | 2.712 |
| ATOM | 3328 | CD1 | LEU | 290 | −35.769 | 13.103 | 1.967 |
| ATOM | 3329 | CD2 | LEU | 290 | −33.298 | 13.698 | 2.043 |
| ATOM | 3330 | HB2 | LEU | 290 | −34.764 | 14.278 | 4.352 |
| ATOM | 3331 | HB1 | LEU | 290 | −33.505 | 13.05 | 4.663 |
| ATOM | 3332 | HG | LEU | 290 | −34.158 | 11.801 | 2.603 |
| ATOM | 3333 | HD13 | LEU | 290 | −36.553 | 12.415 | 2.314 |
| ATOM | 3334 | HD12 | LEU | 290 | −35.641 | 12.931 | 0.887 |
| ATOM | 3335 | HD11 | LEU | 290 | −36.111 | 14.138 | 2.118 |
| ATOM | 3336 | HD23 | LEU | 290 | −33.496 | 14.775 | 2.154 |
| ATOM | 3337 | HD22 | LEU | 290 | −33.228 | 13.466 | 0.969 |
| ATOM | 3338 | HD21 | LEU | 290 | −32.328 | 13.467 | 2.51 |
| ATOM | 3339 | H | LEU | 290 | −35.089 | 13.534 | 6.768 |
| ATOM | 3340 | HA | LEU | 290 | −36.519 | 12.488 | 4.635 |
| ATOM | 3341 | N | ILE | 291 | −34.119 | 10.385 | 5.666 |
| ATOM | 3342 | CA | ILE | 291 | −33.716 | 8.99 | 5.483 |
| ATOM | 3343 | C | ILE | 291 | −34.941 | 8.117 | 5.363 |
| ATOM | 3344 | O | ILE | 291 | −35.027 | 7.347 | 4.421 |
| ATOM | 3345 | CB | ILE | 291 | −32.799 | 8.401 | 6.603 |
| ATOM | 3346 | CG1 | ILE | 291 | −31.328 | 8.868 | 6.392 |
| ATOM | 3347 | CG2 | ILE | 291 | −32.886 | 6.844 | 6.594 |
| ATOM | 3348 | CD1 | ILE | 291 | −30.391 | 8.598 | 7.602 |
| ATOM | 3349 | HB | ILE | 291 | −33.153 | 8.756 | 7.584 |
| ATOM | 3350 | HG23 | ILE | 291 | −33.87 | 6.484 | 6.931 |
| ATOM | 3351 | HG22 | ILE | 291 | −32.155 | 6.387 | 7.262 |
| ATOM | 3352 | HG21 | ILE | 291 | −32.7 | 6.467 | 5.577 |
| ATOM | 3353 | HG12 | ILE | 291 | −31.306 | 9.951 | 6.188 |
| ATOM | 3354 | HG11 | ILE | 291 | −30.93 | 8.356 | 5.502 |
| ATOM | 3355 | HD13 | ILE | 291 | −30.655 | 9.252 | 8.446 |
| ATOM | 3356 | HD12 | ILE | 291 | −29.36 | 8.806 | 7.295 |
| ATOM | 3357 | HD11 | ILE | 291 | −30.399 | 7.562 | 7.96 |
| ATOM | 3358 | H | ILE | 291 | −33.643 | 10.949 | 6.346 |
| ATOM | 3359 | HA | ILE | 291 | −33.166 | 8.93 | 4.533 |
| ATOM | 3360 | N | MET | 292 | −35.898 | 8.207 | 6.31 |
| ATOM | 3361 | CA | MET | 292 | −37.006 | 7.253 | 6.299 |
| ATOM | 3362 | C | MET | 292 | −37.866 | 7.348 | 5.056 |
| ATOM | 3363 | O | MET | 292 | −38.731 | 6.499 | 4.906 |
| ATOM | 3364 | CB | MET | 292 | −37.812 | 7.285 | 7.628 |
| ATOM | 3365 | CG | MET | 292 | −36.956 | 6.736 | 8.805 |
| ATOM | 3366 | SD | MET | 292 | −37.889 | 6.871 | 10.368 |
| ATOM | 3367 | CE | MET | 292 | −36.817 | 7.972 | 11.348 |
| ATOM | 3368 | HB2 | MET | 292 | −38.126 | 8.32 | 7.829 |
| ATOM | 3369 | HB1 | MET | 292 | −38.719 | 6.665 | 7.539 |
| ATOM | 3370 | HG2 | MET | 292 | −36.002 | 7.279 | 8.885 |
| ATOM | 3371 | HG1 | MET | 292 | −36.73 | 5.669 | 8.65 |
| ATOM | 3372 | HE3 | MET | 292 | −35.915 | 7.446 | 11.676 |
| ATOM | 3373 | HE2 | MET | 292 | −37.365 | 8.306 | 12.238 |
| ATOM | 3374 | HE1 | MET | 292 | −36.523 | 8.846 | 10.751 |
| ATOM | 3375 | H | MET | 292 | −35.847 | 8.91 | 7.025 |
| ATOM | 3376 | HA | MET | 292 | −36.573 | 6.241 | 6.229 |
| ATOM | 3377 | LPD2 | MET | 292 | −37.961 | 6.266 | 10.666 |
| ATOM | 3378 | LPD1 | MET | 292 | −38.501 | 7.148 | 10.267 |
| ATOM | 3379 | N | CYS | 293 | −37.666 | 8.3 | 4.114 |
| ATOM | 3380 | CA | CYS | 293 | −38.315 | 8.147 | 2.814 |
| ATOM | 3381 | C | CYS | 293 | −37.839 | 6.865 | 2.162 |
| ATOM | 3382 | O | CYS | 293 | −38.598 | 6.318 | 1.378 |
| ATOM | 3383 | CB | CYS | 293 | −38.108 | 9.353 | 1.857 |
| ATOM | 3384 | SG | CYS | 293 | −36.395 | 9.489 | 1.247 |
| ATOM | 3385 | HB2 | CYS | 293 | −38.771 | 9.256 | 0.982 |
| ATOM | 3386 | HB1 | CYS | 293 | −38.365 | 10.286 | 2.383 |
| ATOM | 3387 | H | CYS | 293 | −37.018 | 9.053 | 4.233 |
| ATOM | 3388 | HG | CYS | 293 | −36.339 | 8.624 | 0.733 |
| ATOM | 3389 | HA | CYS | 293 | −39.4 | 8.06 | 2.994 |
| ATOM | 3390 | LPG2 | CYS | 293 | −35.945 | 9.511 | 1.755 |
| ATOM | 3391 | LPG1 | CYS | 293 | −36.31 | 10.029 | 0.845 |
| ATOM | 3392 | N | ASN | 294 | −36.621 | 6.346 | 2.467 |
| ATOM | 3393 | CA | ASN | 294 | −36.222 | 5.033 | 1.949 |
| ATOM | 3394 | C | ASN | 294 | −37.375 | 4.071 | 2.099 |
| ATOM | 3395 | O | ASN | 294 | −37.663 | 3.312 | 1.187 |
| ATOM | 3396 | CB | ASN | 294 | −34.971 | 4.424 | 2.649 |
| ATOM | 3397 | CG | ASN | 294 | −35.207 | 3.995 | 4.08 |
| ATOM | 3398 | OD1 | ASN | 294 | −34.839 | 4.72 | 4.989 |
| ATOM | 3399 | ND2 | ASN | 294 | −35.814 | 2.817 | 4.339 |
| ATOM | 3400 | HB2 | ASN | 294 | −34.641 | 3.536 | 2.086 |
| ATOM | 3401 | HB1 | ASN | 294 | −34.147 | 5.154 | 2.622 |
| ATOM | 3402 | HD21 | ASN | 294 | −36.095 | 2.177 | 3.62 |
| ATOM | 3403 | HD22 | ASN | 294 | −35.994 | 2.557 | 5.288 |
| ATOM | 3404 | H | ASN | 294 | −35.981 | 6.827 | 3.069 |
| ATOM | 3405 | HA | ASN | 294 | −35.976 | 5.151 | 0.885 |
| ATOM | 3406 | N | SER | 295 | −38.045 | 4.119 | 3.271 |
| ATOM | 3407 | CA | SER | 295 | −39.128 | 3.186 | 3.549 |
| ATOM | 3408 | C | SER | 295 | −40.211 | 3.186 | 2.493 |
| ATOM | 3409 | O | SER | 295 | −40.818 | 2.147 | 2.284 |
| ATOM | 3410 | CB | SER | 295 | −39.75 | 3.572 | 4.914 |
| ATOM | 3411 | OG | SER | 295 | −38.694 | 3.858 | 5.849 |
| ATOM | 3412 | HB2 | SER | 295 | −40.385 | 4.465 | 4.785 |
| ATOM | 3413 | HB1 | SER | 295 | −40.362 | 2.743 | 5.291 |
| ATOM | 3414 | HG | SER | 295 | −39.026 | 4.164 | 6.687 |
| ATOM | 3415 | H | SER | 295 | −37.788 | 4.763 | 3.997 |
| ATOM | 3416 | HA | SER | 295 | −38.684 | 2.185 | 3.616 |
| ATOM | 3417 | N | ILE | 296 | −40.471 | 4.345 | 1.843 |
| ATOM | 3418 | CA | ILE | 296 | −41.522 | 4.436 | 0.826 |
| ATOM | 3419 | C | ILE | 296 | −40.941 | 4.437 | −0.576 |
| ATOM | 3420 | O | ILE | 296 | −41.57 | 3.881 | −1.465 |
| ATOM | 3421 | CB | ILE | 296 | −42.429 | 5.688 | 1.078 |
| ATOM | 3422 | CG1 | ILE | 296 | −41.842 | 7.01 | 0.493 |
| ATOM | 3423 | CG2 | ILE | 296 | −42.739 | 5.818 | 2.599 |
| ATOM | 3424 | CD1 | ILE | 296 | −42.794 | 8.232 | 0.609 |
| ATOM | 3425 | HB | ILE | 296 | −43.383 | 5.516 | 0.547 |
| ATOM | 3426 | HG23 | ILE | 296 | −43.13 | 4.865 | 2.984 |
| ATOM | 3427 | HG22 | ILE | 296 | −43.491 | 6.593 | 2.788 |
| ATOM | 3428 | HG21 | ILE | 296 | −41.842 | 6.085 | 3.175 |
| ATOM | 3429 | HG12 | ILE | 296 | −41.651 | 6.879 | −0.585 |
| ATOM | 3430 | HG11 | ILE | 296 | −40.892 | 7.258 | 0.986 |
| ATOM | 3431 | HD13 | ILE | 296 | −43.784 | 7.991 | 0.192 |
| ATOM | 3432 | HD12 | ILE | 296 | −42.378 | 9.083 | 0.047 |
| ATOM | 3433 | HD11 | ILE | 296 | −42.917 | 8.554 | 1.652 |
| ATOM | 3434 | H | ILE | 296 | −39.942 | 5.168 | 2.052 |
| ATOM | 3435 | HA | ILE | 296 | −42.186 | 3.555 | 0.898 |
| ATOM | 3436 | N | ILE | 297 | −39.767 | 5.063 | −0.84 |
| ATOM | 3437 | CA | ILE | 297 | −39.318 | 5.197 | −2.225 |
| ATOM | 3438 | C | ILE | 297 | −38.916 | 3.876 | −2.847 |
| ATOM | 3439 | O | ILE | 297 | −39.017 | 3.783 | −4.061 |
| ATOM | 3440 | CB | ILE | 297 | −38.332 | 6.377 | −2.515 |
| ATOM | 3441 | CG1 | ILE | 297 | −36.963 | 6.354 | −1.769 |
| ATOM | 3442 | CG2 | ILE | 297 | −39.029 | 7.74 | −2.222 |
| ATOM | 3443 | CD1 | ILE | 297 | −36.122 | 5.068 | −1.977 |
| ATOM | 3444 | HB | ILE | 297 | −38.112 | 6.358 | −3.596 |
| ATOM | 3445 | HG23 | ILE | 297 | −39.96 | 7.848 | −2.801 |
| ATOM | 3446 | HG22 | ILE | 297 | −38.371 | 8.581 | −2.489 |
| ATOM | 3447 | HG21 | ILE | 297 | −39.274 | 7.821 | −1.153 |
| ATOM | 3448 | HG12 | ILE | 297 | −37.115 | 6.525 | −0.696 |
| ATOM | 3449 | HG11 | ILE | 297 | −36.36 | 7.201 | −2.138 |
| ATOM | 3450 | HD13 | ILE | 297 | −36.528 | 4.244 | −1.38 |
| ATOM | 3451 | HD12 | ILE | 297 | −35.085 | 5.234 | −1.644 |
| ATOM | 3452 | HD11 | ILE | 297 | −36.102 | 4.765 | −3.033 |
| ATOM | 3453 | H | ILE | 297 | −39.203 | 5.466 | −0.12 |
| ATOM | 3454 | HA | ILE | 297 | −40.215 | 5.476 | −2.8 |
| ATOM | 3455 | N | ASP | 298 | −38.479 | 2.839 | −2.092 |
| ATOM | 3456 | CA | ASP | 298 | −38.113 | 1.579 | −2.747 |
| ATOM | 3457 | C | ASP | 298 | −39.376 | 0.892 | −3.243 |
| ATOM | 3458 | O | ASP | 298 | −39.465 | 0.692 | −4.447 |
| ATOM | 3459 | CB | ASP | 298 | −37.157 | 0.66 | −1.924 |
| ATOM | 3460 | CG | ASP | 298 | −35.703 | 0.898 | −2.248 |
| ATOM | 3461 | OD1 | ASP | 298 | −35.282 | 2.085 | −2.264 |
| ATOM | 3462 | OD2 | ASP | 298 | −34.969 | −0.098 | −2.498 |
| ATOM | 3463 | HB2 | ASP | 298 | −37.255 | 0.82 | −0.841 |
| ATOM | 3464 | HB1 | ASP | 298 | −37.39 | −0.394 | −2.142 |
| ATOM | 3465 | H | ASP | 298 | −38.43 | 2.903 | −1.093 |
| ATOM | 3466 | HA | ASP | 298 | −37.539 | 1.829 | −3.656 |
| ATOM | 3467 | N | PRO | 299 | −40.396 | 0.513 | −2.428 |
| ATOM | 3468 | CA | PRO | 299 | −41.613 | −0.039 | −3.009 |
| ATOM | 3469 | C | PRO | 299 | −42.226 | 0.821 | −4.086 |
| ATOM | 3470 | O | PRO | 299 | −42.71 | 0.277 | −5.065 |
| ATOM | 3471 | CB | PRO | 299 | −42.547 | −0.048 | −1.777 |
| ATOM | 3472 | CG | PRO | 299 | −41.569 | −0.24 | −0.603 |
| ATOM | 3473 | CD | PRO | 299 | −40.355 | 0.643 | −0.976 |
| ATOM | 3474 | HB2 | PRO | 299 | −43.317 | −0.834 | −1.832 |
| ATOM | 3475 | HB1 | PRO | 299 | −43.038 | 0.931 | −1.651 |
| ATOM | 3476 | HG1 | PRO | 299 | −42.022 | 0.037 | 0.361 |
| ATOM | 3477 | HG2 | PRO | 299 | −41.264 | −1.3 | −0.585 |
| ATOM | 3478 | HD1 | PRO | 299 | −40.528 | 1.683 | −0.667 |
| ATOM | 3479 | HD2 | PRO | 299 | −39.453 | 0.245 | −0.493 |
| ATOM | 3480 | HA | PRO | 299 | −41.416 | −1.065 | −3.36 |
| ATOM | 3481 | N | LEU | 300 | −42.233 | 2.163 | −3.918 |
| ATOM | 3482 | CA | LEU | 300 | −42.882 | 3.011 | −4.917 |
| ATOM | 3483 | C | LEU | 300 | −42.097 | 2.927 | −6.204 |
| ATOM | 3484 | O | LEU | 300 | −42.668 | 2.59 | −7.229 |
| ATOM | 3485 | CB | LEU | 300 | −43.034 | 4.466 | −4.385 |
| ATOM | 3486 | CG | LEU | 300 | −43.8 | 5.451 | −5.322 |
| ATOM | 3487 | CD1 | LEU | 300 | −44.354 | 6.642 | −4.487 |
| ATOM | 3488 | CD2 | LEU | 300 | −42.902 | 6.018 | −6.462 |
| ATOM | 3489 | HB2 | LEU | 300 | −43.58 | 4.379 | −3.431 |
| ATOM | 3490 | HB1 | LEU | 300 | −42.045 | 4.886 | −4.162 |
| ATOM | 3491 | HG | LEU | 300 | −44.663 | 4.926 | −5.766 |
| ATOM | 3492 | HD13 | LEU | 300 | −45.054 | 6.286 | −3.714 |
| ATOM | 3493 | HD12 | LEU | 300 | −44.896 | 7.352 | −5.131 |
| ATOM | 3494 | HD11 | LEU | 300 | −43.529 | 7.176 | −3.99 |
| ATOM | 3495 | HD23 | LEU | 300 | −42.039 | 6.554 | −6.039 |
| ATOM | 3496 | HD22 | LEU | 300 | −43.478 | 6.724 | −7.08 |
| ATOM | 3497 | HD21 | LEU | 300 | −42.524 | 5.236 | −7.13 |
| ATOM | 3498 | H | LEU | 300 | −41.807 | 2.588 | −3.113 |
| ATOM | 3499 | HA | LEU | 300 | −43.899 | 2.621 | −5.094 |
| ATOM | 3500 | N | ILE | 301 | −40.779 | 3.225 | −6.191 |
| ATOM | 3501 | CA | ILE | 301 | −40.021 | 3.178 | −7.444 |
| ATOM | 3502 | C | ILE | 301 | −40.151 | 1.795 | −8.033 |
| ATOM | 3503 | O | ILE | 301 | −40.337 | 1.683 | −9.234 |
| ATOM | 3504 | CB | ILE | 301 | −38.528 | 3.577 | −7.259 |
| ATOM | 3505 | CG1 | ILE | 301 | −38.429 | 5.102 | −6.949 |
| ATOM | 3506 | CG2 | ILE | 301 | −37.686 | 3.208 | −8.517 |
| ATOM | 3507 | CD1 | ILE | 301 | −37.08 | 5.501 | −6.298 |
| ATOM | 3508 | H | ILE | 301 | −40.311 | 3.446 | −5.332 |
| ATOM | 3509 | HB | ILE | 301 | −38.14 | 3.001 | −6.405 |
| ATOM | 3510 | HG12 | ILE | 301 | −39.223 | 5.402 | −6.246 |
| ATOM | 3511 | HG11 | ILE | 301 | −38.576 | 5.679 | −7.877 |
| ATOM | 3512 | HG23 | ILE | 301 | −37.666 | 2.118 | −8.667 |
| ATOM | 3513 | HG22 | ILE | 301 | −36.645 | 3.539 | −8.416 |
| ATOM | 3514 | HG21 | ILE | 301 | −38.113 | 3.677 | −9.416 |
| ATOM | 3515 | HD13 | ILE | 301 | −36.945 | 4.959 | −5.352 |
| ATOM | 3516 | HD12 | ILE | 301 | −37.089 | 6.581 | −6.09 |
| ATOM | 3517 | HD11 | ILE | 301 | −36.219 | 5.283 | −6.941 |
| ATOM | 3518 | HA | ILE | 301 | −40.476 | 3.886 | −8.157 |
| ATOM | 3519 | N | TYR | 302 | −40.066 | 0.724 | −7.215 |
| ATOM | 3520 | CA | TYR | 302 | −40.28 | −0.611 | −7.769 |
| ATOM | 3521 | C | TYR | 302 | −41.656 | −0.719 | −8.386 |
| ATOM | 3522 | O | TYR | 302 | −41.766 | −1.221 | −9.492 |
| ATOM | 3523 | CB | TYR | 302 | −40.161 | −1.719 | −6.691 |
| ATOM | 3524 | CG | TYR | 302 | −40.694 | −3.068 | −7.205 |
| ATOM | 3525 | CD1 | TYR | 302 | −40.172 | −3.648 | −8.368 |
| ATOM | 3526 | CD2 | TYR | 302 | −41.705 | −3.741 | −6.508 |
| ATOM | 3527 | CE1 | TYR | 302 | −40.63 | −4.894 | −8.808 |
| ATOM | 3528 | CE2 | TYR | 302 | −42.157 | −4.99 | −6.942 |
| ATOM | 3529 | CZ | TYR | 302 | −41.619 | −5.576 | −8.09 |
| ATOM | 3530 | OH | TYR | 302 | −42.075 | −6.831 | −8.496 |
| ATOM | 3531 | H | TYR | 302 | −39.883 | 0.832 | −6.233 |
| ATOM | 3532 | HB1 | TYR | 302 | −40.738 | −1.409 | −5.808 |
| ATOM | 3533 | HB2 | TYR | 302 | −39.11 | −1.827 | −6.379 |
| ATOM | 3534 | HD1 | TYR | 302 | −39.4 | −3.14 | −8.936 |
| ATOM | 3535 | HD2 | TYR | 302 | −42.145 | −3.303 | −5.62 |
| ATOM | 3536 | HE1 | TYR | 302 | −40.209 | −5.327 | −9.708 |
| ATOM | 3537 | HE2 | TYR | 302 | −42.931 | −5.513 | −6.389 |
| ATOM | 3538 | HH | TYR | 302 | −42.015 | −6.968 | −9.435 |
| ATOM | 3539 | HA | TYR | 302 | −39.523 | −0.777 | −8.554 |
| ATOM | 3540 | N | ALA | 303 | −42.723 | −0.271 | −7.693 |
| ATOM | 3541 | CA | ALA | 303 | −44.063 | −0.397 | −8.262 |
| ATOM | 3542 | C | ALA | 303 | −44.132 | 0.332 | −9.585 |
| ATOM | 3543 | O | ALA | 303 | −44.679 | −0.205 | −10.536 |
| ATOM | 3544 | CB | ALA | 303 | −45.146 | 0.175 | −7.31 |
| ATOM | 3545 | HB3 | ALA | 303 | −44.975 | 1.246 | −7.127 |
| ATOM | 3546 | HB2 | ALA | 303 | −45.121 | −0.356 | −6.346 |
| ATOM | 3547 | HB1 | ALA | 303 | −46.146 | 0.051 | −7.755 |
| ATOM | 3548 | H | ALA | 303 | −42.612 | 0.153 | −6.793 |
| ATOM | 3549 | HA | ALA | 303 | −44.273 | −1.468 | −8.427 |
| ATOM | 3550 | N | LEU | 304 | −43.578 | 1.561 | −9.668 |
| ATOM | 3551 | CA | LEU | 304 | −43.591 | 2.283 | −10.941 |
| ATOM | 3552 | C | LEU | 304 | −42.663 | 1.617 | −11.937 |
| ATOM | 3553 | O | LEU | 304 | −42.984 | 1.644 | −13.115 |
| ATOM | 3554 | CB | LEU | 304 | −43.199 | 3.782 | −10.777 |
| ATOM | 3555 | CG | LEU | 304 | −44.358 | 4.721 | −10.312 |
| ATOM | 3556 | CD1 | LEU | 304 | −45.376 | 5.02 | −11.451 |
| ATOM | 3557 | CD2 | LEU | 304 | −45.109 | 4.193 | −9.059 |
| ATOM | 3558 | HB2 | LEU | 304 | −42.352 | 3.85 | −10.075 |
| ATOM | 3559 | HB1 | LEU | 304 | −42.836 | 4.167 | −11.745 |
| ATOM | 3560 | HG | LEU | 304 | −43.898 | 5.689 | −10.04 |
| ATOM | 3561 | HD13 | LEU | 304 | −44.862 | 5.415 | −12.34 |
| ATOM | 3562 | HD12 | LEU | 304 | −46.103 | 5.778 | −11.117 |
| ATOM | 3563 | HD11 | LEU | 304 | −45.938 | 4.121 | −11.743 |
| ATOM | 3564 | HD23 | LEU | 304 | −45.626 | 3.246 | −9.272 |
| ATOM | 3565 | HD22 | LEU | 304 | −45.86 | 4.927 | −8.726 |
| ATOM | 3566 | HD21 | LEU | 304 | −44.409 | 4.034 | −8.23 |
| ATOM | 3567 | H | LEU | 304 | −43.119 | 1.964 | −8.871 |
| ATOM | 3568 | HA | LEU | 304 | −44.601 | 2.222 | −11.375 |
| ATOM | 3569 | N | ARG | 305 | −41.516 | 1.031 | −11.519 |
| ATOM | 3570 | CA | ARG | 305 | −40.582 | 0.436 | −12.475 |
| ATOM | 3571 | C | ARG | 305 | −40.191 | −0.947 | −12.018 |
| ATOM | 3572 | O | ARG | 305 | −39.023 | −1.299 | −11.937 |
| ATOM | 3573 | CB | ARG | 305 | −39.362 | 1.378 | −12.68 |
| ATOM | 3574 | CG | ARG | 305 | −39.837 | 2.832 | −12.968 |
| ATOM | 3575 | CD | ARG | 305 | −38.739 | 3.727 | −13.611 |
| ATOM | 3576 | NE | ARG | 305 | −38.524 | 3.397 | −15.027 |
| ATOM | 3577 | CZ | ARG | 305 | −37.827 | 4.141 | −15.859 |
| ATOM | 3578 | NH1 | ARG | 305 | −37.203 | 5.233 | −15.487 |
| ATOM | 3579 | NH2 | ARG | 305 | −37.753 | 3.781 | −17.12 |
| ATOM | 3580 | HB2 | ARG | 305 | −38.739 | 1.381 | −11.77 |
| ATOM | 3581 | HB1 | ARG | 305 | −38.749 | 0.99 | −13.508 |
| ATOM | 3582 | HG2 | ARG | 305 | −40.171 | 3.277 | −12.014 |
| ATOM | 3583 | HG1 | ARG | 305 | −40.701 | 2.827 | −13.652 |
| ATOM | 3584 | HD2 | ARG | 305 | −37.803 | 3.63 | −13.036 |
| ATOM | 3585 | HD1 | ARG | 305 | −39.115 | 4.761 | −13.53 |
| ATOM | 3586 | HE | ARG | 305 | −38.986 | 2.548 | −15.388 |
| ATOM | 3587 | HH21 | ARG | 305 | −38.224 | 2.931 | −17.463 |
| ATOM | 3588 | HH22 | ARG | 305 | −37.228 | 4.343 | −17.807 |
| ATOM | 3589 | HH11 | ARG | 305 | −37.214 | 5.555 | −14.514 |
| ATOM | 3590 | HH12 | ARG | 305 | −36.672 | 5.8 | −16.164 |
| ATOM | 3591 | H | ARG | 305 | −41.259 | 1.026 | −10.551 |
| ATOM | 3592 | HA | ARG | 305 | −41.055 | 0.281 | −13.459 |
| ATOM | 3593 | H13 | ARG | 305 | −40.975 | −1.661 | −11.771 |

The data shown in Table 1 are expressed based on the Protein Data Bank (PDB) format: The PDB format is a format containing coordinates (X, Y, Z,), etc. of individual atoms constituting a protein, and is one of the standard formats in handling coordinates of biopolymers. In Table 1, the “ATOM” appearing in the utmost left column (1st column) denotes each atom of the atomic coordinates. The numbers (1, 2, 3, . . . 3593) appearing in the next column (2nd column) are serial numbers of individual atoms. Subsequently, in the left to right direction in this Table, there are denoted the type of each atom and its position in the amino acid to which it belongs (e.g., “CB”, “CG”, “SD”) (in the 3rd column); the amino acid residue to which each atom belongs (three-letter abbreviations for amino acids, e.g. “MET”, “ASN”) (in the 4th column); the sequence number of the residue counted from the N-terminal (in the 5th column); X-coordinate (in angstrom unit) (in the 6th column); Y-coordinate (in angstrom unit) (in the 7th column); Z-coordinate (in angstrom unit)(in the 8th column).

In addition to the above-described atomic coordinates, the present invention also includes derivatives therefrom, i.e., derivatives from the above-mentioned atomic coordinates (related receptors, etc.) obtained by techniques such as homology modeling on computer. The term “derivatives” means proteins of the GPCR family that have 15% or more, preferably 30% or more, homology to native human MC4-R in the amino acid sequence; or atomic coordinates (derived coordinates) in which a part of the amino acid sequence of native human MC4-R (preferably one or several (e.g., one to ten) amino acids) is deleted, substituted or added and which exhibit a three-dimensional structure of a protein having activity functionally equivalent to native human MC4-R (i.e., the signal transduction activity of GPCRs). Further, in the case of a GPCR family protein that is too low in homology to make an alignment of amino acid sequences with human MC4-R in all regions, modeling may be performed on the seven helix sites alone that can be determined using hydrophobicity or the like as an indicator. Whether a given set of atomic coordinates constituting a mutated GPCR amino acid sequence is included as a derivative of the present invention can be analyzed by comparing the three-dimensional structure of the GPCR in question and native human MC4-R (as represented by Table 1) by using as a parameter the discrepancies between the positions of α carbon atoms of specific amino acid residues.

Specifically, an image of the three-dimensional structure composed of the atomic coordinates of native human MC4-R (designated “three-dimensional structure I”) and an image of the three-dimensional structure composed of the derived coordinates in question (designated “three-dimensional structure II”) are created on computer based on the two sets of atomic coordinates. Subsequently, one image is superimposed on the other image on a computer display. The image of three-dimensional structure I contains seven helix sites of human MC4-R. These regions are superimposed on the regions of the corresponding helix sites in the image of three-dimensional structure II, or vice versa. This superimposition may be processed automatically based on a computer program, or may be carried out manually on a computer display in a manner similar to the one employed in comparing fingerprints. A suitable program includes COMPOSER or MODELER.

The seven helix sites (regions) and three extra-cellular sites (regions) of human MC4-R include the first helix (expressed as “HI(41-70)”) consisting of an amino acid sequence from position 41 to position 70 (SEQ ID NO: 2) of the amino acid sequence as shown in SEQ ID NO: 1 (hereinafter, all the position numbers mentioned in this paragraph refer to position numbers in SEQ ID NO: 1), the second helix (expressed as “HII (78-107)”) consisting of an amino acid sequence from position 78 to position 107 (SEQ ID NO: 3), the third helix (expressed as “HIII (120-150)”) consisting of an amino acid sequence from position 120 to position 150 (SEQ ID NO: 4), the fourth helix (expressed as “HIV (163-186)”) consisting of an amino acid sequence from position 163 to position 186 (SEQ ID NO: 5), the fifth helix (expressed as “HV (189-217)”) consisting of an amino acid sequence from position 189 to position 217 (SEQ ID NO: 6), the sixth helix (expressed as “H-VI(239-270)”) consisting of an amino acid sequence from position 239 to position 270 (SEQ ID NO: 7), the seventh helix (expressed as “HVII(282-305)”) consisting of an amino acid sequence from position 282 to position 305 (SEQ ID NO: 8); the first extra-cellular region (expressed as “ECI (108-119)”) consisting of an amino acid sequence from position 107 to position 119 (SEQ ID NO: 9), the second extra-cellular region (expressed as “ECII (187-188)”) consisting of an amino acid sequence from position 187 to position 188 (SEQ ID NO: 10), and the third extra-cellular region (expressed as “ECIII (271-281)”) consisting of an amino acid sequence from position 271 to position 281 (SEQ ID NO: 11). Then, these regions are superimposed on the corresponding regions of the three-dimensional structure II or vice versa, to calculate the discrepancies between the positions of the α carbon atoms in the amino acid residues of those helices in the three-dimensional structure I and the positions of the corresponding α carbon atoms in the amino acid residues of the corresponding helices in the three-dimensional structure II. When the mean residual of the discrepancies is 1.5 Å, preferably 1.0, more preferably 0.5, or less, the three-dimensional structure II is regarded as a derivative of the atomic coordinates as shown in Table 1 (derived coordinates). Discrepancies between the positions of corresponding α carbon atoms can be calculated by the so-called least squares method. Generally, such discrepancies can be calculated using a computer software (e.g., LSQKAB, Quanta). The mean residual is calculated as follows. Briefly, individual distances between two corresponding α carbon atoms are squared; the mean value of these squares is calculated; and then the square root of the mean value is calculated to obtain the mean residual.

An active site or protein-binding site is formed by at least one helix selected from the above-described seven helices. Accordingly, a G protein-coupled receptor that has a three-dimensional structure of that active site or protein-binding site, preferably at least one helix consists of HIII, HV, HVI and HVII, is also included in the G protein-coupled receptor of the invention.

In addition to the GPCR (human MC4-R) having the three-dimensional structure consisting of the atomic coordinates as shown in Table 1, the present invention also includes a three-dimensional structure consisting of such derived coordinates as the GPCRs of the invention.

In Table 1, atomic coordinates for the amino acid residues of individual helices and extra-cellular regions corresponds to amino acid residues 41-70 for HI, 78-107 for HII, 108-119 for ECI, 120-150 for HIII, 163-186 for HIV, 187-188 for ECII, 189-217 for HV, 239-270 for HVI, 271-281 for ECIII and 282-305 for HVII (residue numbers are entered in the 5th column).

II. Uses of the 3D Coordinates of MC4-R

For the first time, the present invention permits the use of virtual design techniques (i.e., computer modeling or “in silico”) to design, select, and synthesize a drug candidate capable of inhibiting/stimulating or binding MC4-R. In turn, these drug candidates may be effective in the treatment of a MC4-R mediated disorder.

The term “MC4-R mediated disorder” is disorder that involves a modulation in MC4-R activity in the biological manifestation of the disease, disorder, and/or condition; in the biological cascade leading to the disorder; or as a symptom of the disorder. This “involvement” of MC4-R in a MC4-R mediated disorder includes, but is not limited to, the following: (1) The modulation of MC4-R activity as a “cause” of the disorder or biological manifestation, whether the MC4-R is modulated genetically, by infection, by autoimmunity, trauma, biomechanical causes, lifestyle, or by some other causes. (2) The modulated MC4-R activity is part of the observable manifestation of the disease or disorder. That is, the disease or disorder is measurable in terms of the modulated MC4-R activity. From a clinical standpoint, modulated MC4-R activity indicates the disease, however, MC4-R activity need not be the “hallmark” of the disease or disorder. (3) The modulated MC4-R activity is part of the biochemical or cellular cascade that results in the disease or disorder. In this respect, inhibiting or stimulating of MC4-R (per the respective therapeutic goal) interrupts the cascade, and thus controls the disease. “Modulation in MC4-R activity,” as used herein, encompasses both unwanted or elevated MC4-R activity and desired or reduced MC4-R activity.

Non-limiting examples of MC4-R mediated disorders that may be treated by agonists or antagonists identified by the present invention include obesity, anorexia, inflammation, mental disorders, cachexia, metabolic and eating disorders, diabetes, insulin resistance, glucose intolerance, Type-2 diabetes mellitus, coronary artery disease, elevated blood pressure, hypertension, dyslipidaeniia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease, memory (including learning), cardiovascular function, sepsis, cardiogenic and hypovolemic shock, sexual dysfunction, penile erection, muscle atrophy, nerve growth and repair, intrauterine fetal growth, and the like.

In the present invention, it is possible to carry out virtual screening for drugs using the above-described atomic coordinates or derived coordinates therefrom.

Briefly, the atomic coordinates of the three-dimensional structure elucidated by the invention are input into a computer so that images of the structure and various parameters are shown on the display. All the information contained in Table 1 may be input, or atomic coordinates corresponding to at least one helix, or atomic coordinates corresponding to at least one extra-cellular region, or atomic coordinates corresponding to two helices linked by an extra-cellular region, or atomic coordinates corresponding to the top fifteen residues of at least one helix, or atomic coordinates corresponding to the top fifteen residues of two helices linked by an extra-cellular region may be input. For example, atomic coordinates corresponding to HI (41-70), HII (78-107), HIII (120-150), HIV (163-186), HV (189-217), HVI (239-270), HVII (282-305), ECI (108-119), ECII (187-188), ECIII (271-281), THII-ECI-THIII (93-134), THIV-ECII-THV (172-203), THVI-ECIII-THVII (256-296), THI (41-55), THII (93-107), THIII (120-134), THIV (172-186), THV (189-203), THVI (256-270), and/or THVII (282-296) may be input.

Then, the resultant data are input into a virtual compound library. Since a virtual compound library is contained in a virtual screening software such as DOCK-4 (Kuntz, UCSF), the above-described data may be input into such a software. Candidate drugs may be searched for using a three-dimensional structure database of virtual or non-virtual drug candidate compounds such as MDDR (Prous Science, Spain).

The potential stimulating/inhibitory or binding effect (i.e., interaction or association) of a drug candidate compound may be analyzed prior to its actual synthesis and testing by the use of computer modeling techniques. If the theoretical structure of the given compound suggests insufficient interaction and association between it and MC4-R, synthesis and testing of the candidate is obviated. However, if computer modeling indicates a strong interaction, the molecule may then be synthesized and tested for its ability to bind to or stimulate/inhibit MC4-R using various methods known in the art. Examples of such methods include the measurement of second messenger responses, in particular cAMP, such as the use of modified cell systems yielding color reaction upon accumulation of second messenger elements such as cAMP, e.g. as described by Chen et al. 1995 (Anal Biochem. 1995, 226, 349-54), Cytosensor Microphysiometer techniques (see Boyfield et al. 1996). In this manner, synthesis of inoperative candidates may be avoided.

Agonist/antagonist or binding drug candidates of MC4-R may be computationally evaluated and designed by means of a series of steps in which chemical entities or fragments are screened and selected for their ability to bind with the individual binding sites (e.g., HI, HV, HVI, HVII, ECI, ECII, ECIII) or other areas of MC4-R.

One skilled in the art may use one of several methods to screen chemical entities or fragments for their ability to associate with MC4-R and more particularly with the individual binding sites. This process may begin by visual inspection of, for example, the active site on the computer screen based on the MC4-R coordinates in Table I. Selected fragments or chemical entities may then be positioned in a variety of orientations, or docked, within an individual binding site of MC4-R as defined supra. Docking may be accomplished using software such as QUANTA, SYBYL, followed by energy minimization and molecular dynamics with standard molecular mechanics forcefields, such as CHARMM and AMBER.

Specialized computer programs may also assist in the process of selecting fragments or chemical entities. These include: (1) GRID (Goodford, P. J., “A Computational Procedure for Determining Energetically Favorable Binding Sites on Biologically Important Macromolecules” J. Med. Chem., 28, pp. 849-857 (1985)), available from Oxford University, Oxford, UK; (2) MCSS (Miranker, A. and M. Karplus, “Functionality Maps of Binding Sites: A Multiple Copy Simultaneous Search Method.” Proteins: Structure, Function and Genetics, 11, pp. 29-34 (1991)), available from Molecular Simulations, Burlington, Mass; (3) AUTODOCK (Goodsell, D. S. and A. J. Olsen, “Automated Docking of Substrates to Proteins by Simulated Annealing” Proteins: Structure. Function, and Genetics, 8, pp. 195-202 (1990)), available from Scripps Research Institute, La Jolla, Calif.; and (4) DOCK (Kuntz, I. D. et al., “A Geometric Approach to Macromolecule-Ligand Interactions” J. Mol. Biol., 161, pp. 269-288 (1982)), available from University of California, San Francisco, Calif; (5) GLIDE available from Schrodinger Inc.; (6) FlexX available from Tripos Inc; (7) GOLD (Jones et al., J. Mol. Biol., 245, 43-53, 1995) available from the Cambridge Crystallographic Data Centre.

Once suitable chemical entities or fragments have been selected, they can be assembled in silico or synthesized into a single drug candidate. In silico assembly may proceed by visual inspection of the relationship of the fragments to each other on the three-dimensional image displayed on a computer screen in relation to the structure coordinates of MC4-R. This would be followed by manual model building using software such as QUANTA or SYBYL. Chemical syntheses are by those methods well-known in the art.

Useful programs to aid one of skill in the art in connecting the individual chemical entities or fragments include the following: (1) CAVEAT (Bartlett, P. A. et al, “CAVEAT: A Program to Facilitate the Structure-Derived Design of Biologically Active Molecules”. In Molecular Recognition in Chemical and Biological Problems”, Special Pub., Royal Chem. Soc., 78, pp. 182-196 (1989)), available from the University of California, Berkeley, Calif.; (2) 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)); and (3) HOOK (available from Molecular Simulations, Burlington, Mass.).

Instead of proceeding to build an MC4-R agonist or antagonist in a step-wise fashion one fragment or chemical entity at a time as described above, drug candidates useful for the treatment of MC4-R mediated disorders may be designed as a whole or “de novo” using either an empty active site or optionally including some portion(s) of a known agonist or antagonist. These methods include the following: (1) LUDI (Bohm, H.-J., “The Computer Program LUDI: A New Method for the De Novo Design of Enzyme Inhibitors”, J. ComR. Aid. Molec. Design, 6, pp. 61-78 (1992)), available from Biosym Technologies, San Diego, Calif.; (2) LEGEND (Nishibata, Y. and A. Itai, Tetrahedron, 47, p. 8985 (1991)), available from Molecular Simulations, Burlington, Mass.; (3) LeapFrog (available from Tripos Associates, St. Louis, Mo.). Examples of known agonist or antagonists are described in WO 02/26774 A2.

Once a drug candidate has been designed or selected by the above methods, the efficiency with which that candidate may bind to MC4-R may be tested and optimized by computational evaluation. An effective MC4-R agonist must preferably demonstrate a relatively small difference in energy between its bound and free states (i.e., a small deformation energy of binding). Thus, the most efficient MC4-R agonist should preferably be designed with a deformation energy of binding of not greater than about 10 kcal/mole, preferably, not greater than 7 kcal/mole. MC4-R agonists may interact with the enzyme in more than one conformation that is similar in overall binding energy. In those cases, the deformation energy of binding is taken to be the difference between the energy of the free compound and the average energy of the conformations observed when the agonist binds to the enzyme.

A compound designed or selected as binding to MC4-R may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the compound and the enzyme when the compound is bound to MC4-R, preferably make a neutral or favorable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. ©1992]; AMBER, version 4.0 [P. A. Kollman, University of California at San Francisco, ©1994]; QUANTA/CHARMM [Molecular Simulations, Inc., Burlington, Mass. ©1994]; and Insight II/Discover (Biosysm Technologies Inc., San Diego, Calif. ©1994). Other software packages will be known to those skilled in the art.

Once a MC4-R-binding compound has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to MC4-R by the same computer methods described in detail, above.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope.

1. A method of identifying a drug candidate useful for the treatment of a melanocortin receptors-4 (MC4-R) mediated disorder comprising the steps of:
(a) using a three-dimensional (3D) structure of MC4-R according to Table 1;
(b) employing said 3D structure to design or select in silico said drug candidate.

2. The method according to claim 1, further comprising the step of synthesizing said drug candidate.

3. The method according to claim 2, further comprising contacting said drug candidate in the presence of a substrate to test the ability of said candidate to modulate MC4-R.

4. The method according to claim 3, wherein said candidate is selected from a database of virtual or non-virtual compounds.

5. The method according to claim 3, wherein said candidate is designed de novo.

6. The method according to claim 3, wherein said candidate is designed from a known agonist or antagonist.

7. The method according to claim 1, wherein said step of employing said 3D structure to design or select said candidate comprises the steps of:
(a) identifying in silico chemical entities or fragments capable of binding with a peptide selected from the group consisting of SEQ ID NOS: 2-11; and
(b) assembling in silico or synthesizing the identified chemical entities or fragments in into a single molecule to provide the structure of said candidate.

8. The method according to claim 1, wherein said step of employing said 3D structure to design or select said candidate comprises the steps of:
(a) identifying in silico chemical entities or fragments capable of binding with a peptide selected from the group consisting of SEQ ID NOS: 12-15; and
(b) assembling in silico or synthesizing the identified chemical entities or fragments into a single molecule to provide the structure of said candidate.

9. The method according to claim 1, wherein said step of employing said 3D structure to design or select said candidate comprises the steps of:
(a) identifying in silico chemical entities or fragments capable of binding with a peptide selected from the group consisting of SEQ ID NOS: 12, 16-21; and
(b) assembling in silico or synthesizing the identified chemical entities or fragments into a single molecule to provide the structure of said candidate.

10. The method according to claim 1, wherein the candidate is an agonist or an antagonist of MC4-R.

11. A G-protein-coupled receptor selected from the group consisting of the following (a) or (b):
(a) a G protein-coupled receptor having three-dimensional structure I defined by the atomic coordinates as shown in Table 1;
(b) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.

12. The receptor of claim 11, wherein the G protein-coupled receptor is MC4-R.

13. The receptor of claim 11, wherein the G protein-coupled receptor is human MC4-R.

14. A method of identifying a drug candidate useful for the treatment of a melanocortin receptors-4 (MC4-R) mediated disorder comprising the steps of:
(a) using a three-dimensional (3D) structure of a G-protein-coupled receptor selected from the group consisting of the following (i) or (ii):
(i) a G protein-coupled receptor having three-dimensional structure I defined by the atomic coordinates as shown in Table 1;
(ii) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.
(b) employing said 3D structure to design or select said drug candidate.

15. The method according to claim 14, further comprising the step of synthesizing said drug candidate.

16. The method according to claim 15, further comprising contacting said drug candidate in the presence of a substrate to test the ability of said candidate to modulate MC4-R.

17. The method according to claim 16, wherein said candidate is selected from a database of virtual or non-virtual compounds.

18. The method according to claim 16, wherein said candidate is designed de novo.

19. The method according to claim 16, wherein said candidate is designed from a known agonist or antagonist.
